(19) United States
(12) Patent Application Publication
(10) Pub. No.: US 2026/0191825 A1
(43) Pub. Date: Jul. 9, 2026

(54) CONTRACEPTIVE COMPOUNDS AND DOSING FOR THE SAME

(71) Applicant: YourChoice Therapeutics, Inc., San Francisco, CA (US)

(72) Inventors: Akash Bakshi, San Francisco, CA (US); Nadja Mannowetz, San Francisco, CA (US)

(21) Appl. No.: 19/224,478

(22) Filed: May 30, 2025

Related U.S. Application Data
(60) Provisional application No. 63/653,669, filed on May 30, 2024.
(60) Provisional application No. 63/654,812, filed on May 31, 2024.
(60) Provisional application No. 63/784,878, filed on Apr. 7, 2025.

(51) Int. Cl. A61K 31/4025; A61P 15/16

(52) U.S. Cl. CPC A61K 31/4025; A61P 15/16

(57) ABSTRACT

The present disclosure describes methods of reducing the likelihood of conception following intercourse between a male subject and a female subject, methods of producing reversible infertility in a male subject, methods of reducing sperm count in a male subject, and methods of selectively antagonizing RAR alpha over RAR beta and RAR gamma in a subject.

### CROSS-REFERENCE TO RELATED APPLICATIONS

This application claims the benefit of U.S. Provisional Application No. 63/653,669, filed on May 30, 2024, U.S. Provisional Application No. 63/654,812, filed on May 31, 2024, and U.S. Provisional Application No. 63/784,878, filed on Apr. 7, 2025, which are incorporated herein by reference in their entirety.

### BACKGROUND

It is reported that approximately half of all pregnancies globally are unintended. Whereas women have multiple contraceptive options, limited male contraceptives are available, and options such as condoms or vasectomy are each associated with their own challenges. The failure rate of condoms is approximately 12% to 17%. Despite the reliable efficacy of vasectomies, a surgical procedure may be undesirable to men and reversibility is not always achieved. Therefore, there is a need to develop a safe, effective, reversible, and convenient male contraceptive option.

Retinoic acid signaling is a well-documented target for the regulation of spermatogenesis. Vitamin A deficiency is known to result in male sterility by the inhibition of spermatogenesis. Gene targeting research has also demonstrated spermatogenesis inhibition through the loss of function of retinoic acid synthetic enzymes, enzyme degradation, and effects on retinoic acid receptors (RARs), especially RAR-alpha. However, these genetic approaches to spermatogenesis inhibition are typically permanent. Pharmacological approaches have targeted the inhibition of spermatogenesis through impairing retinoic acid signaling in the testes, such as RAR antagonism. This approach has lead to the development of pan-antagonists, but a RAR-selective antagonist may lead to greater specificity at lower doses. RAR-alpha-selective antagonists are therefore promising candidates for inducing reversible infertility in males. It is contemplated that orally administering the compound of the present disclosure, a RAR-alpha antagonist, impairs spermatogenesis and spermiation in the testis and reduces both sperm count and sperm motility in animal studies.

### SUMMARY

In some aspects, the techniques described herein relate to a method of reducing the likelihood of conception following intercourse between a male subject and a female subject, including administering to the male subject prior to the intercourse a therapeutically effective amount of a compound of formula (I):

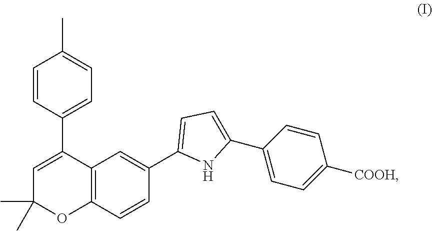 [SMILES: Cc1ccc(C2=CC(C)(C)Oc3ccc(-c4ccc(-c5ccc(C(=O)O)cc5)[nH]4)cc32)cc1]

or a pharmaceutically acceptable salt thereof; wherein the therapeutically effective amount is about 15 mg to about 450 mg.

In some aspects, the techniques described herein relate to a method, wherein the therapeutically effective amount is about 15 mg to about 180 mg.

In some aspects, the techniques described herein relate to a method, wherein the therapeutically effective amount is about 15 mg.

In some aspects, the techniques described herein relate to a method, wherein the therapeutically effective amount is about 30 mg.

In some aspects, the techniques described herein relate to a method, wherein the therapeutically effective amount is about 45 mg.

In some aspects, the techniques described herein relate to a method, wherein the therapeutically effective amount is about 60 mg.

In some aspects, the techniques described herein relate to a method, wherein the therapeutically effective amount is about 90 mg.

In some aspects, the techniques described herein relate to a method, wherein the therapeutically effective amount is about 180 mg.

In some aspects, the techniques described herein relate to a method, wherein the therapeutically effective amount is administered once per day for about 28 days to about 24 weeks.

In some aspects, the techniques described herein relate to a method, wherein the therapeutically effective amount is administered once per day for about 13 weeks to about 24 weeks.

In some aspects, the techniques described herein relate to a method, wherein the therapeutically effective amount is administered once per day for about 13 weeks.

In some aspects, the techniques described herein relate to a method, wherein the therapeutically effective amount is administered once per day for about 20 weeks.

In some aspects, the techniques described herein relate to a method, wherein the therapeutically effective amount is administered once per day for about 24 weeks.

In some aspects, the techniques described herein relate to a method of producing reversible infertility in a male subject, including administering to the male subject including administering to the male subject a therapeutically effective amount of a compound of formula (I):

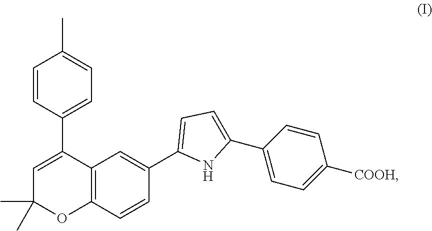 [SMILES: Cc1ccc(C2=CC(C)(C)Oc3ccc(-c4ccc(-c5ccc(C(=O)O)cc5)[nH]4)cc32)cc1]

or a pharmaceutically acceptable salt thereof; wherein the therapeutically effective amount is about 15 mg to about 450 mg.

In some aspects, the techniques described herein relate to a method, wherein the therapeutically effective amount is about 15 mg to about 180 mg.

In some aspects, the techniques described herein relate to a method, wherein the therapeutically effective amount is about 15 mg.

In some aspects, the techniques described herein relate to a method, wherein the therapeutically effective amount is about 30 mg.

In some aspects, the techniques described herein relate to a method, wherein the therapeutically effective amount is about 45 mg.

In some aspects, the techniques described herein relate to a method, wherein the therapeutically effective amount is about 60 mg.

In some aspects, the techniques described herein relate to a method, wherein the therapeutically effective amount is about 90 mg.

In some aspects, the techniques described herein relate to a method, wherein the therapeutically effective amount is about 180 mg.

In some aspects, the techniques described herein relate to a method, wherein the therapeutically effective amount is administered once per day for about 28 days to about 24 weeks.

In some aspects, the techniques described herein relate to a method, wherein the therapeutically effective amount is administered once per day for about 13 weeks to about 24 weeks.

In some aspects, the techniques described herein relate to a method, wherein the therapeutically effective amount is administered once per day for about 13 weeks.

In some aspects, the techniques described herein relate to a method, wherein the therapeutically effective amount is administered once per day for about 20 weeks.

In some aspects, the techniques described herein relate to a method, wherein the therapeutically effective amount is administered once per day for about 24 weeks.

In some aspects, the techniques described herein relate to a method of reducing sperm count in a male subject, including administering to the male subject a therapeutically effective amount of a compound of formula (I):

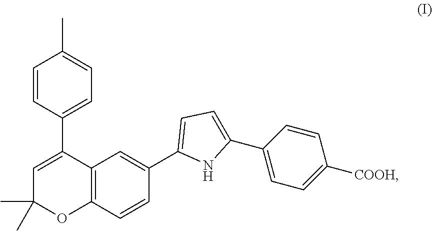 [SMILES: Cc1ccc(C2=CC(C)(C)Oc3ccc(-c4ccc(-c5ccc(C(=O)O)cc5)[nH]4)cc32)cc1]

or a pharmaceutically acceptable salt thereof; wherein the therapeutically effective amount is about 15 mg to about 450 mg.

In some aspects, the techniques described herein relate to a method, wherein the therapeutically effective amount is about 15 mg to about 180 mg.

In some aspects, the techniques described herein relate to a method, wherein the therapeutically effective amount is about 15 mg.

In some aspects, the techniques described herein relate to a method, wherein the therapeutically effective amount is about 30 mg.

In some aspects, the techniques described herein relate to a method, wherein the therapeutically effective amount is about 45 mg.

In some aspects, the techniques described herein relate to a method, wherein the therapeutically effective amount is about 60 mg.

In some aspects, the techniques described herein relate to a method, wherein the therapeutically effective amount is about 90 mg.

In some aspects, the techniques described herein relate to a method, wherein the therapeutically effective amount is about 180 mg.

In some aspects, the techniques described herein relate to a method, wherein the therapeutically effective amount is administered once per day for about 28 days to about 24 weeks.

In some aspects, the techniques described herein relate to a method, wherein the therapeutically effective amount is administered once per day for about 13 weeks to about 24 weeks.

In some aspects, the techniques described herein relate to a method, wherein the therapeutically effective amount is administered once per day for about 13 weeks.

In some aspects, the techniques described herein relate to a method, wherein the therapeutically effective amount is administered once per day for about 20 weeks.

In some aspects, the techniques described herein relate to a method, wherein the therapeutically effective amount is administered once per day for about 24 weeks.

In some aspects, the techniques described herein relate to a method of selectively antagonizing RAR alpha over RAR beta and RAR gamma in a subject, including administering to the subject a therapeutically effective amount of a compound of formula (I):

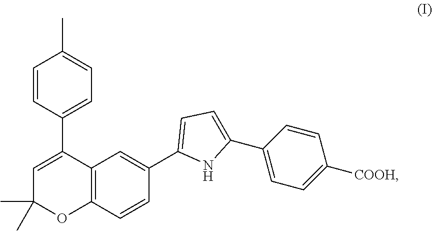 [SMILES: Cc1ccc(C2=CC(C)(C)Oc3ccc(-c4ccc(-c5ccc(C(=O)O)cc5)[nH]4)cc32)cc1]

or a pharmaceutically acceptable salt thereof; wherein the therapeutically effective amount is about 15 mg to about 450 mg.

In some aspects, the techniques described herein relate to a method, wherein the therapeutically effective amount is about 15 mg to about 180 mg.

In some aspects, the techniques described herein relate to a method, wherein the therapeutically effective amount is about 15 mg.

In some aspects, the techniques described herein relate to a method, wherein the therapeutically effective amount is about 30 mg.

In some aspects, the techniques described herein relate to a method, wherein the therapeutically effective amount is about 45 mg.

In some aspects, the techniques described herein relate to a method, wherein the therapeutically effective amount is about 60 mg.

In some aspects, the techniques described herein relate to a method, wherein the therapeutically effective amount is about 90 mg.

In some aspects, the techniques described herein relate to a method, wherein the therapeutically effective amount is about 180 mg.

In some aspects, the techniques described herein relate to a method, wherein the therapeutically effective amount is administered once per day for about 28 days to about 24 weeks.

In some aspects, the techniques described herein relate to a method, wherein the therapeutically effective amount is administered once per day for about 13 weeks to about 24 weeks.

In some aspects, the techniques described herein relate to a method, wherein the therapeutically effective amount is administered once per day for about 13 weeks.

In some aspects, the techniques described herein relate to a method, wherein the therapeutically effective amount is administered once per day for about 20 weeks.

In some aspects, the techniques described herein relate to a method, wherein the therapeutically effective amount is administered once per day for about 24 weeks.

### DRAWINGS

Aspects, features, benefits, and advantages of the embodiments described herein will be apparent with regard to the following description, appended claims, and accompanying drawings where:

FIG. 1A is a cross-section of a canine testicle prior to dosing with the compound of the present disclosure, showing developing and mature sperm. FIG. 1B is a cross-section of a canine testicle after dosing with the compound of the present disclosure, showing no developing and mature sperm. FIG. 1C is a cross-section of a canine testicle after a recovery period of no dosing with the compound of the present disclosure, showing the return of developing sperm.

FIG. 2A is a graph of sperm count in non-human primates (NHPs) after dosing with the compound of the present disclosure for 108 days followed by a recovery period. FIG. 2B is a graph of sperm count in non-human primates (NHPs) after dosing with the compound of the present disclosure for 37 days followed by a recovery period. FIG. 2C is a bar chart of sperm concentration pre-dosing, after two weeks of dosing, and at the end of recovery after dosing with the compound of the present disclosure.

FIG. 3A shows the disruption of spermatogenesis following the end of dosing with the compound of the present disclosure. FIG. 3B shows the germinal epithelium recovery following the end of the recovery period, according to an embodiment of the present disclosure.

FIG. 4A is a graph of concentration of the compound of the present disclosure following a single dose in humans over 24 hours, according to an embodiment of the present disclosure. FIG. 4B is a graph of concentration of the compound of the present disclosure following a single dose in humans over 14 days, according to an embodiment of the present disclosure.

FIGS. 5A-5C are graphs of concentration of the compound of the present disclosure following a single dose of 30 mg, 90 mg, and 180 mg, respectively, in six human patients over 14 days, according to an embodiment of the present disclosure.

FIGS. 6A and 6B are graphs of median simulated concentration of the compound of the present disclosure at various doses over 24 hours and 28 days, respectively, according to embodiments of the present disclosure.

FIG. 7A is a graph of concentration of the compound of the present disclosure in mice versus humans at various doses, according to an embodiment of the present disclosure.

FIG. 7B is a graph of concentration of the compound of the present disclosure in dogs versus humans at various doses, according to an embodiment of the present disclosure. FIG. 7C is a graph of concentration of the compound of the present disclosure in non-human primates at a dose of 2.5 mg/kg versus humans at various doses, according to an embodiment of the present disclosure. FIG. 7D is a graph of concentration of the compound of the present disclosure in non-human primates at a dose of 5 mg/kg versus humans at various doses, according to an embodiment of the present disclosure. HED=human equivalent dose.

FIG. 8 is a flow chart of the Phase 1a study design, according to an embodiment of the present disclosure.

FIG. 9 is a flow chart of the study design for Dose Escalation (Phase 1b Part) and Cohort Expansion (Phase 2a Part), according to an embodiment of the present disclosure. DERC=Dose Escalation Review Committee.

FIG. 10 is a flow chart of Study Sequence per Cohort in the Phase 1b Part, according to an embodiment of the present disclosure.

FIG. 11 is a flow chart of Study Sequence per Cohort in the Phase 2a Part, according to an embodiment of the present disclosure.

### DETAILED DESCRIPTION

Before compounds, compositions and methods are described in detail, it is to be understood that this disclosure is not limited to the particular processes, compositions, or methodologies described, as these may vary. It is also to be understood that the terminology used in the description is for the purpose of describing the particular versions or embodiments only, and is not intended to limit the scope of the disclosure which will be limited only by the appended claims. Unless defined otherwise, all technical and scientific terms used herein have the same meaning as commonly understood by one of ordinary skill in the art. Although any methods and materials similar or equivalent to those described herein can be used in the practice or testing of embodiments of the disclosure, the preferred methods, devices, and materials are now described.

It is further appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

As used herein, the term “about” means plus or minus up to 10% of the numerical value of the number with which it is being used. For example, “about 50%” means in the range of 45-55% and includes exactly 50%. The term “about” may refer to plus or minus 1%, 5%, or 10% of the numerical value of the number with which it is being used.

“Administering,” when used in conjunction with the compounds of the disclosure, means to administer a compound directly into or onto a target tissue or to administer a compound systemically or locally to a patient or other subject.

As used herein, the term “free base” refers to a non-salt form of a compound as described herein.

The term “salts” refers to the common meaning in the art. The salts as described herein may be pharmaceutically acceptable.

Those skilled in the art of organic chemistry will appreciate that many organic compounds can form complexes with solvents in which they are reacted or from which they are precipitated or crystallized. For example, a complex with water is known as a “hydrate.” Solvates of the compounds of the present disclosure are within the scope of this disclosure. The salts of the compound of any of the formulae described herein may form solvates (e.g., hydrates) and the present disclosure includes all such solvates. The meaning of the word “solvates” is well known to those skilled in the art as a compound formed by interaction of a solvent and a solute (i.e., solvation). Techniques for the preparation of solvates are well established in the art.

The phrase “pharmaceutically acceptable” refers to molecular entities and compositions that are generally regarded as safe and nontoxic. In particular, pharmaceutically acceptable carriers, diluents or other excipients used in the pharmaceutical compositions of this disclosure are physiologically tolerable, compatible with other ingredients, and do not typically produce an allergic or similar untoward reaction (for example, gastric upset, dizziness and the like) when administered to a subject. In some embodiments, as used herein, the term “pharmaceutically acceptable” means approved by a regulatory agency of the Federal or a state government or listed in the U.S. Pharmacopoeia or other generally recognized pharmacopoeia for use in animals, and more particularly in humans. The phrase “pharmaceutically acceptable salt(s)”, as used herein, includes those salts of compounds of the disclosure that are safe and effective for use in mammals and that possess the desired biological activity. Pharmaceutically acceptable salts include salts of acidic or basic groups present in compounds of the disclosure or in compounds identified pursuant to the methods of the disclosure.

As used herein, the term “therapeutic” means an agent utilized to treat, combat, ameliorate, protect against, or improve an unwanted condition or disease of a subject.

A “therapeutically effective amount” or “effective amount” of a compound, such as vapendavir, or composition of the disclosure is a predetermined amount which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect or physician observes a change). The effect contemplated herein includes medical therapeutic treatment, as appropriate. The specific dose of a compound administered according to this disclosure to obtain therapeutic effects will, of course, be determined by the particular circumstances surrounding the case, including, for example, the compound administered, the route of administration, the co-administration of other active ingredients, the condition being treated, the activity of the specific compound employed, the specific composition employed, the age, body weight, general health, sex and diet of the subject; the time of administration, route of administration, and rate of excretion of the specific compound employed and the duration of the treatment. The effective amount administered will be determined by the physician in the light of the foregoing relevant circumstances and the exercise of sound medical judgment. A therapeutically effective amount of a compound of this disclosure, such as vapendavir, is typically an amount such that when it is administered in a physiologically tolerable excipient composition, it is sufficient to achieve an effective systemic concentration or local concentration in the tissue.

The terms “treat”, “treated”, or “treating” as used herein refers to therapeutic treatment measures, wherein the object is to protect against (partially or wholly) or slow down (e.g., lessen or postpone the onset of) an undesired physiological condition, disorder or disease, or to obtain beneficial or desired clinical results such as partial or total restoration or inhibition in decline of a parameter, value, function or result that had or would become abnormal. For the purposes of this disclosure, beneficial or desired clinical results include, but are not limited to, alleviation of symptoms; diminishment of the extent or vigor or rate of development of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether or not it translates to immediate lessening of actual clinical symptoms, or enhancement or improvement of the condition, disorder or disease. Treatment seeks to elicit a clinically significant response without excessive levels of side effects. Treatment also includes prolonging survival as compared to expected survival if not receiving treatment.

The compound of the present disclosure, a compound of formula (I):

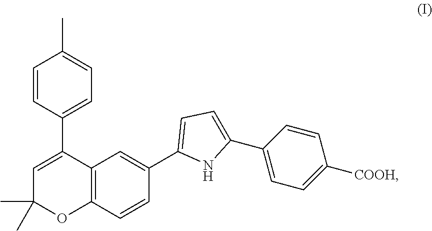 [SMILES: Cc1ccc(C2=CC(C)(C)Oc3ccc(-c4ccc(-c5ccc(C(=O)O)cc5)[nH]4)cc32)cc1]

which may also be referred to herein as Compound A, is a highly selective RAR-alpha antagonist that reversibly reduces sperm counts and induces male infertility.

In embodiments, the present disclosure describes a method of reducing the likelihood of conception following intercourse between a male subject and a female subject, the method including administering to the male subject prior to the intercourse a therapeutically effective amount of a compound of formula (I):

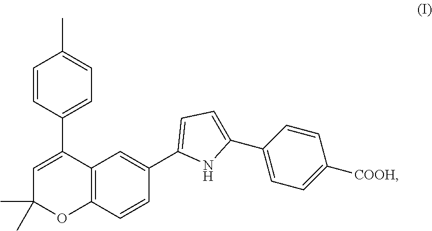 [SMILES: Cc1ccc(C2=CC(C)(C)Oc3ccc(-c4ccc(-c5ccc(C(=O)O)cc5)[nH]4)cc32)cc1]

or a pharmaceutically acceptable salt thereof, wherein the therapeutically effective amount is about 15 mg to about 450 mg.

In embodiments, the therapeutically effective amount to reduce the likelihood of conception following intercourse between a male subject and a female subject may be about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, or any value contained within a range formed by any two of the preceding values.

For example, in embodiments, the therapeutically effective amount may be about 15 mg to about 180 mg, about 15 mg to about 150 mg, about 15 mg to about 100 mg, about 15 mg to about 80 mg, about 15 mg to about 50 mg, or any value contained within a range formed by any two of the preceding values. In embodiments, the therapeutically effective amount may be about 180 mg to about 450 mg, about 200 mg to about 450 mg, about 250 mg to about 450 mg, about 300 mg to about 450 mg, about 350 mg to about 450 mg, or any value contained within a range formed by any two of the preceding values.

In embodiments, the therapeutically effective amount to reduce the likelihood of conception following intercourse between a male subject and a female subject may be about 15 mg. In embodiments, the therapeutically effective amount to reduce the likelihood of conception following intercourse between a male subject and a female subject may be about 20 mg. In embodiments, the therapeutically effective amount to reduce the likelihood of conception following intercourse between a male subject and a female subject may be about 25 mg. In embodiments, the therapeutically effective amount to reduce the likelihood of conception following intercourse between a male subject and a female subject may be about 30 mg. In embodiments, the therapeutically effective amount to reduce the likelihood of conception following intercourse between a male subject and a female subject may be about 35 mg. In embodiments, the therapeutically effective amount to reduce the likelihood of conception following intercourse between a male subject and a female subject may be about 40 mg. In embodiments, the therapeutically effective amount to reduce the likelihood of conception following intercourse between a male subject and a female subject may be about 45 mg. In embodiments, the therapeutically effective amount to reduce the likelihood of conception following intercourse between a male subject and a female subject may be about 50 mg. In embodiments, the therapeutically effective amount to reduce the likelihood of conception following intercourse between a male subject and a female subject may be about 55 mg. In embodiments, the therapeutically effective amount to reduce the likelihood of conception following intercourse between a male subject and a female subject may be about 60 mg. In embodiments, the therapeutically effective amount to reduce the likelihood of conception following intercourse between a male subject and a female subject may be about 65 mg. In embodiments, the therapeutically effective amount to reduce the likelihood of conception following intercourse between a male subject and a female subject may be about 70 mg. In embodiments, the therapeutically effective amount to reduce the likelihood of conception following intercourse between a male subject and a female subject may be about 75 mg. In embodiments, the therapeutically effective amount to reduce the likelihood of conception following intercourse between a male subject and a female subject may be about 80 mg. In embodiments, the therapeutically effective amount to reduce the likelihood of conception following intercourse between a male subject and a female subject may be about 85 mg. In embodiments, the therapeutically effective amount to reduce the likelihood of conception following intercourse between a male subject and a female subject may be about 90 mg. In embodiments, the therapeutically effective amount to reduce the likelihood of conception following intercourse between a male subject and a female subject may be about 95 mg. In embodiments, the therapeutically effective amount to reduce the likelihood of conception following intercourse between a male subject and a female subject may be about 100 mg. In embodiments, the therapeutically effective amount to reduce the likelihood of conception following intercourse between a male subject and a female subject may be about 105 mg. In embodiments, the therapeutically effective amount to reduce the likelihood of conception following intercourse between a male subject and a female subject may be about 110 mg. In embodiments, the therapeutically effective amount to reduce the likelihood of conception following intercourse between a male subject and a female subject may be about 115 mg. In embodiments, the therapeutically effective amount to reduce the likelihood of conception following intercourse between a male subject and a female subject may be about 120 mg. In embodiments, the therapeutically effective amount to reduce the likelihood of conception following intercourse between a male subject and a female subject may be about 125 mg. In embodiments, the therapeutically effective amount to reduce the likelihood of conception following intercourse between a male subject and a female subject may be about 130 mg. In embodiments, the therapeutically effective amount to reduce the likelihood of conception following intercourse between a male subject and a female subject may be about 135 mg. In embodiments, the therapeutically effective amount to reduce the likelihood of conception following intercourse between a male subject and a female subject may be about 140 mg. In embodiments, the therapeutically effective amount to reduce the likelihood of conception following intercourse between a male subject and a female subject may be about 145 mg. In embodiments, the therapeutically effective amount to reduce the likelihood of conception following intercourse between a male subject and a female subject may be about 150 mg. In embodiments, the therapeutically effective amount to reduce the likelihood of conception following intercourse between a male subject and a female subject may be about 155 mg. In embodiments, the therapeutically effective amount to reduce the likelihood of conception following intercourse between a male subject and a female subject may be about 160 mg. In embodiments, the therapeutically effective amount to reduce the likelihood of conception following intercourse between a male subject and a female subject may be about 165 mg. In embodiments, the therapeutically effective amount to reduce the likelihood of conception following intercourse between a male subject and a female subject may be about 170 mg. In embodiments, the therapeutically effective amount to reduce the likelihood of conception following intercourse between a male subject and a female subject may be about 175 mg. In embodiments, the therapeutically effective amount to reduce the likelihood of conception following intercourse between a male subject and a female subject may be about 180 mg. In embodiments, the therapeutically effective amount to reduce the likelihood of conception following intercourse between a male subject and a female subject may be about 185 mg. In embodiments, the therapeutically effective amount to reduce the likelihood of conception following intercourse between a male subject and a female subject may be about 190 mg. In embodiments, the therapeutically effective amount to reduce the likelihood of conception following intercourse between a male subject and a female subject may be about 195 mg. In embodiments, the therapeutically effective amount to reduce the likelihood of conception following intercourse between a male subject and a female subject may be about 200 mg. In embodiments, the therapeutically effective amount to reduce the likelihood of conception following intercourse between a male subject and a female subject may be about 205 mg. In embodiments, the therapeutically effective amount to reduce the likelihood of conception following intercourse between a male subject and a female subject may be about 210 mg. In embodiments, the therapeutically effective amount to reduce the likelihood of conception following intercourse between a male subject and a female subject may be about 215 mg. In embodiments, the therapeutically effective amount to reduce the likelihood of conception following intercourse between a male subject and a female subject may be about 220 mg. In embodiments, the therapeutically effective amount to reduce the likelihood of conception following intercourse between a male subject and a female subject may be about 225 mg. In embodiments, the therapeutically effective amount to reduce the likelihood of conception following intercourse between a male subject and a female subject may be about 230 mg. In embodiments, the therapeutically effective amount to reduce the likelihood of conception following intercourse between a male subject and a female subject may be about 235 mg. In embodiments, the therapeutically effective amount to reduce the likelihood of conception following intercourse between a male subject and a female subject may be about 240 mg. In embodiments, the therapeutically effective amount to reduce the likelihood of conception following intercourse between a male subject and a female subject may be about 245 mg. In embodiments, the therapeutically effective amount to reduce the likelihood of conception following intercourse between a male subject and a female subject may be about 250 mg. In embodiments, the therapeutically effective amount to reduce the likelihood of conception following intercourse between a male subject and a female subject may be about 255 mg. In embodiments, the therapeutically effective amount to reduce the likelihood of conception following intercourse between a male subject and a female subject may be about 260 mg. In embodiments, the therapeutically effective amount to reduce the likelihood of conception following intercourse between a male subject and a female subject may be about 265 mg. In embodiments, the therapeutically effective amount to reduce the likelihood of conception following intercourse between a male subject and a female subject may be about 270 mg. In embodiments, the therapeutically effective amount to reduce the likelihood of conception following intercourse between a male subject and a female subject may be about 275 mg. In embodiments, the therapeutically effective amount to reduce the likelihood of conception following intercourse between a male subject and a female subject may be about 280 mg. In embodiments, the therapeutically effective amount to reduce the likelihood of conception following intercourse between a male subject and a female subject may be about 285 mg. In embodiments, the therapeutically effective amount to reduce the likelihood of conception following intercourse between a male subject and a female subject may be about 290 mg. In embodiments, the therapeutically effective amount to reduce the likelihood of conception following intercourse between a male subject and a female subject may be about 295 mg. In embodiments, the therapeutically effective amount to reduce the likelihood of conception following intercourse between a male subject and a female subject may be about 300 mg. In embodiments, the therapeutically effective amount to reduce the likelihood of conception following intercourse between a male subject and a female subject may be about 305 mg. In embodiments, the therapeutically effective amount to reduce the likelihood of conception following intercourse between a male subject and a female subject may be about 310 mg. In embodiments, the therapeutically effective amount to reduce the likelihood of conception following intercourse between a male subject and a female subject may be about 315 mg. In embodiments, the therapeutically effective amount to reduce the likelihood of conception following intercourse between a male subject and a female subject may be about 320 mg. In embodiments, the therapeutically effective amount to reduce the likelihood of conception following intercourse between a male subject and a female subject may be about 325 mg. In embodiments, the therapeutically effective amount to reduce the likelihood of conception following intercourse between a male subject and a female subject may be about 330 mg. In embodiments, the therapeutically effective amount to reduce the likelihood of conception following intercourse between a male subject and a female subject may be about 335 mg. In embodiments, the therapeutically effective amount to reduce the likelihood of conception following intercourse between a male subject and a female subject may be about 340 mg. In embodiments, the therapeutically effective amount to reduce the likelihood of conception following intercourse between a male subject and a female subject may be about 345 mg. In embodiments, the therapeutically effective amount to reduce the likelihood of conception following intercourse between a male subject and a female subject may be about 350 mg. In embodiments, the therapeutically effective amount to reduce the likelihood of conception following intercourse between a male subject and a female subject may be about 355 mg. In embodiments, the therapeutically effective amount to reduce the likelihood of conception following intercourse between a male subject and a female subject may be about 360 mg. In embodiments, the therapeutically effective amount to reduce the likelihood of conception following intercourse between a male subject and a female subject may be about 365 mg. In embodiments, the therapeutically effective amount to reduce the likelihood of conception following intercourse between a male subject and a female subject may be about 370 mg. In embodiments, the therapeutically effective amount to reduce the likelihood of conception following intercourse between a male subject and a female subject may be about 375 mg. In embodiments, the therapeutically effective amount to reduce the likelihood of conception following intercourse between a male subject and a female subject may be about 380 mg. In embodiments, the therapeutically effective amount to reduce the likelihood of conception following intercourse between a male subject and a female subject may be about 385 mg. In embodiments, the therapeutically effective amount to reduce the likelihood of conception following intercourse between a male subject and a female subject may be about 390 mg. In embodiments, the therapeutically effective amount to reduce the likelihood of conception following intercourse between a male subject and a female subject may be about 395 mg. In embodiments, the therapeutically effective amount to reduce the likelihood of conception following intercourse between a male subject and a female subject may be about 400 mg. In embodiments, the therapeutically effective amount to reduce the likelihood of conception following intercourse between a male subject and a female subject may be about 405 mg. In embodiments, the therapeutically effective amount to reduce the likelihood of conception following intercourse between a male subject and a female subject may be about 410 mg. In embodiments, the therapeutically effective amount to reduce the likelihood of conception following intercourse between a male subject and a female subject may be about 415 mg. In embodiments, the therapeutically effective amount to reduce the likelihood of conception following intercourse between a male subject and a female subject may be about 420 mg. In embodiments, the therapeutically effective amount to reduce the likelihood of conception following intercourse between a male subject and a female subject may be about 425 mg. In embodiments, the therapeutically effective amount to reduce the likelihood of conception following intercourse between a male subject and a female subject may be about 430 mg. In embodiments, the therapeutically effective amount to reduce the likelihood of conception following intercourse between a male subject and a female subject may be about 435 mg. In embodiments, the therapeutically effective amount to reduce the likelihood of conception following intercourse between a male subject and a female subject may be about 440 mg. In embodiments, the therapeutically effective amount to reduce the likelihood of conception following intercourse between a male subject and a female subject may be about 445 mg. In embodiments, the therapeutically effective amount to reduce the likelihood of conception following intercourse between a male subject and a female subject may be about 450 mg.

In embodiments, the therapeutically effective amount is administered once per day for about 28 days to about 24 weeks. In embodiments, the therapeutically effective amount is administered once per day for about 28 days to about 24 weeks, such as about 28 days, about 29 days, about 30 days, about 31 days, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, or any value contained within a range formed by any two of the preceding values.

For example, in embodiments, the therapeutically effective amount is administered once per day for about 13 weeks to about 24 weeks, about 13 weeks to about 20 weeks, about 16 weeks to about 24 weeks, about 20 weeks to about 24 weeks, or any value contained within a range formed by any two of the preceding values. In embodiments, the therapeutically effective amount is administered once per day for about 13 weeks. In embodiments, the therapeutically effective amount is administered once per day for about 14 weeks. In embodiments, the therapeutically effective amount is administered once per day for about 15 weeks. In embodiments, the therapeutically effective amount is administered once per day for about 16 weeks. In embodiments, the therapeutically effective amount is administered once per day for about 17 weeks. In embodiments, the therapeutically effective amount is administered once per day for about 18 weeks. In embodiments, the therapeutically effective amount is administered once per day for about 19 weeks. In embodiments, the therapeutically effective amount is administered once per day for about 20 weeks. In embodiments, the therapeutically effective amount is administered once per day for about 21 weeks. In embodiments, the therapeutically effective amount is administered once per day for about 22 weeks. In embodiments, the therapeutically effective amount is administered once per day for about 23 weeks. In embodiments, the therapeutically effective amount is administered once per day for about 24 weeks.

In embodiments, the therapeutically effective amount is about 15 mg and the therapeutically effective amount is administered once per day for about 13 weeks. In embodiments, the therapeutically effective amount is about 30 mg and the therapeutically effective amount is administered once per day for about 13 weeks. In embodiments, the therapeutically effective amount is about 45 mg and the therapeutically effective amount is administered once per day for about 13 weeks. In embodiments, the therapeutically effective amount is about 90 mg and the therapeutically effective amount is administered once per day for about 13 weeks. In embodiments, the therapeutically effective amount is about 180 mg and the therapeutically effective amount is administered once per day for about 13 weeks. In embodiments, the therapeutically effective amount is about 270 mg and the therapeutically effective amount is administered once per day for about 13 weeks. In embodiments, the therapeutically effective amount is about 300 mg and the therapeutically effective amount is administered once per day for about 13 weeks. In embodiments, the therapeutically effective amount is about 360 mg and the therapeutically effective amount is administered once per day for about 13 weeks. In embodiments, the therapeutically effective amount is about 450 mg and the therapeutically effective amount is administered once per day for about 13 weeks.

In embodiments, the therapeutically effective amount is about 15 mg and the therapeutically effective amount is administered once per day for about 20 weeks. In embodiments, the therapeutically effective amount is about 30 mg and the therapeutically effective amount is administered once per day for about 20 weeks. In embodiments, the therapeutically effective amount is about 45 mg and the therapeutically effective amount is administered once per day for about 20 weeks. In embodiments, the therapeutically effective amount is about 90 mg and the therapeutically effective amount is administered once per day for about 20 weeks. In embodiments, the therapeutically effective amount is about 180 mg and the therapeutically effective amount is administered once per day for about 20 weeks. In embodiments, the therapeutically effective amount is about 270 mg and the therapeutically effective amount is administered once per day for about 20 weeks. In embodiments, the therapeutically effective amount is about 300 mg and the therapeutically effective amount is administered once per day for about 20 weeks. In embodiments, the therapeutically effective amount is about 360 mg and the therapeutically effective amount is administered once per day for about 20 weeks. In embodiments, the therapeutically effective amount is about 450 mg and the therapeutically effective amount is administered once per day for about 20 weeks.

In embodiments, the therapeutically effective amount is about 15 mg and the therapeutically effective amount is administered once per day for about 24 weeks. In embodiments, the therapeutically effective amount is about 30 mg and the therapeutically effective amount is administered once per day for about 24 weeks. In embodiments, the therapeutically effective amount is about 45 mg and the therapeutically effective amount is administered once per day for about 24 weeks. In embodiments, the therapeutically effective amount is about 90 mg and the therapeutically effective amount is administered once per day for about 24 weeks. In embodiments, the therapeutically effective amount is about 180 mg and the therapeutically effective amount is administered once per day for about 24 weeks. In embodiments, the therapeutically effective amount is about 270 mg and the therapeutically effective amount is administered once per day for about 24 weeks. In embodiments, the therapeutically effective amount is about 300 mg and the therapeutically effective amount is administered once per day for about 24 weeks. In embodiments, the therapeutically effective amount is about 360 mg and the therapeutically effective amount is administered once per day for about 24 weeks. In embodiments, the therapeutically effective amount is about 450 mg and the therapeutically effective amount is administered once per day for about 24 weeks.

In embodiments, the therapeutically effective amount may be administered once per day, twice per day, or three times per day. In embodiments, the therapeutically effective amount may be administered twice per day for about 28 days to about 24 weeks. In embodiments, the therapeutically effective amount may be administered three times per day for about 28 days to about 24 weeks.

The present disclosure also describes a method of producing reversible infertility in a male subject, the method including administering to the male subject comprising administering to the male subject a therapeutically effective amount of a compound of formula (I):

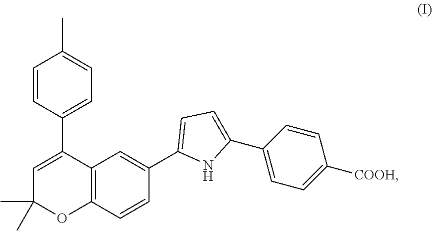 [SMILES: Cc1ccc(C2=CC(C)(C)Oc3ccc(-c4ccc(-c5ccc(C(=O)O)cc5)[nH]4)cc32)cc1]

or a pharmaceutically acceptable salt thereof, wherein the therapeutically effective amount is about 15 mg to about 450 mg.

In embodiments, the therapeutically effective amount to produce reversible infertility in a male subject may be about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, or any value contained within a range formed by any two of the preceding values.

For example, in embodiments, the therapeutically effective amount may be about 15 mg to about 180 mg, about 15 mg to about 150 mg, about 15 mg to about 100 mg, about 15 mg to about 80 mg, about 15 mg to about 50 mg, or any value contained within a range formed by any two of the preceding values. In embodiments, the therapeutically effective amount may be about 180 mg to about 450 mg, about 200 mg to about 450 mg, about 250 mg to about 450 mg, about 300 mg to about 450 mg, about 350 mg to about 450 mg, or any value contained within a range formed by any two of the preceding values.

In embodiments, the therapeutically effective amount to produce reversible infertility in a male subject may be about 15 mg. In embodiments, the therapeutically effective amount to produce reversible infertility in a male subject may be about 20 mg. In embodiments, the therapeutically effective amount to produce reversible infertility in a male subject may be about 25 mg. In embodiments, the therapeutically effective amount to produce reversible infertility in a male subject may be about 30 mg. In embodiments, the therapeutically effective amount to produce reversible infertility in a male subject may be about 35 mg. In embodiments, the therapeutically effective amount to produce reversible infertility in a male subject may be about 40 mg. In embodiments, the therapeutically effective amount to produce reversible infertility in a male subject may be about 45 mg. In embodiments, the therapeutically effective amount to produce reversible infertility in a male subject may be about 50 mg. In embodiments, the therapeutically effective amount to produce reversible infertility in a male subject may be about 55 mg. In embodiments, the therapeutically effective amount to produce reversible infertility in a male subject may be about 60 mg. In embodiments, the therapeutically effective amount to produce reversible infertility in a male subject may be about 65 mg. In embodiments, the therapeutically effective amount to produce reversible infertility in a male subject may be about 70 mg. In embodiments, the therapeutically effective amount to produce reversible infertility in a male subject may be about 75 mg. In embodiments, the therapeutically effective amount to produce reversible infertility in a male subject may be about 80 mg. In embodiments, the therapeutically effective amount to produce reversible infertility in a male subject may be about 85 mg. In embodiments, the therapeutically effective amount to produce reversible infertility in a male subject may be about 90 mg. In embodiments, the therapeutically effective amount to produce reversible infertility in a male subject may be about 95 mg. In embodiments, the therapeutically effective amount to produce reversible infertility in a male subject may be about 100 mg. In embodiments, the therapeutically effective amount to produce reversible infertility in a male subject may be about 105 mg. In embodiments, the therapeutically effective amount to produce reversible infertility in a male subject may be about 110 mg. In embodiments, the therapeutically effective amount to produce reversible infertility in a male subject may be about 115 mg. In embodiments, the therapeutically effective amount to produce reversible infertility in a male subject may be about 120 mg. In embodiments, the therapeutically effective amount to produce reversible infertility in a male subject may be about 125 mg. In embodiments, the therapeutically effective amount to produce reversible infertility in a male subject may be about 130 mg. In embodiments, the therapeutically effective amount to produce reversible infertility in a male subject may be about 135 mg. In embodiments, the therapeutically effective amount to produce reversible infertility in a male subject may be about 140 mg. In embodiments, the therapeutically effective amount to produce reversible infertility in a male subject may be about 145 mg. In embodiments, the therapeutically effective amount to produce reversible infertility in a male subject may be about 150 mg. In embodiments, the therapeutically effective amount to produce reversible infertility in a male subject may be about 155 mg. In embodiments, the therapeutically effective amount to produce reversible infertility in a male subject may be about 160 mg. In embodiments, the therapeutically effective amount to produce reversible infertility in a male subject may be about 165 mg. In embodiments, the therapeutically effective amount to produce reversible infertility in a male subject may be about 170 mg. In embodiments, the therapeutically effective amount to produce reversible infertility in a male subject may be about 175 mg. In embodiments, the therapeutically effective amount to produce reversible infertility in a male subject may be about 180 mg. In embodiments, the therapeutically effective amount to produce reversible infertility in a male subject may be about 185 mg. In embodiments, the therapeutically effective amount to produce reversible infertility in a male subject may be about 190 mg. In embodiments, the therapeutically effective amount to produce reversible infertility in a male subject may be about 195 mg. In embodiments, the therapeutically effective amount to produce reversible infertility in a male subject may be about 200 mg. In embodiments, the therapeutically effective amount to produce reversible infertility in a male subject may be about 205 mg. In embodiments, the therapeutically effective amount to produce reversible infertility in a male subject may be about 210 mg. In embodiments, the therapeutically effective amount to produce reversible infertility in a male subject may be about 215 mg. In embodiments, the therapeutically effective amount to produce reversible infertility in a male subject may be about 220 mg. In embodiments, the therapeutically effective amount to produce reversible infertility in a male subject may be about 225 mg. In embodiments, the therapeutically effective amount to produce reversible infertility in a male subject may be about 230 mg. In embodiments, the therapeutically effective amount to produce reversible infertility in a male subject may be about 235 mg. In embodiments, the therapeutically effective amount to produce reversible infertility in a male subject may be about 240 mg. In embodiments, the therapeutically effective amount to produce reversible infertility in a male subject may be about 245 mg. In embodiments, the therapeutically effective amount to produce reversible infertility in a male subject may be about 250 mg. In embodiments, the therapeutically effective amount to produce reversible infertility in a male subject may be about 255 mg. In embodiments, the therapeutically effective amount to produce reversible infertility in a male subject may be about 260 mg. In embodiments, the therapeutically effective amount to produce reversible infertility in a male subject may be about 265 mg. In embodiments, the therapeutically effective amount to produce reversible infertility in a male subject may be about 270 mg. In embodiments, the therapeutically effective amount to produce reversible infertility in a male subject may be about 275 mg. In embodiments, the therapeutically effective amount to produce reversible infertility in a male subject may be about 280 mg. In embodiments, the therapeutically effective amount to produce reversible infertility in a male subject may be about 285 mg. In embodiments, the therapeutically effective amount to produce reversible infertility in a male subject may be about 290 mg. In embodiments, the therapeutically effective amount to produce reversible infertility in a male subject may be about 295 mg. In embodiments, the therapeutically effective amount to produce reversible infertility in a male subject may be about 300 mg. In embodiments, the therapeutically effective amount to produce reversible infertility in a male subject may be about 305 mg. In embodiments, the therapeutically effective amount to produce reversible infertility in a male subject may be about 310 mg. In embodiments, the therapeutically effective amount to produce reversible infertility in a male subject may be about 315 mg. In embodiments, the therapeutically effective amount to produce reversible infertility in a male subject may be about 320 mg. In embodiments, the therapeutically effective amount to produce reversible infertility in a male subject may be about 325 mg. In embodiments, the therapeutically effective amount to produce reversible infertility in a male subject may be about 330 mg. In embodiments, the therapeutically effective amount to produce reversible infertility in a male subject may be about 335 mg. In embodiments, the therapeutically effective amount to produce reversible infertility in a male subject may be about 340 mg. In embodiments, the therapeutically effective amount to produce reversible infertility in a male subject may be about 345 mg. In embodiments, the therapeutically effective amount to produce reversible infertility in a male subject may be about 350 mg. In embodiments, the therapeutically effective amount to produce reversible infertility in a male subject may be about 355 mg. In embodiments, the therapeutically effective amount to produce reversible infertility in a male subject may be about 360 mg. In embodiments, the therapeutically effective amount to produce reversible infertility in a male subject may be about 365 mg. In embodiments, the therapeutically effective amount to produce reversible infertility in a male subject may be about 370 mg. In embodiments, the therapeutically effective amount to produce reversible infertility in a male subject may be about 375 mg. In embodiments, the therapeutically effective amount to produce reversible infertility in a male subject may be about 380 mg. In embodiments, the therapeutically effective amount to produce reversible infertility in a male subject may be about 385 mg. In embodiments, the therapeutically effective amount to produce reversible infertility in a male subject may be about 390 mg. In embodiments, the therapeutically effective amount to produce reversible infertility in a male subject may be about 395 mg. In embodiments, the therapeutically effective amount to produce reversible infertility in a male subject may be about 400 mg. In embodiments, the therapeutically effective amount to produce reversible infertility in a male subject may be about 405 mg. In embodiments, the therapeutically effective amount to produce reversible infertility in a male subject may be about 410 mg. In embodiments, the therapeutically effective amount to produce reversible infertility in a male subject may be about 415 mg. In embodiments, the therapeutically effective amount to produce reversible infertility in a male subject may be about 420 mg. In embodiments, the therapeutically effective amount to produce reversible infertility in a male subject may be about 425 mg. In embodiments, the therapeutically effective amount to produce reversible infertility in a male subject may be about 430 mg. In embodiments, the therapeutically effective amount to produce reversible infertility in a male subject may be about 435 mg. In embodiments, the therapeutically effective amount to produce reversible infertility in a male subject may be about 440 mg. In embodiments, the therapeutically effective amount to produce reversible infertility in a male subject may be about 445 mg. In embodiments, the therapeutically effective amount to produce reversible infertility in a male subject may be about 450 mg.

In embodiments, the therapeutically effective amount is administered once per day for about 28 days to about 24 weeks, such as about 28 days, about 29 days, about 30 days, about 31 days, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, or any value contained within a range formed by any two of the preceding values.

For example, in embodiments, the therapeutically effective amount is administered once per day for about 13 weeks to about 24 weeks, about 13 weeks to about 20 weeks, about 16 weeks to about 24 weeks, about 20 weeks to about 24 weeks, or any value contained within a range formed by any two of the preceding values. In embodiments, the therapeutically effective amount is administered once per day for about 13 weeks. In embodiments, the therapeutically effective amount is administered once per day for about 14 weeks. In embodiments, the therapeutically effective amount is administered once per day for about 15 weeks. In embodiments, the therapeutically effective amount is administered once per day for about 16 weeks. In embodiments, the therapeutically effective amount is administered once per day for about 17 weeks. In embodiments, the therapeutically effective amount is administered once per day for about 18 weeks. In embodiments, the therapeutically effective amount is administered once per day for about 19 weeks. In embodiments, the therapeutically effective amount is administered once per day for about 20 weeks. In embodiments, the therapeutically effective amount is administered once per day for about 21 weeks. In embodiments, the therapeutically effective amount is administered once per day for about 22 weeks. In embodiments, the therapeutically effective amount is administered once per day for about 23 weeks. In embodiments, the therapeutically effective amount is administered once per day for about 24 weeks.

In embodiments, the therapeutically effective amount is about 15 mg and the therapeutically effective amount is administered once per day for about 13 weeks. In embodiments, the therapeutically effective amount is about 30 mg and the therapeutically effective amount is administered once per day for about 13 weeks. In embodiments, the therapeutically effective amount is about 45 mg and the therapeutically effective amount is administered once per day for about 13 weeks. In embodiments, the therapeutically effective amount is about 90 mg and the therapeutically effective amount is administered once per day for about 13 weeks. In embodiments, the therapeutically effective amount is about 180 mg and the therapeutically effective amount is administered once per day for about 13 weeks. In embodiments, the therapeutically effective amount is about 270 mg and the therapeutically effective amount is administered once per day for about 13 weeks. In embodiments, the therapeutically effective amount is about 300 mg and the therapeutically effective amount is administered once per day for about 13 weeks. In embodiments, the therapeutically effective amount is about 360 mg and the therapeutically effective amount is administered once per day for about 13 weeks. In embodiments, the therapeutically effective amount is about 450 mg and the therapeutically effective amount is administered once per day for about 13 weeks.

In embodiments, the therapeutically effective amount is about 15 mg and the therapeutically effective amount is administered once per day for about 20 weeks. In embodiments, the therapeutically effective amount is about 30 mg and the therapeutically effective amount is administered once per day for about 20 weeks. In embodiments, the therapeutically effective amount is about 45 mg and the therapeutically effective amount is administered once per day for about 20 weeks. In embodiments, the therapeutically effective amount is about 90 mg and the therapeutically effective amount is administered once per day for about 20 weeks. In embodiments, the therapeutically effective amount is about 180 mg and the therapeutically effective amount is administered once per day for about 20 weeks. In embodiments, the therapeutically effective amount is about 270 mg and the therapeutically effective amount is administered once per day for about 20 weeks. In embodiments, the therapeutically effective amount is about 300 mg and the therapeutically effective amount is administered once per day for about 20 weeks. In embodiments, the therapeutically effective amount is about 360 mg and the therapeutically effective amount is administered once per day for about 20 weeks. In embodiments, the therapeutically effective amount is about 450 mg and the therapeutically effective amount is administered once per day for about 20 weeks.

In embodiments, the therapeutically effective amount is about 15 mg and the therapeutically effective amount is administered once per day for about 24 weeks. In embodiments, the therapeutically effective amount is about 30 mg and the therapeutically effective amount is administered once per day for about 24 weeks. In embodiments, the therapeutically effective amount is about 45 mg and the therapeutically effective amount is administered once per day for about 24 weeks. In embodiments, the therapeutically effective amount is about 90 mg and the therapeutically effective amount is administered once per day for about 24 weeks. In embodiments, the therapeutically effective amount is about 180 mg and the therapeutically effective amount is administered once per day for about 24 weeks. In embodiments, the therapeutically effective amount is about 270 mg and the therapeutically effective amount is administered once per day for about 24 weeks. In embodiments, the therapeutically effective amount is about 300 mg and the therapeutically effective amount is administered once per day for about 24 weeks. In embodiments, the therapeutically effective amount is about 360 mg and the therapeutically effective amount is administered once per day for about 24 weeks. In embodiments, the therapeutically effective amount is about 450 mg and the therapeutically effective amount is administered once per day for about 24 weeks.

In embodiments, the therapeutically effective amount may be administered once per day, twice per day, or three times per day. In embodiments, the therapeutically effective amount may be administered twice per day for about 28 days to about 24 weeks. In embodiments, the therapeutically effective amount may be administered three times per day for about 28 days to about 24 weeks.

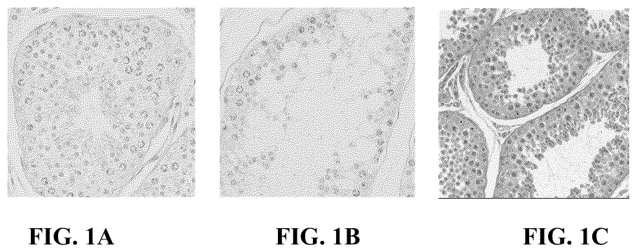

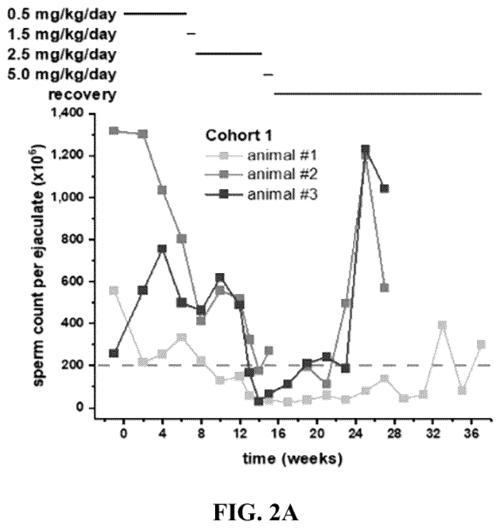

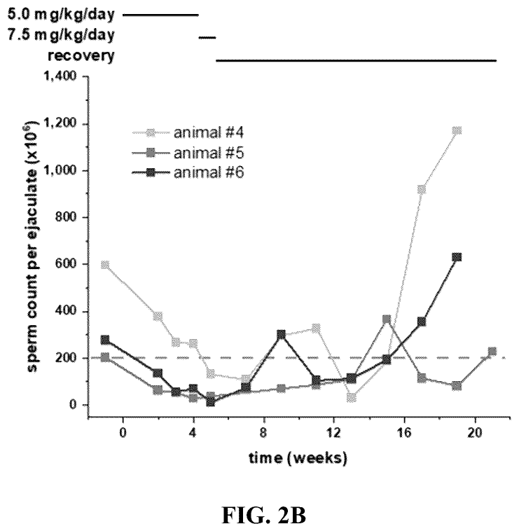

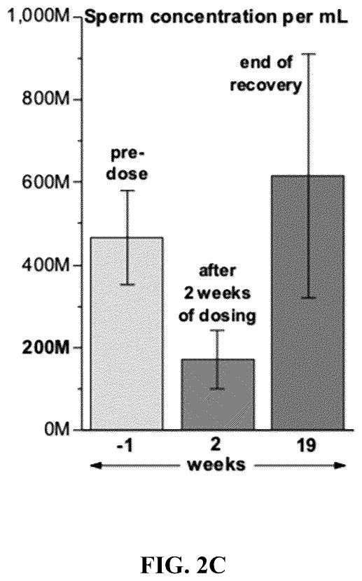

FIG. 1A is a cross-section of a canine testicle prior to dosing with the compound of the present disclosure, showing developing and mature sperm. FIG. 1B is a cross-section of a canine testicle after dosing with the compound of the present disclosure, showing no developing and mature sperm. FIG. 1C is a cross-section of a canine testicle after a recovery period of no dosing with the compound of the present disclosure, showing the return of developing sperm. FIGS. 1A-1C demonstrate the efficacy of the compound and method of the present disclosure of producing reversible infertility in a male subject.

The present disclosure also provides a method of reducing sperm count in a male subject, the method including administering to the male subject a therapeutically effective amount of a compound of formula (I):

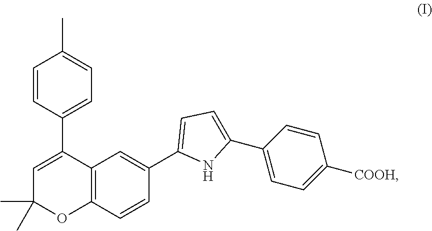 [SMILES: Cc1ccc(C2=CC(C)(C)Oc3ccc(-c4ccc(-c5ccc(C(=O)O)cc5)[nH]4)cc32)cc1]

or a pharmaceutically acceptable salt thereof, wherein the therapeutically effective amount is about 15 mg to about 450 mg.

In embodiments, the therapeutically effective amount to reduce sperm count in a male subject may be about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, or any value contained within a range formed by any two of the preceding values.

For example, in embodiments, the therapeutically effective amount may be about 15 mg to about 180 mg, about 15 mg to about 150 mg, about 15 mg to about 100 mg, about 15 mg to about 80 mg, about 15 mg to about 50 mg, or any value contained within a range formed by any two of the preceding values. In embodiments, the therapeutically effective amount may be about 180 mg to about 450 mg, about 200 mg to about 450 mg, about 250 mg to about 450 mg, about 300 mg to about 450 mg, about 350 mg to about 450 mg, or any value contained within a range formed by any two of the preceding values.

In embodiments, the therapeutically effective amount to reduce sperm count in a male subject may be about 15 mg. In embodiments, the therapeutically effective amount to reduce sperm count in a male subject may be about 20 mg. In embodiments, the therapeutically effective amount to reduce sperm count in a male subject may be about 25 mg. In embodiments, the therapeutically effective amount to reduce sperm count in a male subject may be about 30 mg. In embodiments, the therapeutically effective amount to reduce sperm count in a male subject may be about 35 mg. In embodiments, the therapeutically effective amount to reduce sperm count in a male subject may be about 40 mg. In embodiments, the therapeutically effective amount to reduce sperm count in a male subject may be about 45 mg. In embodiments, the therapeutically effective amount to reduce sperm count in a male subject may be about 50 mg. In embodiments, the therapeutically effective amount to reduce sperm count in a male subject may be about 55 mg. In embodiments, the therapeutically effective amount to reduce sperm count in a male subject may be about 60 mg. In embodiments, the therapeutically effective amount to reduce sperm count in a male subject may be about 65 mg. In embodiments, the therapeutically effective amount to reduce sperm count in a male subject may be about 70 mg. In embodiments, the therapeutically effective amount to reduce sperm count in a male subject may be about 75 mg. In embodiments, the therapeutically effective amount to reduce sperm count in a male subject may be about 80 mg. In embodiments, the therapeutically effective amount to reduce sperm count in a male subject may be about 85 mg. In embodiments, the therapeutically effective amount to reduce sperm count in a male subject may be about 90 mg. In embodiments, the therapeutically effective amount to reduce sperm count in a male subject may be about 95 mg. In embodiments, the therapeutically effective amount to reduce sperm count in a male subject may be about 100 mg. In embodiments, the therapeutically effective amount to reduce sperm count in a male subject may be about 105 mg. In embodiments, the therapeutically effective amount to reduce sperm count in a male subject may be about 110 mg. In embodiments, the therapeutically effective amount to reduce sperm count in a male subject may be about 115 mg. In embodiments, the therapeutically effective amount to reduce sperm count in a male subject may be about 120 mg. In embodiments, the therapeutically effective amount to reduce sperm count in a male subject may be about 125 mg. In embodiments, the therapeutically effective amount to reduce sperm count in a male subject may be about 130 mg. In embodiments, the therapeutically effective amount to reduce sperm count in a male subject may be about 135 mg. In embodiments, the therapeutically effective amount to reduce sperm count in a male subject may be about 140 mg. In embodiments, the therapeutically effective amount to reduce sperm count in a male subject may be about 145 mg. In embodiments, the therapeutically effective amount to reduce sperm count in a male subject may be about 150 mg. In embodiments, the therapeutically effective amount to reduce sperm count in a male subject may be about 155 mg. In embodiments, the therapeutically effective amount to reduce sperm count in a male subject may be about 160 mg. In embodiments, the therapeutically effective amount to reduce sperm count in a male subject may be about 165 mg. In embodiments, the therapeutically effective amount to reduce sperm count in a male subject may be about 170 mg. In embodiments, the therapeutically effective amount to reduce sperm count in a male subject may be about 175 mg. In embodiments, the therapeutically effective amount to reduce sperm count in a male subject may be about 180 mg. In embodiments, the therapeutically effective amount to reduce sperm count in a male subject may be about 185 mg. In embodiments, the therapeutically effective amount to reduce sperm count in a male subject may be about 190 mg. In embodiments, the therapeutically effective amount to reduce sperm count in a male subject may be about 195 mg. In embodiments, the therapeutically effective amount to reduce sperm count in a male subject may be about 200 mg. In embodiments, the therapeutically effective amount to reduce sperm count in a male subject may be about 205 mg. In embodiments, the therapeutically effective amount to reduce sperm count in a male subject may be about 210 mg. In embodiments, the therapeutically effective amount to reduce sperm count in a male subject may be about 215 mg. In embodiments, the therapeutically effective amount to reduce sperm count in a male subject may be about 220 mg. In embodiments, the therapeutically effective amount to reduce sperm count in a male subject may be about 225 mg. In embodiments, the therapeutically effective amount to reduce sperm count in a male subject may be about 230 mg. In embodiments, the therapeutically effective amount to reduce sperm count in a male subject may be about 235 mg. In embodiments, the therapeutically effective amount to reduce sperm count in a male subject may be about 240 mg. In embodiments, the therapeutically effective amount to reduce sperm count in a male subject may be about 245 mg. In embodiments, the therapeutically effective amount to reduce sperm count in a male subject may be about 250 mg. In embodiments, the therapeutically effective amount to reduce sperm count in a male subject may be about 255 mg. In embodiments, the therapeutically effective amount to reduce sperm count in a male subject may be about 260 mg. In embodiments, the therapeutically effective amount to reduce sperm count in a male subject may be about 265 mg. In embodiments, the therapeutically effective amount to reduce sperm count in a male subject may be about 270 mg. In embodiments, the therapeutically effective amount to reduce sperm count in a male subject may be about 275 mg. In embodiments, the therapeutically effective amount to reduce sperm count in a male subject may be about 280 mg. In embodiments, the therapeutically effective amount to reduce sperm count in a male subject may be about 285 mg. In embodiments, the therapeutically effective amount to reduce sperm count in a male subject may be about 290 mg. In embodiments, the therapeutically effective amount to reduce sperm count in a male subject may be about 295 mg. In embodiments, the therapeutically effective amount to reduce sperm count in a male subject may be about 300 mg. In embodiments, the therapeutically effective amount to reduce sperm count in a male subject may be about 305 mg. In embodiments, the therapeutically effective amount to reduce sperm count in a male subject may be about 310 mg. In embodiments, the therapeutically effective amount to reduce sperm count in a male subject may be about 315 mg. In embodiments, the therapeutically effective amount to reduce sperm count in a male subject may be about 320 mg. In embodiments, the therapeutically effective amount to reduce sperm count in a male subject may be about 325 mg. In embodiments, the therapeutically effective amount to reduce sperm count in a male subject may be about 330 mg. In embodiments, the therapeutically effective amount to reduce sperm count in a male subject may be about 335 mg. In embodiments, the therapeutically effective amount to reduce sperm count in a male subject may be about 340 mg. In embodiments, the therapeutically effective amount to reduce sperm count in a male subject may be about 345 mg. In embodiments, the therapeutically effective amount to reduce sperm count in a male subject may be about 350 mg. In embodiments, the therapeutically effective amount to reduce sperm count in a male subject may be about 355 mg. In embodiments, the therapeutically effective amount to reduce sperm count in a male subject may be about 360 mg. In embodiments, the therapeutically effective amount to reduce sperm count in a male subject may be about 365 mg. In embodiments, the therapeutically effective amount to reduce sperm count in a male subject may be about 370 mg. In embodiments, the therapeutically effective amount to reduce sperm count in a male subject may be about 375 mg. In embodiments, the therapeutically effective amount to reduce sperm count in a male subject may be about 380 mg. In embodiments, the therapeutically effective amount to reduce sperm count in a male subject may be about 385 mg. In embodiments, the therapeutically effective amount to reduce sperm count in a male subject may be about 390 mg. In embodiments, the therapeutically effective amount to reduce sperm count in a male subject may be about 395 mg. In embodiments, the therapeutically effective amount to reduce sperm count in a male subject may be about 400 mg. In embodiments, the therapeutically effective amount to reduce sperm count in a male subject may be about 405 mg. In embodiments, the therapeutically effective amount to reduce sperm count in a male subject may be about 410 mg. In embodiments, the therapeutically effective amount to reduce sperm count in a male subject may be about 415 mg. In embodiments, the therapeutically effective amount to reduce sperm count in a male subject may be about 420 mg. In embodiments, the therapeutically effective amount to reduce sperm count in a male subject may be about 425 mg. In embodiments, the therapeutically effective amount to reduce sperm count in a male subject may be about 430 mg. In embodiments, the therapeutically effective amount to reduce sperm count in a male subject may be about 435 mg. In embodiments, the therapeutically effective amount to reduce sperm count in a male subject may be about 440 mg. In embodiments, the therapeutically effective amount to reduce sperm count in a male subject may be about 445 mg. In embodiments, the therapeutically effective amount to reduce sperm count in a male subject may be about 450 mg.

In embodiments, the therapeutically effective amount is administered once per day for about 28 days to about 24 weeks, such as about 28 days, about 29 days, about 30 days, about 31 days, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, or any value contained within a range formed by any two of the preceding values.

For example, in embodiments, the therapeutically effective amount is administered once per day for about 13 weeks to about 24 weeks, about 13 weeks to about 20 weeks, about 16 weeks to about 24 weeks, about 20 weeks to about 24 weeks, or any value contained within a range formed by any two of the preceding values. In embodiments, the therapeutically effective amount is administered once per day for about 13 weeks. In embodiments, the therapeutically effective amount is administered once per day for about 14 weeks. In embodiments, the therapeutically effective amount is administered once per day for about 15 weeks. In embodiments, the therapeutically effective amount is administered once per day for about 16 weeks. In embodiments, the therapeutically effective amount is administered once per day for about 17 weeks. In embodiments, the therapeutically effective amount is administered once per day for about 18 weeks. In embodiments, the therapeutically effective amount is administered once per day for about 19 weeks. In embodiments, the therapeutically effective amount is administered once per day for about 20 weeks. In embodiments, the therapeutically effective amount is administered once per day for about 21 weeks. In embodiments, the therapeutically effective amount is administered once per day for about 22 weeks. In embodiments, the therapeutically effective amount is administered once per day for about 23 weeks. In embodiments, the therapeutically effective amount is administered once per day for about 24 weeks.

In embodiments, the therapeutically effective amount is about 15 mg and the therapeutically effective amount is administered once per day for about 13 weeks. In embodiments, the therapeutically effective amount is about 30 mg and the therapeutically effective amount is administered once per day for about 13 weeks. In embodiments, the therapeutically effective amount is about 45 mg and the therapeutically effective amount is administered once per day for about 13 weeks. In embodiments, the therapeutically effective amount is about 90 mg and the therapeutically effective amount is administered once per day for about 13 weeks. In embodiments, the therapeutically effective amount is about 180 mg and the therapeutically effective amount is administered once per day for about 13 weeks. In embodiments, the therapeutically effective amount is about 270 mg and the therapeutically effective amount is administered once per day for about 13 weeks. In embodiments, the therapeutically effective amount is about 300 mg and the therapeutically effective amount is administered once per day for about 13 weeks. In embodiments, the therapeutically effective amount is about 360 mg and the therapeutically effective amount is administered once per day for about 13 weeks. In embodiments, the therapeutically effective amount is about 450 mg and the therapeutically effective amount is administered once per day for about 13 weeks.

In embodiments, the therapeutically effective amount is about 15 mg and the therapeutically effective amount is administered once per day for about 20 weeks. In embodiments, the therapeutically effective amount is about 30 mg and the therapeutically effective amount is administered once per day for about 20 weeks. In embodiments, the therapeutically effective amount is about 45 mg and the therapeutically effective amount is administered once per day for about 20 weeks. In embodiments, the therapeutically effective amount is about 90 mg and the therapeutically effective amount is administered once per day for about 20 weeks. In embodiments, the therapeutically effective amount is about 180 mg and the therapeutically effective amount is administered once per day for about 20 weeks. In embodiments, the therapeutically effective amount is about 270 mg and the therapeutically effective amount is administered once per day for about 20 weeks. In embodiments, the therapeutically effective amount is about 300 mg and the therapeutically effective amount is administered once per day for about 20 weeks. In embodiments, the therapeutically effective amount is about 360 mg and the therapeutically effective amount is administered once per day for about 20 weeks. In embodiments, the therapeutically effective amount is about 450 mg and the therapeutically effective amount is administered once per day for about 20 weeks.

In embodiments, the therapeutically effective amount is about 15 mg and the therapeutically effective amount is administered once per day for about 24 weeks. In embodiments, the therapeutically effective amount is about 30 mg and the therapeutically effective amount is administered once per day for about 24 weeks. In embodiments, the therapeutically effective amount is about 45 mg and the therapeutically effective amount is administered once per day for about 24 weeks. In embodiments, the therapeutically effective amount is about 90 mg and the therapeutically effective amount is administered once per day for about 24 weeks. In embodiments, the therapeutically effective amount is about 180 mg and the therapeutically effective amount is administered once per day for about 24 weeks. In embodiments, the therapeutically effective amount is about 270 mg and the therapeutically effective amount is administered once per day for about 24 weeks. In embodiments, the therapeutically effective amount is about 300 mg and the therapeutically effective amount is administered once per day for about 24 weeks. In embodiments, the therapeutically effective amount is about 360 mg and the therapeutically effective amount is administered once per day for about 24 weeks. In embodiments, the therapeutically effective amount is about 450 mg and the therapeutically effective amount is administered once per day for about 24 weeks.

In embodiments, the therapeutically effective amount may be administered once per day, twice per day, or three times per day. In embodiments, the therapeutically effective amount may be administered twice per day for about 28 days to about 24 weeks. In embodiments, the therapeutically effective amount may be administered three times per day for about 28 days to about 24 weeks.

The present disclosure also provides a method of selectively antagonizing RAR alpha over RAR beta and RAR gamma in a subject, the method including administering to the subject a therapeutically effective amount of a compound of formula (I):

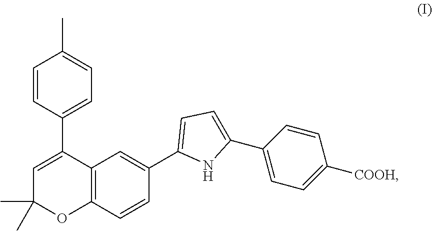 [SMILES: Cc1ccc(C2=CC(C)(C)Oc3ccc(-c4ccc(-c5ccc(C(=O)O)cc5)[nH]4)cc32)cc1]

or a pharmaceutically acceptable salt thereof, wherein the therapeutically effective amount is about 15 mg to about 450 mg.

In embodiments, the therapeutically effective amount to selectively antagonize RAR alpha over RAR beta and RAR gamma in a subject may be about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, or any value contained within a range formed by any two of the preceding values.

For example, in embodiments, the therapeutically effective amount may be about 15 mg to about 180 mg, about 15 mg to about 150 mg, about 15 mg to about 100 mg, about 15 mg to about 80 mg, about 15 mg to about 50 mg, or any value contained within a range formed by any two of the preceding values. In embodiments, the therapeutically effective amount may be about 180 mg to about 450 mg, about 200 mg to about 450 mg, about 250 mg to about 450 mg, about 300 mg to about 450 mg, about 350 mg to about 450 mg, or any value contained within a range formed by any two of the preceding values.

In embodiments, the therapeutically effective amount to selectively antagonize RAR alpha over RAR beta and RAR gamma in a subject may be about 15 mg. In embodiments, the therapeutically effective amount to selectively antagonize RAR alpha over RAR beta and RAR gamma in a subject may be about 20 mg. In embodiments, the therapeutically effective amount to selectively antagonize RAR alpha over RAR beta and RAR gamma in a subject may be about 25 mg. In embodiments, the therapeutically effective amount to selectively antagonize RAR alpha over RAR beta and RAR gamma in a subject may be about 30 mg. In embodiments, the therapeutically effective amount to selectively antagonize RAR alpha over RAR beta and RAR gamma in a subject may be about 35 mg. In embodiments, the therapeutically effective amount to selectively antagonize RAR alpha over RAR beta and RAR gamma in a subject may be about 40 mg. In embodiments, the therapeutically effective amount to selectively antagonize RAR alpha over RAR beta and RAR gamma in a subject may be about 45 mg. In embodiments, the therapeutically effective amount to selectively antagonize RAR alpha over RAR beta and RAR gamma in a subject may be about 50 mg. In embodiments, the therapeutically effective amount to selectively antagonize RAR alpha over RAR beta and RAR gamma in a subject may be about 55 mg. In embodiments, the therapeutically effective amount to selectively antagonize RAR alpha over RAR beta and RAR gamma in a subject may be about 60 mg. In embodiments, the therapeutically effective amount to selectively antagonize RAR alpha over RAR beta and RAR gamma in a subject may be about 65 mg. In embodiments, the therapeutically effective amount to selectively antagonize RAR alpha over RAR beta and RAR gamma in a subject may be about 70 mg. In embodiments, the therapeutically effective amount to selectively antagonize RAR alpha over RAR beta and RAR gamma in a subject may be about 75 mg. In embodiments, the therapeutically effective amount to selectively antagonize RAR alpha over RAR beta and RAR gamma in a subject may be about 80 mg. In embodiments, the therapeutically effective amount to selectively antagonize RAR alpha over RAR beta and RAR gamma in a subject may be about 85 mg. In embodiments, the therapeutically effective amount to selectively antagonize RAR alpha over RAR beta and RAR gamma in a subject may be about 90 mg. In embodiments, the therapeutically effective amount to selectively antagonize RAR alpha over RAR beta and RAR gamma in a subject may be about 95 mg. In embodiments, the therapeutically effective amount to selectively antagonize RAR alpha over RAR beta and RAR gamma in a subject may be about 100 mg. In embodiments, the therapeutically effective amount to selectively antagonize RAR alpha over RAR beta and RAR gamma in a subject may be about 105 mg. In embodiments, the therapeutically effective amount to selectively antagonize RAR alpha over RAR beta and RAR gamma in a subject may be about 110 mg. In embodiments, the therapeutically effective amount to selectively antagonize RAR alpha over RAR beta and RAR gamma in a subject may be about 115 mg. In embodiments, the therapeutically effective amount to selectively antagonize RAR alpha over RAR beta and RAR gamma in a subject may be about 120 mg. In embodiments, the therapeutically effective amount to selectively antagonize RAR alpha over RAR beta and RAR gamma in a subject may be about 125 mg. In embodiments, the therapeutically effective amount to selectively antagonize RAR alpha over RAR beta and RAR gamma in a subject may be about 130 mg. In embodiments, the therapeutically effective amount to selectively antagonize RAR alpha over RAR beta and RAR gamma in a subject may be about 135 mg. In embodiments, the therapeutically effective amount to selectively antagonize RAR alpha over RAR beta and RAR gamma in a subject may be about 140 mg. In embodiments, the therapeutically effective amount to selectively antagonize RAR alpha over RAR beta and RAR gamma in a subject may be about 145 mg. In embodiments, the therapeutically effective amount to selectively antagonize RAR alpha over RAR beta and RAR gamma in a subject may be about 150 mg. In embodiments, the therapeutically effective amount to selectively antagonize RAR alpha over RAR beta and RAR gamma in a subject may be about 155 mg. In embodiments, the therapeutically effective amount to selectively antagonize RAR alpha over RAR beta and RAR gamma in a subject may be about 160 mg. In embodiments, the therapeutically effective amount to selectively antagonize RAR alpha over RAR beta and RAR gamma in a subject may be about 165 mg. In embodiments, the therapeutically effective amount to selectively antagonize RAR alpha over RAR beta and RAR gamma in a subject may be about 170 mg. In embodiments, the therapeutically effective amount to selectively antagonize RAR alpha over RAR beta and RAR gamma in a subject may be about 175 mg. In embodiments, the therapeutically effective amount to selectively antagonize RAR alpha over RAR beta and RAR gamma in a subject may be about 180 mg. In embodiments, the therapeutically effective amount to selectively antagonize RAR alpha over RAR beta and RAR gamma in a subject may be about 185 mg. In embodiments, the therapeutically effective amount to selectively antagonize RAR alpha over RAR beta and RAR gamma in a subject may be about 190 mg. In embodiments, the therapeutically effective amount to selectively antagonize RAR alpha over RAR beta and RAR gamma in a subject may be about 195 mg. In embodiments, the therapeutically effective amount to selectively antagonize RAR alpha over RAR beta and RAR gamma in a subject may be about 200 mg. In embodiments, the therapeutically effective amount to selectively antagonize RAR alpha over RAR beta and RAR gamma in a subject may be about 205 mg. In embodiments, the therapeutically effective amount to selectively antagonize RAR alpha over RAR beta and RAR gamma in a subject may be about 210 mg. In embodiments, the therapeutically effective amount to selectively antagonize RAR alpha over RAR beta and RAR gamma in a subject may be about 215 mg. In embodiments, the therapeutically effective amount to selectively antagonize RAR alpha over RAR beta and RAR gamma in a subject may be about 220 mg. In embodiments, the therapeutically effective amount to selectively antagonize RAR alpha over RAR beta and RAR gamma in a subject may be about 225 mg. In embodiments, the therapeutically effective amount to selectively antagonize RAR alpha over RAR beta and RAR gamma in a subject may be about 230 mg. In embodiments, the therapeutically effective amount to selectively antagonize RAR alpha over RAR beta and RAR gamma in a subject may be about 235 mg. In embodiments, the therapeutically effective amount to selectively antagonize RAR alpha over RAR beta and RAR gamma in a subject may be about 240 mg. In embodiments, the therapeutically effective amount to selectively antagonize RAR alpha over RAR beta and RAR gamma in a subject may be about 245 mg. In embodiments, the therapeutically effective amount to selectively antagonize RAR alpha over RAR beta and RAR gamma in a subject may be about 250 mg. In embodiments, the therapeutically effective amount to selectively antagonize RAR alpha over RAR beta and RAR gamma in a subject may be about 255 mg. In embodiments, the therapeutically effective amount to selectively antagonize RAR alpha over RAR beta and RAR gamma in a subject may be about 260 mg. In embodiments, the therapeutically effective amount to selectively antagonize RAR alpha over RAR beta and RAR gamma in a subject may be about 265 mg. In embodiments, the therapeutically effective amount to selectively antagonize RAR alpha over RAR beta and RAR gamma in a subject may be about 270 mg. In embodiments, the therapeutically effective amount to selectively antagonize RAR alpha over RAR beta and RAR gamma in a subject may be about 275 mg. In embodiments, the therapeutically effective amount to selectively antagonize RAR alpha over RAR beta and RAR gamma in a subject may be about 280 mg. In embodiments, the therapeutically effective amount to selectively antagonize RAR alpha over RAR beta and RAR gamma in a subject may be about 285 mg. In embodiments, the therapeutically effective amount to selectively antagonize RAR alpha over RAR beta and RAR gamma in a subject may be about 290 mg. In embodiments, the therapeutically effective amount to selectively antagonize RAR alpha over RAR beta and RAR gamma in a subject may be about 295 mg. In embodiments, the therapeutically effective amount to selectively antagonize RAR alpha over RAR beta and RAR gamma in a subject may be about 300 mg. In embodiments, the therapeutically effective amount to selectively antagonize RAR alpha over RAR beta and RAR gamma in a subject may be about 305 mg. In embodiments, the therapeutically effective amount to selectively antagonize RAR alpha over RAR beta and RAR gamma in a subject may be about 310 mg. In embodiments, the therapeutically effective amount to selectively antagonize RAR alpha over RAR beta and RAR gamma in a subject may be about 315 mg. In embodiments, the therapeutically effective amount to selectively antagonize RAR alpha over RAR beta and RAR gamma in a subject may be about 320 mg. In embodiments, the therapeutically effective amount to selectively antagonize RAR alpha over RAR beta and RAR gamma in a subject may be about 325 mg. In embodiments, the therapeutically effective amount to selectively antagonize RAR alpha over RAR beta and RAR gamma in a subject may be about 330 mg. In embodiments, the therapeutically effective amount to selectively antagonize RAR alpha over RAR beta and RAR gamma in a subject may be about 335 mg. In embodiments, the therapeutically effective amount to selectively antagonize RAR alpha over RAR beta and RAR gamma in a subject may be about 340 mg. In embodiments, the therapeutically effective amount to selectively antagonize RAR alpha over RAR beta and RAR gamma in a subject may be about 345 mg. In embodiments, the therapeutically effective amount to selectively antagonize RAR alpha over RAR beta and RAR gamma in a subject may be about 350 mg. In embodiments, the therapeutically effective amount to selectively antagonize RAR alpha over RAR beta and RAR gamma in a subject may be about 355 mg. In embodiments, the therapeutically effective amount to selectively antagonize RAR alpha over RAR beta and RAR gamma in a subject may be about 360 mg. In embodiments, the therapeutically effective amount to selectively antagonize RAR alpha over RAR beta and RAR gamma in a subject may be about 365 mg. In embodiments, the therapeutically effective amount to selectively antagonize RAR alpha over RAR beta and RAR gamma in a subject may be about 370 mg. In embodiments, the therapeutically effective amount to selectively antagonize RAR alpha over RAR beta and RAR gamma in a subject may be about 375 mg. In embodiments, the therapeutically effective amount to selectively antagonize RAR alpha over RAR beta and RAR gamma in a subject may be about 380 mg. In embodiments, the therapeutically effective amount to selectively antagonize RAR alpha over RAR beta and RAR gamma in a subject may be about 385 mg. In embodiments, the therapeutically effective amount to selectively antagonize RAR alpha over RAR beta and RAR gamma in a subject may be about 390 mg. In embodiments, the therapeutically effective amount to selectively antagonize RAR alpha over RAR beta and RAR gamma in a subject may be about 395 mg. In embodiments, the therapeutically effective amount to selectively antagonize RAR alpha over RAR beta and RAR gamma in a subject may be about 400 mg. In embodiments, the therapeutically effective amount to selectively antagonize RAR alpha over RAR beta and RAR gamma in a subject may be about 405 mg. In embodiments, the therapeutically effective amount to selectively antagonize RAR alpha over RAR beta and RAR gamma in a subject may be about 410 mg. In embodiments, the therapeutically effective amount to selectively antagonize RAR alpha over RAR beta and RAR gamma in a subject may be about 415 mg. In embodiments, the therapeutically effective amount to selectively antagonize RAR alpha over RAR beta and RAR gamma in a subject may be about 420 mg. In embodiments, the therapeutically effective amount to selectively antagonize RAR alpha over RAR beta and RAR gamma in a subject may be about 425 mg. In embodiments, the therapeutically effective amount to selectively antagonize RAR alpha over RAR beta and RAR gamma in a subject may be about 430 mg. In embodiments, the therapeutically effective amount to selectively antagonize RAR alpha over RAR beta and RAR gamma in a subject may be about 435 mg. In embodiments, the therapeutically effective amount to selectively antagonize RAR alpha over RAR beta and RAR gamma in a subject may be about 440 mg. In embodiments, the therapeutically effective amount to selectively antagonize RAR alpha over RAR beta and RAR gamma in a subject may be about 445 mg. In embodiments, the therapeutically effective amount to selectively antagonize RAR alpha over RAR beta and RAR gamma in a subject may be about 450 mg.

In embodiments, the therapeutically effective amount is administered once per day for about 28 days to about 24 weeks, such as about 28 days, about 29 days, about 30 days, about 31 days, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, or any value contained within a range formed by any two of the preceding values.

For example, in embodiments, the therapeutically effective amount is administered once per day for about 13 weeks to about 24 weeks, about 13 weeks to about 20 weeks, about 16 weeks to about 24 weeks, about 20 weeks to about 24 weeks, or any value contained within a range formed by any two of the preceding values. In embodiments, the therapeutically effective amount is administered once per day for about 13 weeks. In embodiments, the therapeutically effective amount is administered once per day for about 14 weeks. In embodiments, the therapeutically effective amount is administered once per day for about 15 weeks. In embodiments, the therapeutically effective amount is administered once per day for about 16 weeks. In embodiments, the therapeutically effective amount is administered once per day for about 17 weeks. In embodiments, the therapeutically effective amount is administered once per day for about 18 weeks. In embodiments, the therapeutically effective amount is administered once per day for about 19 weeks. In embodiments, the therapeutically effective amount is administered once per day for about 20 weeks. In embodiments, the therapeutically effective amount is administered once per day for about 21 weeks. In embodiments, the therapeutically effective amount is administered once per day for about 22 weeks. In embodiments, the therapeutically effective amount is administered once per day for about 23 weeks. In embodiments, the therapeutically effective amount is administered once per day for about 24 weeks.

In embodiments, the therapeutically effective amount is about 15 mg and the therapeutically effective amount is administered once per day for about 13 weeks. In embodiments, the therapeutically effective amount is about 30 mg and the therapeutically effective amount is administered once per day for about 13 weeks. In embodiments, the therapeutically effective amount is about 45 mg and the therapeutically effective amount is administered once per day for about 13 weeks. In embodiments, the therapeutically effective amount is about 90 mg and the therapeutically effective amount is administered once per day for about 13 weeks. In embodiments, the therapeutically effective amount is about 180 mg and the therapeutically effective amount is administered once per day for about 13 weeks. In embodiments, the therapeutically effective amount is about 270 mg and the therapeutically effective amount is administered once per day for about 13 weeks. In embodiments, the therapeutically effective amount is about 300 mg and the therapeutically effective amount is administered once per day for about 13 weeks. In embodiments, the therapeutically effective amount is about 360 mg and the therapeutically effective amount is administered once per day for about 13 weeks. In embodiments, the therapeutically effective amount is about 450 mg and the therapeutically effective amount is administered once per day for about 13 weeks.

In embodiments, the therapeutically effective amount is about 15 mg and the therapeutically effective amount is administered once per day for about 20 weeks. In embodiments, the therapeutically effective amount is about 30 mg and the therapeutically effective amount is administered once per day for about 20 weeks. In embodiments, the therapeutically effective amount is about 45 mg and the therapeutically effective amount is administered once per day for about 20 weeks. In embodiments, the therapeutically effective amount is about 90 mg and the therapeutically effective amount is administered once per day for about 20 weeks. In embodiments, the therapeutically effective amount is about 180 mg and the therapeutically effective amount is administered once per day for about 20 weeks. In embodiments, the therapeutically effective amount is about 270 mg and the therapeutically effective amount is administered once per day for about 20 weeks. In embodiments, the therapeutically effective amount is about 300 mg and the therapeutically effective amount is administered once per day for about 20 weeks. In embodiments, the therapeutically effective amount is about 360 mg and the therapeutically effective amount is administered once per day for about 20 weeks. In embodiments, the therapeutically effective amount is about 450 mg and the therapeutically effective amount is administered once per day for about 20 weeks.

In embodiments, the therapeutically effective amount is about 15 mg and the therapeutically effective amount is administered once per day for about 24 weeks. In embodiments, the therapeutically effective amount is about 30 mg and the therapeutically effective amount is administered once per day for about 24 weeks. In embodiments, the therapeutically effective amount is about 45 mg and the therapeutically effective amount is administered once per day for about 24 weeks. In embodiments, the therapeutically effective amount is about 90 mg and the therapeutically effective amount is administered once per day for about 24 weeks. In embodiments, the therapeutically effective amount is about 180 mg and the therapeutically effective amount is administered once per day for about 24 weeks. In embodiments, the therapeutically effective amount is about 270 mg and the therapeutically effective amount is administered once per day for about 24 weeks. In embodiments, the therapeutically effective amount is about 300 mg and the therapeutically effective amount is administered once per day for about 24 weeks. In embodiments, the therapeutically effective amount is about 360 mg and the therapeutically effective amount is administered once per day for about 24 weeks. In embodiments, the therapeutically effective amount is about 450 mg and the therapeutically effective amount is administered once per day for about 24 weeks.

In embodiments, the therapeutically effective amount may be administered once per day, twice per day, or three times per day. In embodiments, the therapeutically effective amount may be administered twice per day for about 28 days to about 24 weeks. In embodiments, the therapeutically effective amount may be administered three times per day for about 28 days to about 24 weeks.

The compounds of the disclosure may be formulated for administration in any convenient way for use in human or veterinary medicine and the disclosure therefore includes within its scope pharmaceutical compositions comprising a compound of the disclosure adapted for use in human or veterinary medicine. Such compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro edit. 1985). The choice of pharmaceutical carrier can be selected with regard to the intended route of administration and standard pharmaceutical practice. The pharmaceutical compositions may include, in addition to the carrier, any suitable binder(s), lubricant(s), suspending agent(s), coating agent(s), and/or solubilizing agent(s).

The pharmaceutical compositions can be administered orally in the form of tablets containing excipients such as starch or lactose, or in capsules or ovules either alone or in admixture with excipients, or in the form of elixirs, solutions, or suspensions containing flavoring or coloring agents, or they can be injected parenterally, for example intravenously, intramuscularly, or subcutaneously. For buccal or sublingual administration, the compositions may be administered in the form of tablets or lozenges, which can be formulated in a conventional manner.

The pharmaceutical compositions of the present disclosure can be administered in the form of tablets, capsules, troches, ovules, elixirs, solutions, or suspensions, for immediate-, delayed-, modified-, sustained-, pulsed-, or controlled-release applications. The pharmaceutical compositions of the present disclosure may also be presented in the form of solutions, gels, syrups, or suspensions, or a dry powder for reconstitution with water or other suitable vehicle before use. Solid compositions such as tablets, capsules, lozenges, troches, pastilles, pills, boluses, powder, pastes, granules, bullets, or premix preparations may also be used. Solid and liquid compositions for oral use may be prepared according to methods well-known in the art. Such compositions may also contain one or more pharmaceutically acceptable carriers and excipients which may be in solid or liquid form.

In some embodiments, the pharmaceutical composition is a tablet. The pharmaceutical composition may further include a diluent, a disintegrant, a surfactant, a lubricant, a color coating, or any combination thereof.

Oral preparations may optionally include various standard pharmaceutical carriers and excipients, such as binders, fillers, buffers, lubricants, glidants, dyes, disintegrants, odorants, sweeteners, surfactants, mold release agents, antiadhesive agents, and coatings. Some excipients may have multiple roles in the compositions, that is, act as both binders and disintegrants.

The methods and compositions of the present disclosure may be employed in the following embodiments, which may be combined as appropriate to form new embodiments.

### EXAMPLES

The following examples were carried out according to embodiments of the present disclosure.

### Example 1: Phase 1 Study

The compound of the present disclosure, also referred to herein as the compound of formula (I) or Compound A, was evaluated in a Phase 1 study to evaluate toxicity, safety pharmacology, and genotoxicity.

TABLE 1 shows the IND-enabling studies which show the safety of Compound A.

TABLE 1

| GLP study | Study type | Main outcomes |
|---|---|---|
| Rat 28-Day repeat tox | Toxicity | NOAEL 40X over |
| | | therapeutic dose |
| Dog 28-Day repeat tox | | NOAEL 22X over rat |
| | | therapeutic dose |
| hERG | Safety pharmacology | Clean |
| Rat respiratory | | Clean |
| Rat FOB | | Clean |
| Dog cardiovascular | | Clean |
| Rat micronucleus | Genotoxicity | Clean |
| Chromosomal aberration | | Clean |
| Ames | | Clean |

FIG. 8 is a flow chart of the Phase 1 study design, according to an embodiment of the present disclosure.

TABLE 2 shows the clinical laboratory parameters assessed in the study.

TABLE 2

| Hematology | Clinical Chemistry | Virology | Urinalysis | Drugs of Abuse |
|---|---|---|---|---|
| Hemoglobin | Sodium | Hepatitis B surface | Bilirubin | Amphetamines |
| Hematocrit (HCT; packed | Potassium | antigen | Urobilinogen | Barbiturates |
| cell volume [PCV]) | Chloride | Hepatitis C virus | Ketones | Benzodiazepines |
| Red blood cell (RBC; | Bicarbonate | antibody | Glucose | Cocaine |
| erythrocyte) count | Urea | HIV 1 & 2 antibodies | Protein | Marijuana/cannabis |
| Mean corpuscular volume | Creatinine | If Required: | Blood | Methadone |
| (MCV) | eGFR will be calculated at | SARS-CoV-2 antigen | Nitrites | Methamphetamine/ |
| Mean corpuscular | screening using the CKD- | Pharmacodynamics | pH | ecstasy |
| hemoglobin (MCH) | EPI (2009) equation for | Follicle | Specific gravity | Morphine/opiates |
| Mean corpuscular | eligibility purposes | Stimulating | Leukocytes | Phencyclidine |
| hemoglobin | Bilirubin (total) | Hormone | At discretion of | Tricyclic |
| concentration | Bilirubin (direct; only if | Luteinizing hormone | investigator based on | antidepressants |
| (MCHC) | total is elevated) | Testosterone | urinalysis results | |
| Platelet count | Alkaline phosphatase | Sex hormone binding | Microbiology | |
| White blood cell (WBC; | Aspartate aminotransferase | globulin | Urine microscopy | |
| leukocytes) count | (AST) | | | |
| Neutrophils | Alanine aminotransferase | | | |
| Lymphocytes | (ALT) | | | |
| Monocytes | Lactate dehydrogenase | | | |
| Eosinophils | (LDH) | | | |
| Basophils | Creatine kinase (CK) | | | |
| Coagulation Tests | Gamma glutamyl transferase | | | |
| Prothrombin time | (GGT) | | | |
| Activated partial | Protein (Total) | | | |
| thromboplastin time (APTT) | Albumin | | | |
| Fibrinogen | Calcium | | | |
| | Glucose (fasting) | | | |
| | Glucose | | | |
| | Triglycerides (fasting) | | | |
| | Cholesterol (total; fasting) | | | |

TABLE 3 shows the pharmacokinetic parameters of Compound A at various doses. Without wishing to be bound by theory, it is contemplated that a low dose of Compound A may be efficacious in part due to its long half-life.

TABLE 3

| | | Tₘₐₓ | Cₘₐₓ | AUC₀₋₂₄ | AUC₀₋ₗₐₛₜ | AUC_{0-inf} | T_{1/2} |
|---|---|---|---|---|---|---|---|
| | | (h) | (ng/mL) | (h*ng/mL) | (h*ng/mL) | (h*ng/mL) | (h) |
| | | Geometric Mean (CV %) | | | | | |
| 10 | mg | 8.00* | 131 | 2,380 | 8,180 | 9,560 | 53.5 |
| | | (4.00-8.00) | (41.7) | (47.8) | (45.2) | (43.6) | (36.5) |
| 30 | mg | 4.00* | 200 | 3,520 | 12,400 | 12,900 | 61.7 |
| | | (3.00-12.0) | (77.8) | (75.8) | (67.9) | (65.9) | (29.6) |
| 90 | mg | 8.00* | 472 | 8,430 | 26,300 | 26,800 | 71.3 |
| | | (4.00-8.00) | (64.9) | (67.9) | (53.1) | (52.4) | (20.8) |
| 180 | mg | 8.00* | 1,750 | 27,300 | 73,400 | 75,400 | 76.3 |

| | | | | | |
|---|---|---|---|---|---|
| (4.00-8.00) | (31.5) | (26.3) | (21.1) | (21.4) | (32.9) |

*Shown is median (range)

### Non-Human Primate PK and Efficacy Studies

Animals and husbandry. Six (6) sexually mature, non-naïve male Cynomolgus macaques (8-15 years, 6-15 kg) were used. The animals were individually housed in a humidity- and temperature-controlled facility under a 12 h light/12 h dark diurnal cycle with two daily meals and water ad libitum. The animals were provided with cage toys and daily fresh fruits for environmental enrichment. The animals were acclimated for 2 weeks and observed twice daily to assess overall health and behavior. The animal use protocol was approved by WuXi App Tec's IACUC per the Association for Assessment and Accreditation of Laboratory Animal Care (AAALAC) guideline. The conduct of the study followed the Guidance for the Care and Use of Laboratory Animals (NRC 2011, 8th Edition), with a USDA Category C for the pain and distress level. The humane endpoints included: significant body weight drop of 20% within 7 days, severe adverse effort associated with the administration of the test article, pain and distress caused by any of the in-life procedures, and injury sustained during any time of the experiment that could impact the well-being of the animal (all determined by attending veterinarian). All animals were non-naïve and only animals that had normal hematology and serum chemistry parameters, and normal baseline sperm counts were included in the studies. After the end of the study, all animals were returned to the colony. Confounders were not controlled.

Dosing regimens. Compound A was suspended in 0.9% saline to prepare working solutions of desired concentrations, which were administered to the animals via oral gavage at a dosing volume of 5 mL/kg. The formulation (Compound A in saline) was prepared freshly each day of dosing. Single dose administration: Three (3) animals received a single dose of 1, 5, and 10 mg/kg of Compound A with a 7-day washout period between doses. Repeat dose administration: Three (3) Cohort 1 animals were dosed at 0.5 mg/kg/day (Day 1-46) followed by 1.5 mg/kg/day (Day 47-51), 2.5 mg/kg/day (Day 52-101) and 5 mg/kg/day (Day 102-108). Three (3) Cohort 2 animals were dosed at 5 mg/kg/day (Day 1-30) followed by 7.5 mg/kg/day (Day 31-37). No control animals were used in the single and repeat dose studies. Since bioavailability was assessed at time point zero in the PK study and sperm parameters were assessed at pre-dose in the efficacy studies, each animal served as its own control. Animals were not randomized as all animals per group received the same dose levels of Compound A. No formal sample size calculations were performed as 3 animals per group were considered a sufficient minimum number of animals for this exploratory pilot study to assess bioavailability and efficacy.

PK assessments. In the single dose study and on Days 1 and 28 of the repeat dose studies, blood samples were drawn from all 3 animals at t=0 (pre-dose) and 0.5, 1, 2, 4, 8, 24, 48, 72, 96, 120, 144 and 168 h post-dose to assess plasma concentrations of Compound A over time. Blood samples were collected at single time points on the last day of dosing and at the end of the recovery period in the repeat dose studies. At each time point, approximately 1 mL of whole blood was collected into an EDTA-K2 anticoagulant tube from a vein of the left forelimb for PK analysis. Plasma was separated by centrifugation at 3500×g for 10 min at 4° C. and stored frozen at −80° C. until LC/MS/MS analysis. No data point was excluded from the analysis. Pharmacokinetic analyses of plasma Compound A concentration-time data were conducted using non-compartmental Model 200 of WinNonlin®, version 5.2 (Pharsight Corporation, St Louis, MO).

LC/MS/MS analysis. LC/MS/MS analysis was performed using a LCMS-8060 triple quadrupole mass spectrometer (Shimadzu, Kyoto, Japan) coupled with a Triple Quad 5500+ mass spectrometer (Sciex, Framingham, MA). LC was performed using an InertSustain C18 HP column (50× 2.1 mm, 3 μm; GL Sciences Inc., Tokyo, Japan). Elution was isocratic using H20/0.1% formic acid and 100% acetonitrile/0.1% formic acid flowing at 0.4 mL/min. The total run time was 4.0 min. Compound A eluted at 1.97 min, and glibenclamide (internal standard) at 1.35 min. MS/MS was performed with the instrument operating in electrospray positive ion mode (ES+).

Semen collection and sperm count/concentration assessment. Semen samples were obtained once per week or once every other week via electroejaculation during the entire repeat dose study periods from all 3 animals per Cohort to assess sperm concentration and count. Samples were left at room temperature for 30 min to allow for liquefication. Liquefied semen was diluted with 3% NaCl solution for manual sperm count assessment using a hemocytometer. All samples were analyzed in triplicates. Sperm concentration per ejaculate was calculated with the following formula: Sperm concentration=Sperm count×104×Dilution factor. No data point was excluded from the analysis.

Testicular biopsy collection and histology. Testicular biopsies (2 per animal; one from the right and one from the left testicle) were taken 24 h after administering the last dose and on the last day of the recovery period from all 3 animals per Cohort to assess morphological changes. Preoperative care of animals consisted of food restriction and general examination. All surgical procedures were conducted using aseptic techniques and comprehensive physiologic monitoring. The animals were anesthetized with Zoletil 50 (5 mg/kg) by intramuscular (i.m.) injection. The scrotum was disinfected with iodine and 75% alcohol, and lidocaine (1%) was administered intradermally at the biopsy needle insertion site. Core biopsies (one per testis) were collected with a 16 G TSK starcut biopsy needle. Immediately after biopsy collection, animals received Meloxicam (0.2 mg/kg) and Ketorolac (10 mg) for pain relief, and Ceftriaxone sodium (0.5 g) as an anti-inflammatory (all via i.m. injection). Meloxicam was given once daily for 3 consecutive days, Ketorolac twice daily for 2 days, and Ceftriaxone sodium once daily for 4 days post-collection. Testicular tissue was transferred to Neutral Buffered Formalin (NBF) for subsequent histological preparation. Fixed tissue was embedded in paraffin, and morphological examination was performed with H&E-stained slides.

Clinical pathology and hormone levels. Blood samples were collected at pre-dose, on Day 29, 24 h after administering the last dose, and on the last day of the recovery period from all 3 animals per Cohort to assess hematology and serum chemistry parameters, and hormone levels. Whole blood was collected into EDTA-K2 anticoagulant tubes for complete blood count (CBC) analysis. Whole blood was collected into serum separator tubes for a comprehensive metabolic panel, lipid panel, testosterone, FSH, and inhibin B levels. The serum was separated by centrifugation at 3500×g for 10 min at 4° C. and stored frozen at −80° C. until analysis. Testosterone, FSH, and inhibin-B levels in serum were analyzed with ELISA. Each sample per animal and time point was measured in duplicates, and the average was calculated. No data point was excluded from the analysis.

### Statistics and Reproducibility

Three independent measurements were performed for the transactivation assays. All other measurements were taken from distinct samples, and no sample was measured repeatedly.

NHP experiments: One-way ANOVA statistical analyses were performed for hematology parameters, serum chemistry parameters, and hormone levels to assess changes from baseline with Origin 2022 version 9.9.0.225. Per time point, samples from 3 animals were analyzed.

### Example 2: Phase 1B/2A Study

Open label dose escalation study to evaluate safety, tolerability, pharmacokinetics, and pharmacodynamics of repeated daily oral dosing of Compound A for 28 days in healthy men

Indication: Reduction of sperm count and sperm motility as a means for male contraception

### Objective Endpoints:

### Primary:

The primary objective is to assess the safety and tolerability of repeated daily oral dosing of Compound A for 28 days.

The primary endpoint is the incidence of Adverse Events (AEs), abnormal laboratory test results (clinical chemistry and liver function tests, CBC with differential, and lipid panel), ECGs (12-lead and Holter monitoring), vital signs, and other safety assessments.

### Secondary:

The secondary objectives are to evaluate the pharmacokinetics (PK) and pharmacodynamics (PD) of Compound A.

The secondary endpoints are:

Pharmacodynamic semen parameters of Compound A, including ejaculate volume, ejaculate pH, total sperm count per ejaculate, sperm concentration, total motility [progressive, non-progressive, immotile], and sperm morphology.

Plasma PK parameters after 28 days of dosing: area under the curve from time 0 extrapolated to infinity [AUCinf], area under the curve from time 0 to the last measured concentration [AUC0-t], area un-der the curve from time 0 to 24 hours [AUC0-24], time to maximum concentration [Tₘₐₓ], terminal elimination half-life, [T1/2], lag time [T_{lag}], apparent volume of distribution [Vz/F], oral clearance [CL/F], and maximum concentration [Cₘₐₓ] of Compound A.

Compound A concentrations in semen.

Pharmacodynamic parameters of Compound A, including serum hormones (follicle-stimulating hormone [FSH], luteinizing hormone [LH], testosterone [T]), and sex hormone binding globulin (SHBG).

### Exploratory:

The exploratory objectives are:

To evaluate the effect of Compound A on change from baseline in corrected QT interval in ECG (QTc) following 28 oral doses in healthy male participants.

To evaluate the effect of 28-Day dosing with Compound A on in-flammatory biomarkers.

The exploratory endpoints are:

Formal statistical analysis of relationship between the change from baseline in QT interval corrected for heart rate using Bazett's and Fridericia's formulation (AQTcF) and plasma Compound A con-centration.

Formal statistical analysis of AQTcF at each post-dose timepoint for the comparison between each active dose level and placebo.

Inflammatory biomarker parameters: TNF-alpha, IL-6, E-selectin, vWF and hsCRP.

Rationale: Gene targeting research has demonstrated that loss of function of retinoic acid receptors (RARs) inhibits spermatogenesis. Compound A is an orally administered RAR-alpha antagonist that reversibly inhibits spermatogenesis, spermiation, and sperm motility in animal models. The purpose of this 28-Day repeat-dose escalating study is to evaluate the safety, tolerability, PK, and PD of Compound A in healthy human adults. Data from this study will inform future program development.

Study Design: This is a Phase 1b/2a, multicenter, open label, 28-Day repeat-dose escalation study of Compound A in healthy men to evaluate the safety, tolerability, PK and PD, and to assess sexual function and mood. Cohorts will be dosed in the fasted or fed state. In the Phase 1b part, 4 dosing cohorts and one optional 5th cohort with 4 participants each will be evaluated. All participants will receive Compound A. Participants may be replaced automatically if prior to any dosing. If a participant is required to be replaced post-dose, the replacement may be approved at discretion of Sponsor and PI to ensure available data in at least 3 evaluable participants per cohort. In the Phase 2a part, up to 3 cohorts will receive doses within the range of doses that was well tolerated and had biological activity in the Phase 1b portion. Each Phase 2a cohort will have up to 10 participants. All participants will receive Compound A. Participants who discontinue early may be replaced.

All participants will undergo preliminary screening procedures for the study (Day −28 to Day −1). Following successful enrollment into the Phase 1b part of the study, participants will be admitted in the morning of Day −1 (day before dosing), will receive study drug (Compound A) on Days 1-28, and will remain onsite until Day 7 (day of discharge). Participants will return to the clinic on Days 10, 14, 17, 21 and 24 for self-dosing under the supervision of clinic staff and have safety, PK, and PD assessments performed. Participants will be readmitted to the clinic prior to dosing in the morning on Day 27 (the day before the last dosing) and remain onsite until 72 h post last dose (Day 31). On Days 1-7, participants will receive study drug (Compound A) under the supervision of clinic staff. On Days 7, 14 and 21, participants will receive a 7-Day supply of study drug (9 doses in total [7 regular, 2 extra]) for self-dosing. On Days 8, 9, 11, 12, 13, 15, 16, 18, 19, 20, 22, 23, 25 and 26, participants will self-dose at home under the supervision of a telehealth staff member via video call. On Days 10, 14, 17, 21 and 24, participants will return to the clinic for self-dosing under the supervision of clinic staff and have safety, PK, and PD assessments performed.

Following successful enrollment into the Phase 2a part of the study, participants will be admitted in the morning of Day −1 (day before dosing) and will remain onsite until Day 3 (day of discharge). Participants will be readmitted to the clinic prior to dosing in the morning on Day 27 (the day before the last dosing) and remain onsite until 72 h post last dose (Day 31). On Days 1-3, participants will receive study drug (Compound A) under the supervision of clinic staff. On Day 3, participants will receive a 4-Day supply of study drug (6 doses in total [4 regular, 2 extra]), and on Days 7, 14 and 21, participants will receive a 7-Day supply of study drug (9 doses in total [7 regular, 2 extra]) for self-dosing. On Days 4, 7, 10, 14, 17, 21 and 24, participants will return to the clinic for self-dosing under the supervision of clinic staff and have safety, PK and PD assessments performed. On Days 5, 6, 8, 9, 11, 12, 13, 15, 16, 18, 19, 20, 23, 25 and 26, participants will self-dose at home under the supervision of a telehealth staff member via video call.

Follow-up telehealth visits (phone call) will be performed on Days 35, 42 and 49 (+/−1 day) to ensure the ongoing wellbeing of all Phase 1b and Phase 2a participants. In-person follow-up visits will be performed with all participants on Day 42 (+/−1 day), and on Days 56, 84, 112, 140, 168, and 196 (+/−2 days) to assess PK and/or sperm parameters. Day 42 is the only day when participants will have a telehealth visit and an in-person visit to the fertility clinic for semen/sperm parameter assessment. If sperm parameters have not returned to at least the 5th percentile of the WHO range of normality (WHO, 2010) by Day 196 (earliest End of Study visit; approximately 2.5 spermatogenic cycles), sperm parameters will also be assessed until the resolution of any clinically relevant changes in their sperm parameters (as assessed at visits on Days 224, 252, and 280+/−2 days). The last possible End of Study visit will be Day 280+/−2 days.

After at least 3 evaluable participants in a cohort of the Phase 1b part complete the 28-day dosing, safety data and available PK and PD data from up to Day 28 will be reviewed to determine the dose level for the subsequent cohort.

After the review of the safety, and available PK and PD data from up to Day 28 of at least 3 evaluable participants of the last cohort of the Phase 1b part, the Phase 2a part of this study will be initiated. The doses for the Phase 2a cohorts will be selected based on emerging data from the Phase 1b part.

Planned number of participants: In the Phase 1b part, 4 dosing cohorts and one optional 5th cohort with 4 participants will be evaluated (up to 20 participants). Participants may be replaced automatically if prior to any dosing. If a participant is required to be replaced post-dose, the replacement may be approved at discretion of Sponsor and PI to ensure available data in at least 3 evaluable participants per cohort. In the Phase 2a part, up to 3 cohorts of up to 10 participants (up to 30 participants) are planned. This number may increase as participants may be replaced. All participants will receive Compound A. Any replacement participants will receive the same dose as the participant they are replacing.

An evaluable participant is a study participant who has completed the planned safety assessments up to Day 28, and planned PK assessments up to 24 h after dosing on Day 1 and Day 28. However, participants withdrawn due to an investigational medicinal product (IMP)-related AE will be considered as an evaluable participants and not be replaced.

Test product: Compound A Capsule, 15, 45, 90 and 180 mg. Dose, route, frequency: Once daily single oral dose for 28 days. For the Phase 1b part, planned doses are 15, 45, 90 and 180 mg. For the Phase 2a part, doses will be determined based on the Phase 1b results, but the highest dose will not exceed the highest well tolerated dose from the Phase 1b part.

Treatment Regimes: All participants will fast (except water) for at least 10 hours overnight prior to administration of study drug (Compound A), and for at least 2 hours after dosing. Study drug (Compound A) will be administered with 240 mL of water in the clinic by clinic staff and at home by self-dosing under supervision of telehealth staff via video call. Water will be allowed ad libitum during the study, except for the time period between 1 hour before dosing and 1 hour after dosing, when the only water permitted will be the water administered with the study drug. Decaffeinated fluids will be allowed ad libitum from no earlier than 2 h post-dose. A late breakfast or morning snack should be taken no earlier than 2 h post-dose. Lunch should be taken at approximately 4 h post-dose, an evening meal at approximately 10 h post-dose, and an evening snack should be finished no later than 12 h post-dose.

The starting dose for this study is 15 mg, with planned escalation to 45, 90 and 180 mg. In the Phase 1b part, the dose escalation review committee (DERC) will utilize safety, tolerability and available PK and PD data to determine the dose level for the subsequent cohort. In the Phase 2a part, the DERC will select dose levels based on safety, tolerability and available PK and PD data from the Phase 1b part and any previous Phase 2a cohort(s). In order to maximize the dose response information gained from the study, in either part, the next cohort may be given a dose that is lower than planned or that is between two prior doses or a different regimen.

### Criteria for Evaluation:

Safety Parameters: Each participant's health status will be monitored carefully throughout the study. Baseline data collected at the screening visit will consist of the medical and andrological history, detailed physical examination (including height, weight, and BMI), ECG (12-lead and Holter monitoring), vital signs, laboratory tests (including clinical chemistry, liver function, CBC with differential, hormones [serum T, FSH, LH, E2, and calculated free T], SHBG, HIV/hepatitis screening), urinalysis including toxicology, and semen analysis including ejaculate volume, ejaculate pH, total sperm count per ejaculate, sperm concentration, total motility (progressive, non-progressive, immotile), and sperm morphology.

Specific assessments to evaluate treatment safety include the following: the frequency and type of adverse events; safety laboratory tests (clinical chemistry and liver function tests), CBC with differential, and lipid panel; ECGs (12-lead and Holter monitoring); and vital signs.

Changes in blood counts and clinical chemistry measures from baseline to recovery will be calculated for all participants in the treated population with available data. All of these data will be compared to mean changes from baseline and used to identify the incidence of clinically significant results indicative of safety concerns.

### Pharmacokinetics:

Participants will have overnight stays in the clinical research unit in order to monitor their wellbeing and obtain 24-hour PK profiles on Day 1 and Day 28. For PK profiles, blood samples will be collected at −0.5 hours before (Day 1 only), and 0.5, 1, 2, 4, 6, 8, 12, 18 and 24 hours after oral dose administration.

Single time point blood samples will be collected prior to dosing on Days 3, 4, 7, 10, 14, 17, 21 and 24. Elimination will be assessed by measuring serum concentrations of Compound A at 48 and 72 hours (Days 30 and 31) and 28 days (Day 56) after the last dose.

Compound A concentrations in semen will be assessed once during screening, and on Days 21, 28 and 56. If Compound A is detectable in semen on Day 56, semen PK will also be analyzed on Day 84.

### Pharmacodynamics:

Hormones (serum T, LH, FSH, E2, and calculated free T), SHBG and inflammatory biomarkers (TNF-alpha, IL-6, E-selectin, vWF and hsCRP) will be measured once during screening, at −0.5 hours pre-dose (Day 1), on Days 3, 7, 14, 21, 28, 56 and 196 (earliest End of Study Visit); if necessary also on Days 224 or 252 (possible End of Study visits) or on Day 280 (latest End of Study visit).

Statistical methods: The following analysis populations are planned for this study:

Screening Population: all participants who provide informed consent and demographic and/or baseline Screening assessments.

Safety Population: all participants who receive any amount of study drug.

Per Protocol Population: all participants who receive at least 80% of the planned doses of study drug.

Pharmacokinetic Population: all participants who receive study drug and have at least 7 PK samples collected on Days 1 and 28.

Pharmacodynamic Population: all participants who receive study drug and have at least 2 evaluable semen samples collected starting on Day 21 or later during the study.

### Participant Characteristics and Disposition:

The number of participants completing the study, and withdrawing from the study, along with reasons for withdrawal, will be tabulated overall and by treatment group. The number of participants in each analysis population will be reported. Demographic data will be summarized and include age, height, weight, race, ethnicity, prior and concomitant medications, and medical and andrological history.

### Safety Analyses:

Safety data, including AEs, safety laboratory tests (clinical chemistry and liver function tests, CBC with differential, and lipid panel), vital signs, ECGs, and physical examinations, will be summarized. Changes in blood counts, coagulation, and clinical chemistry measures from baseline to recovery will be calculated for all participants.

### Clinical Pharmacology Analyses:

Non-compartmental methods will be used to calculate PK parameters for Compound A where appropriate. PK profiles and parameters collected over 24 hours on Days 1 and 28 will be evaluated and summarized.

Dose proportionality will be evaluated using a power model for log transformed PK parameters Cₘₐₓ, AUC₀₋ₜ, and AUC_{inf} including criteria defined by Hummel et al. (Hummel et al., 2009).

Pharmacodynamic variables will be summarized using descriptive statistics including changes from baseline.

Sample size determination: No formal sample size calculations were performed. The cohort size was estimated based on Shen et al. (Shen et al., 2019). For the Phase 1b part, it is planned to enroll 16 healthy male participants in total (4 cohorts of 4 participants) or 20 participants if the optional 5th cohort is recruited. As many participants per cohort may be replaced as needed to ensure a minimum of 3 evaluable participants per Phase 1b cohort. For the Phase 2a part, it is planned to enroll up to 30 healthy male participants in total (3 cohorts of up to 10 participants); participants who discontinue from the Phase 2a part may or may not be replaced.

Study and treatment duration: The participants for the 28-day repeat-dose study will be evaluated for approximately 32 to 44 weeks (screening up to 4 weeks, dosing 4 weeks, and post-dosing between 24 and 36 weeks).

### LIST OF ABBREVIATIONS

| Abbreviation | Definition |
|---|---|
| ADR | Adverse drug reaction |
| AE | Adverse event |
| AESI | Adverse event of special interest |
| ALT | Alanine aminotransferase |
| AST | Aspartate aminotransferase |
| AUC | Area under the curve |
| AUC₀₋₂₄ | Area under the curve from time 0 to 24 hours |
| AUC₀₋ₜ | Area under the curve from time 0 to the last measured |
| | concentration |
| AUC_{inf} | Area under the curve from time 0 extrapolated to infinity |
| BMI | Body mass index |
| BP | Blood pressure |
| BSA | Body surface area |
| Cₘₐₓ | Maximum concentration |
| CRA | Clinical Research Associate |
| CSR | Clinical Study Report |
| CTCAE | Common Terminology Criteria for Adverse Events |
| DERC | Dose Escalation Review Committee |
| DILI | Drug-induced liver injury |
| E2 | Estradiol |
| ECG | Electrocardiogram |
| eCRF | Electronic case report form |
| FDA | Food and Drug Administration |
| FSH | Follicle-stimulating hormone |
| GCP | Good Clinical Practice |
| GLP | Good Laboratory Practice |
| HCT | Hematocrit |
| HED | Human equivalent dose |
| HGB | Hemoglobin |
| HIV | Human immunodeficiency virus |
| hs-cTnT | Highly sensitive cardiac troponin test |
| IB | Investigator's Brochure |
| ICF | Informed consent form |
| ICH | International Council for Harmonisation |
| IEC | Independent Ethics Committee |
| IL | Interleukin |
| GCP | Good Clinical Practice |
| LH | Luteinizing hormone |
| MAD | Multiple ascending doses |
| Medsafe | New Zealand Medicines and Medical Devices Safety |
| | Authority |
| NHP | Nonhuman primate(s) |
| NOAEL | No-observed-adverse-effect level |
| PD | Pharmacodynamic(s) |
| PK | Pharmacokinetic(s) |
| QTc | Duration of corrected QT interval in ECG |
| RAR | Retinoic acid receptor |
| RBC | Red blood cell |
| SAD | Single ascending doses |
| SAE | Serious adverse event |
| SAP | Statistical Analysis Plan |
| SARM | Selective Androgen Receptor Modulator |
| SD | Standard deviation |
| SHBG | Sex hormone binding globulin |
| SoA | Schedule of Assessments |
| SSRI | Selective Serotonin Reuptake Inhibitor |
| T | Testosterone |
| T_{1/2} | Half-life (lives) |
| TGA | Therapeutic Goods Administration |
| TNF | Tumor necrosis factor |
| ULN | Upper limit of normal |

### 3.1: Nonclinical Experience

### 3.1.1: Efficacy

No efficacy was observed after single dose studies in the rat. Single doses as high as 1,000 mg/kg in rats had no impact on sperm parameters on Day 15 post dose (the last day of evaluation) (see Section 3.1.3).

Repeat dose studies in mice, rats and NHPs demonstrated decreases in sperm counts at Com-pound A doses as low as 2.5 mg/kg/day for 4 weeks (mouse), 0.75 mg/kg/day for 36 days (rat) and 2.5 mg/kg/day for 7 weeks (NHP), as well as the reversibility of this antispermatogenic effect within 1 spermatogenic cycle in mice and 1.5-3.5 spermatogenic cycles in NHPs. Rat testicular toxicity reversibility characteristics and findings after repeat dosing are discussed in Section 3.1.4.

Mating trials with mice showed 99% infertility (based on the number of viable embryos) when dosed at 10 mg/kg/day for 4 weeks and complete recovery of fertility within 6-10 weeks after cessation of dosing. Mating trials with rats showed that fertility was reduced to 13%, 6% and 0% in the 3, 10 and 30 mg/kg/day dose groups, respectively, compared to 88% in the control. Dog histopathology confirmed intended testicular toxicity (functional impairment and expected morphological changes) after 28-day dosing at doses ≥2.5 mg/kg/day and recovery of spermatogenesis during 1 spermatogenic cycle.

### 3.1.2: Safety Pharmacology and Genotoxicity

A standard battery of nonclinical good laboratory practice (GLP) safety studies, including safety pharmacology, genotoxicity, single dose and repeat-dose toxicity studies have been conducted. In safety pharmacology studies, there were no adverse effects on central nervous system (CNS) or respiratory system in male Sprague Dawley rats following single oral doses of 3, 10 or 30 mg/kg and the no-observed-adverse-effect level (NOAEL) was 30 mg/kg in both the CNS and respiratory studies. In male Beagle dogs, there were no effects in the single dose cardiovascular study at doses of 2.5, 5 or 25 mg/kg and the NOAEL was 25 mg/kg.

Compound A was not genotoxic in the in vitro bacterial or mammalian cell assays or the in vivo rat micronucleus study at the highest doses tested.

In a rat single dose study, the NOAEL was 1,000 mg/kg. In 28-day toxicity studies, the respective NOAEL in male Sprague Dawley rats and male Beagle dogs were 30 mg/kg and 5 mg/kg. No signs of toxicity were noted at dose levels equal to or below the NOAEL during the respective dosing periods, apart from intended testicular toxicity (functional impairment and expected morphological changes).

### 3.1.3: Acute Toxicity

Single dose studies with Compound A demonstrated that male rats, mice, and dogs tolerated 1,000 mg/kg (161.3 mg/kg HED; GLP study), 1,000 mg/kg (81.3 mg/kg HED; non-GLP study), and 500 mg/kg (277.8 mg/kg HED; non-GLP study), respectively, as the highest doses tested.

### 3.1.4: Repeat Dose Toxicity

A GLP study with male rats dosed at 3-30 mg/kg/day for 28 days demonstrated a NOAEL of 30 mg/kg/day (4.8 mg/kg/day HED), which represents a 40-fold increase over the minimum efficacious dose in rats, 0.75 mg/kg/day (based on exposure). In all dose groups, rats showed no signs of toxicity apart from the intended testicular toxicity (functional impairment and expected morphological changes). The rat recovery is very slow and may not be achieved after the doses given in this study (3, 10 or 30 mg/kg/day for 28 days) because these doses resulted in severe testicular effects, thus there is not enough critical mass present to initiate spermatogenesis. In addition, testicular histopathology looked very much identical at all dose levels tested. This lack of a dose response indicates that testicular insult at all dose levels tested was too severe to allow for reversibility. The ability to recover, and the process of recovery, from testicular injury and disruption in spermatogenesis appears to be unique in rats. Rats have been shown to have an androgen-mediated block to stem cell spermatogonial proliferation. The requirement of a change in intratesticular testosterone to resume spermatogenesis post testicular insult is unique to rats and has no translation into humans. Rat prostate weight (which is androgen-sensitive) was unchanged after dosing at 3, 10 or 30 mg/kg/day for 28 days indicating that Compound A did not change testosterone levels during the study. It is therefore likely that intratesticular testosterone levels remained unchanged, thus maintaining the androgen-mediated block to spermatogenesis post-testicular insult.

A GLP study with male dogs dosed at 2.5-25 mg/kg/day for 28 days demonstrated a NOAEL of 5 mg/kg/day (2.8 mg/kg/day HED), which represents a 22-fold increase over the minimum efficacious dose in rats. The dog was the more sensitive toxicology species, and dogs showed no signs of toxicity at dose levels within the NOAEL apart from the intended testicular toxicity. Spermatogenesis was reversible over a 62-day recovery period. Major safety findings in dogs at dose levels above the NOAEL (10 and 25 mg/kg/day [5.6 and 13.9 mg/kg/day HED]) included deaths and moribundity during the first week of the study; the remaining animals in these groups were removed from the study. The cause of death or moribundity in dogs given 10 or 25 mg/kg/day was attributed to renal toxicity, although other findings (cardiovascular changes) potentially secondary to renal toxicity were also observed.

An exploratory 14-day non-GLP study was conducted in male NHPs (cynomolgus monkeys) for assessment of efficacy and toxicity parameters prior to the conduct of the 28-day GLP toxicity studies in rats and dogs. The NHPs were dosed at 5-20 mg/kg/day for 14 days and demonstrated a NOAEL of 10 mg/kg/day (3.2 mg/kg/day HED), which represents a 22-fold increase over the minimum efficacious dose in rats. No signs of toxicity were noted at dose levels within the NOAEL. Major safety findings in NHPs at dose levels above the NOAEL (20 mg/kg/day [6.5 mg/kg/day HED]) included elevated liver enzymes, with all animals surviving.

### 3.1.5: Pharmacokinetics

Pharmacokinetics (PK) data for Compound A in rat (GLP single dose and 28-day repeat-dose studies), dog (GLP 28-day repeat-dose) and NHP (non-GLP 14-day repeat-dose) at the NOAEL are summarized in TABLE 4.

TABLE 4
Summary of PK Data in Rats, Dogs, and Monkeys at the NOAEL

| | | | Human | | | | | | |
|---|---|---|---|---|---|---|---|---|---|
| | | | dose 60 kg, | | | Cₘₐₓ | Tₘₐₓ | T_{1/2} | AUC₀₋₂₄ |
| | NOAEL | HED | safety | | | (ng/mL) | (h) | (h) | (h*ng/mL) |
| Species | (mg/kg) | (mg/kg) | factor 10 | | Day | Mean ± SD | | | |
| Rat, single | 1,000 ^{d} | 161.29 | 967.74 | mg | 1 | 45,100 ± | 48.0 | 16.9 | 560,000 ± |
| dose, n = 20 | | | | | | 26,600 | (8.0-96.0) ^{a} | (8.4-25.4) ^{a, b} | 200,000 |
| Rat, 28-day | 30 ^{d} | 4.84 | 29.04 | mg | 1 | 9,310 ^{c} | 8.0 ^{c} | ND | 131,000 ^{c} |
| repeat-dose; | | | | | 28 | 6,770 ^{c} | 4.0 ^{c} | ND | 86,400 ^{c} |
| n = 3 | | | | | | | | | |
| Dog, 28-day | 5 | 2.78 | 16.68 | mg | 1 | 3,410 ± | 4.0 | 13.9 | 43,800 ± |
| repeat-dose; | | | | | | 1,040 | (2.0-12.0) ^{a} | (6.5-24.8) ^{a} | 12,200 |
| n = 9 | | | | | 28 | 4,800 ± | 2.0 | 18.7 | 80,800 ± |
| | | | | | | 1,330 | (2.0-12.0) ^{a} | (11.8-72.6) ^{a} | 19,700 |
| NHP, 14-day | 10 | 3.23 | 19.38 | mg | 1 | 13,033 ± | 2.7 ± 1.2 | 7.9 ± 1.7 | 146,744 ± |

| | | | | | |
|---|---|---|---|---|---|
| repeat-dose; | | 1,415 | | | 29,478 |
| n = 3 | 14 | 6,533 ± | 4.0 ± 0.0 | 6.2 ± 0.4 | 69,704 ± |
| | | 1,698 | | | 19,075 |

^{a} Data is presented as median (range).
^{b} Calculated from only 2 animals.
^{c} SD was not calculated because blood was taken from 3 animals/group/time point as per alternating sampling design.
^{d} Highest dose tested
Abbreviations:
AUC₀₋₂₄ = area under the curve from time 0 to 24 hours;
Cₘₐₓ = maximum concentration;
HED = human equivalent dose;
ND = not determined due to less than 3 quantifiable time points after Tₘₐₓ;
NHP = nonhuman primate;
NOAEL = no-observed-adverse-effect level;
PK = pharmacokinetic(s);
T_{1/2} = half-life;
Tₘₐₓ = time to maximum concentration

### 3.1.6: Metabolism and Excretion

The stability of Compound A was tested in vitro in microsomes of different species. After 90 minutes in human liver microsomes, 92% of Compound A remained intact, compared to 100% in monkeys, 110% in mice, 113% in rats, 99% in rabbits, and 101% in dogs. Respective half-lives (T1/2) were 9.5 hours (human), 23.1 hours (monkeys), >23.1 hours (mice), >23.1 hours (rats), 19.6 hours (rabbits), and >23.1 hours (dogs).

Metabolite Identification of Compound A was evaluated in vitro using pooled hepatocytes from human (10 males), monkey (male cynomolgus macaque), mouse (male CD-1), rat (male Sprague-Dawley), rabbit (male New Zealand) and dog (male Beagle). Biotransformation of Compound A at a concentration of 10 μM yielded 10 metabolites. Overall, each metabolite was present at less than 10% of the recoverable metabolic profile across all species. The rat produced 6 of the 8 quantified human metabolites and the dog produced 4 of the 8 human metabolites. An unknown metabolite representing 1.59% of recovered metabolites and parent drug and a glutathione conjugate representing 0.25% of recovered metabolites and parent drug in human hepatocytes, were not identified in either the rat or dog metabolite profiles. Because the presence of these metabolites was ≤10% of the recoverable metabolic profile, and because all other human metabolites were represented in either the rat or dog, these 2 species were considered relevant species to assess toxicity.

Binding of Compound A to plasma proteins was evaluated in vitro using pooled male and female human, monkey (cynomolgus macaque), mouse (CD-1), rat (Sprague-Dawley), and dog (Beagle) blood. Binding of 1 μM Compound A was at least 99.99% in all species tested.

### 3.2: Clinical Experience

The safety, tolerability, PK, and PD of single ascending oral doses of Compound A were studied in a double-blind, placebo controlled, first in human Phase 1a study with healthy vasectomized men. The doses tested under fasting conditions were 10, 30, 90 and 180 mg. A total of 16 participants in 2 cohorts of 8 participants were enrolled to receive two doses of study drug (Compound A or matching placebo). Cohort 1 first received 10 mg, and after a 5-week washout period received 30 mg. Cohort 2 received 90 mg followed by 180 mg after a 5-week washout period.

No participant withdrawal occurred, no study stopping criteria were reached, and no serious adverse events (SAEs) were reported. Only two adverse events were classified as possibly related to study treatment: decreased sex drive in 1 participant after the 30 mg dose and an asymptomatic cardiac arrhythmia of low clinical concern detected by Holter monitoring in 1 participant after the 180 mg dose. The cardiac findings were isolated, asymptomatic and not accompanied by laboratory or clinical findings. Both AEs were mild, temporary, self-limiting, and resolved without sequelae.

Adverse events (AEs) that occurred in at least 2 participants (12.5%) and were classified as not related to study treatment included headache and rhinitis, each in 2 participants. All AEs were temporary and fully resolved. Based on the review of the safety data (clinical chemistry, hematology, urinalysis, vital signs, ECG [12-lead and Holter monitoring], physical examination and AEs, Compound A at a strength of up to 180 mg in the fasted state was considered safe and well tolerated by healthy volunteers under the conditions of this trial. Key PK parameters are summarized in TABLE 5. TABLE 6 compares the exposure (Cₘₐₓ and AUC) to the tested doses.

TABLE 5
Key PK parameters of Compound A in healthy volunteers
following oral single dose administration of Compound A

| | | Tₘₐₓ | Cₘₐₓ | AUC₀₋₂₄ | AUC₀₋ₗₐₛₜ | AUC_{0-inf} | T_{1/2} |
|---|---|---|---|---|---|---|---|
| | | (h) | (ng/mL) | (h*ng/mL) | (h*ng/mL) | (h*ng/mL) | (h) |
| | | Geometric Mean (CV %) | | | | | |
| 10 | mg | 8.00* | 131 | 2,380 | 8,180 | 9,560 | 53.5 |
| | | (4.00-8.00) | (41.7) | (47.8) | (45.2) | (43.6) | (36.5) |
| 30 | mg | 4.00* | 200 | 3,520 | 12,400 | 12,900 | 61.7 |
| | | (3.00-12.0) | (77.8) | (75.8) | (67.9) | (65.9) | (29.6) |
| 90 | mg | 8.00* | 472 | 8,430 | 26,300 | 26,800 | 71.3 |
| | | (4.00-8.00) | (64.9) | (67.9) | (53.1) | (52.4) | (20.8) |
| 180 | mg | 8.00* | 1.750 | 27,300 | 73,400 | 75,400 | 76.3 |

| | | | | | |
|---|---|---|---|---|---|
| (4.00-8.00) | (31.5) | (26.3) | (21.1) | (21.4) | (32.9) |

*Shown is median (range)

TABLE 6
Dose proportionality of Compound A in healthy volunteers
following oral single dose administration

| Compared | | Geometric | Geometric | Geometric | Geometric |
|---|---|---|---|---|---|
| doses | Dose | Cₘₐₓ | AUC₀₋₂₄ | AUC₀₋ₗₐₛₜ | AUC_{0-inf} |
| (mg/kg/day) | Ratio | Ratio | Ratio | Ratio | Ratio |
| 180 over 90 | 2.0 | 3.71 | 3.24 | 2.79 | 2.81 |
| 30 over 10 | 3.0 | 1.53 | 1.48 | 1.52 | 1.35 |
| 90 over 30 | 3.0 | 2.36 | 2.39 | 2.12 | 2.08 |
| 180 over 30 | 6.0 | 8.75 | 7.76 | 5.92 | 5.84 |
| 90 over 10 | 9.0 | 3.60 | 3.54 | 3.22 | 2.80 |
| 180 over 10 | 18.0 | 13.36 | 11.47 | 8.97 | 7.89 |

### 3.3: Summary of Potential Risks and Benefits

This Phase 1b/2a study represents the first time Compound A will be administered repeatedly for 28 days to healthy male volunteers. Research in mice, rats, dogs and NHPs suggests that Compound A does not alter testosterone levels, and that it does not affect virility. The impairment of fertility in nonclinical models was shown only after repeat dosing for 3-4 weeks and was reversible after treatment discontinuation in three out of four species: mouse, dog and NHP with the rat being a clear outlier (see Section 3.1.4). Out of precaution, only men who have decided to have a vasectomy and are waiting for the procedure, or men who, in the opinion of the investigator, have made a firm decision not to father children in the future will be enrolled in this Phase 1b/2a clinical trial. As an additional safety measure, a sperm sample from all participants will be collected and cryopreserved at no cost before administering the investigational product until their last study visit, and for 10 years thereafter.

Repeat daily dosing of ≥125 mg/kg/day in the rat and ≥10 mg/kg/day in the dog were not tolerated resulting in death and morbidity. These doses convert to respective Human Equivalent Doses (HEDs) of ≥1,612.8 mg and ≥444.8 mg fixed dose based on 80 kg body weight. In the rat at non-tolerated doses, the hematopoietic, hepatic, adrenal and lymphoid systems were affected. In the dog, the cause of death and moribund condition was attributed to renal toxicity with additional pathological changes in multiple organ systems considered potentially secondary to the renal changes. While such changes are not expected with repeated dosing at the proposed dose ranges (15-180 mg), standard safety monitoring measures are part of the study and include monitoring liver toxicity (through regular laboratory analyses), renal toxicity (through regular laboratory analyses, physical exams and urinalyses), cardiac toxicity (through ECGs, laboratory analyses and physical exams) and endocrine toxicity (through laboratory analyses and physical exams). Safety laboratory analyses include serum chemistry, hematology, coagulation, and lipid panel.

Compound A has no genotoxic potential based on findings from an in vitro bacterial reverse mutation (Ames) test, a chromosomal aberration assay, and an in vivo rat micronucleus study. Direct in vitro inhibition of CYP1A2, CYP2B6, CYP2C8, CYP2C9, CYP2C19, CYP2D6 and CYP3A4/5 (midazolam and testosterone as substrates) was observed for Compound A with respective IC50 values of 30.1, 6.10, 8.37, 14.6, 85.1, >100, 58.6, and 53.6 μM. Compound A had NADPH-independent time-dependent inhibition against CYP2D6 and CYP3A4/5, and no TDI potential against CYP1A2, CYP2B6, CYP2C8, CYP2C9, and CYP2C19 over a concentration range of 0.3 to 100 μM. However, the likelihood of drug interactions is low since healthy participants who meet eligibility criteria will be enrolled in this Phase 1b/2a clinical trial. An allowance for select concomitant medications may be considered for select participants at the discretion of the investigator and/or sponsor if drug profiles are not anticipated to interact with Compound A. Such medications may include, but will not be limited to paracetamol or ibuprofen as assessed by both the PI and the Sponsor. Exceptions may apply, as determined by the investigator, if each of the following criteria are met: medication with a short half-life if the washout is such that no PD activity is expected by the time of dosing with IMP; and if the use of medication does not jeopardize the safety of the trial participant; and if the use of medication is not considered to interfere with the objectives of the study. Additional precautions to avoid grapefruit, pomegranates, star fruit, or Seville oranges for 7 days prior to admission, and juices and products containing these fruits for the duration of the study will be taken.

Based on results from an in vitro molar extinction coefficient assay, Compound A has the potential to cause phototoxicity from UV light and further testing for potential phototoxicity is needed. As a result, participants will be advised to minimize exposure to sunlight, wear sunglasses and clothing that minimizes exposure to sunlight, and use sun cream on exposed areas when going outdoors during the 28-Day dosing period and until 10 days post last dose.

Collecting a blood sample from a vein may cause pain, swelling, bruising, light-headedness, fainting, and very rarely, clot formation, nerve damage and/or infection at the site of the needle stick.

During cannulation, more than one attempt may be needed to insert the cannula in a vein of a participant, and it is possible that bruising and/or inflammation may be experienced at the site of cannulation.

Electrocardiogram (ECG) stickers on the participants' chests and limbs may cause some local irritation and may be uncomfortable to remove but participants will be closely monitored to ensure any local irritation does not persist.

### 4: Objectives

### 4.1: Primary Objective

The primary objective is to assess the safety and tolerability of repeated daily oral dosing of Compound A for 28 days.

### 4.2: Secondary Objectives

The secondary objectives are:

To evaluate the pharmacodynamics (PD) of Compound A.

To assess the pharmacokinetics (PK) of Compound A.

### 4.3: Exploratory Objectives

The exploratory objectives are:

To evaluate the effect of Compound A on change from baseline corrected QT interval in ECG (QTc) interval.

To evaluate the effect of Compound A on inflammatory biomarkers.

### 5: Study Design

### 5.1: Overall Study Design and Plan

Study is a Phase 1b/2a, open label, dose escalation study to evaluate the safety, tolerability, PK, and PD of repeated daily dosing of Compound A for 28 days. The design, study procedures and schedule of assessments of the study are described in Section 5.1.1 and Section 5.1.2, respectively, and Section 1.1.

In the Phase 1b part, 4 dosing cohorts and one optional 5th cohort with 4 participants each will be evaluated. All participants will receive Compound A. Participants may be replaced automatically if prior to any dosing. If a participant is required to be replaced post-dose, the replacement may be approved at discretion of Sponsor and PI to ensure available data in at least 3 evaluable participants per cohort. In the Phase 2a part, up to 3 cohorts will receive doses within the range of doses that was well tolerated and had biological activity in the Phase 1b portion. Each Phase 2a cohort will have up to 10 participants. All participants will receive Compound A. Participants who discontinue early may be replaced. All participants will be dosed following fasting conditions.

Participants of the Phase 1b part will be admitted to an in-patient clinical research unit in the morning of the day before dosing (Day −1) and participants will remain resident until Day 7 (day of discharge). Participants of the Phase 2a part will be admitted to an in-patient clinical research unit in the morning of the day before dosing (Day −1) and will remain resident until Day 2 (day of discharge). Participants of the Phase 1b and 2a parts will be readmitted to the in-patient clinical research unit in the morning of Day 27 (day before last dosing) and will remain resident until 72 h post-last dose (Day 31, day of discharge).

The participants will undergo vital signs measurements, ECGs (12-lead and Holter monitoring), laboratory tests (which will include a comprehensive metabolic panel and complete blood count with differential lipid panel) and will provide semen samples. While participants are resident in the clinical research unit (Day 28), they will be escorted to the fertility clinic after dosing and collection of all other assessments, and will be escorted back to the clinical research unit after providing a semen sample. During the screening period and on Days 21, 42, 56, 84, 112, 140 and 168, they will attend the fertility clinic after collection of assessments at the clinical research unit. On Day 196, and—if necessary—on Days 224, 252 and 280, participants will attend the fertility clinic themselves before collection of all other assessments. Further visits to the fertility clinic will be arranged for individual participants at the investigator's discretion until sperm parameters return to baseline, or are within the WHO recommended levels, or the investigator considers that further monitoring is not clinically indicated, or the participant is lost to follow-up. Laboratory tests performed during the study will be reviewed by an investigator within 48 hours after results become available. After at least 3 evaluable participants in a cohort of the Phase 1b part complete the 28-day dosing, safety data and available PK and PD data from up to Day 28 will be reviewed by the Dose Escalation Review Committee (DERC) to determine the dose level for the subsequent cohort.

After the review of the safety, and available PK and PD data from up to Day 28 of at least 3 evaluable participants of the last cohort of the Phase 1b part, the Phase 2a part of this study will be initiated. The doses for the Phase 2a cohorts will be selected based on emerging data from the Phase 1b part.

The following assessments and procedures will ensure the safety of the participants during the study:

Twelve-lead and Holter ECG monitoring, and troponin assessments will be performed to detect any potential cardiac issues.

Vital sign assessments will be performed, including heart rate, blood pressure (BP) and body temperature.

Clinical laboratory tests will be performed at the scheduled time points.

Any clinically significant changes in the assessments will be discussed immediately with the Medical Monitor. Participants will be informed if they should discontinue study treatment.

Participants will complete a psychosexual diary during the study to assess any changes in psychosexual parameters. Changes are not expected as Compound A impacts spermatogenesis non-hormonally.

Participants who withdraw from the study should undergo all End of Trial Visit procedures, including all safety assessments. Participants will be questioned about how they have been feeling and any adverse events (AEs) they have experienced. All AEs and SAEs will be reported and recorded appropriately.

A DERC will be formed to review data, give dose escalation recommendations, and approve the next dose level. The DERC will comprise the Investigator, the Study medical monitor and Sponsors medically qualified representative who is familiar with the study protocol and IB, science lead and PK and statistical experts where appropriate.

### 5.1.1: Multiple Ascending Dose (Phase 1b Part)

In the Phase 1b part of the study, 4 dosing cohorts and one optional 5th cohort with 4 participants will be evaluated (up to 20 participants): 15, 45, 90 and 180 mg (TABLE 7). These are planned doses that may be amended based on emerging safety, PK and PD data. Cohort 1 participants will receive study drug under fasting conditions. Cohort 2-5 participants will receive study drug under fasting or fed conditions.

TABLE 7

| Cohort | IMP | Planned Dose | | Route of Administration |
|---|---|---|---|---|
| 1 | Compound A | 15 | mg | Oral, Fasted |
| 2 | Compound A | 45 | mg | Oral^{b} |
| 3 | Compound A | 90 | mg | Oral^{b} |
| 4 | Compound A | 180 | mg | Oral^{b} |
| 5 (optional) | Compound A | X | mg^{a} | Oral^{b} |

^{a}The selected dose will depend on emerging safety and PK data.
^{b}The dose will be administered with or without food.

The starting dose for Compound A in the Phase 1b multiple ascending dose (MAD) part is 15 mg (see Section 5.3 for rationale for dose selection).

Participants will fast (except water) for at least 10 hours overnight prior to administration of study drug (Compound A), and for at least 2 hours after dosing.

Laboratory data will be examined on an ongoing basis by the Principal Investigator and/or Medical Monitor for any signs of drug induced liver injury (DILI).

The DERC will evaluate safety, tolerability and available PK and PD data from at least 3 evaluable participants from up to Day 28 to determine the dose level for the subsequent cohort.

The specific scope, roles, membership, decision making timepoints, and decision-making processes of the DERC will be outlined in the DERC Charter. All decisions to repeat a dose level, proceed to the next dose level or de-escalate the dose will be properly recorded in a source document. These decisions will be made in agreement with the Sponsor.

### 5.1.2: Cohort Expansion (Phase 2a Part)

In the Phase 2a part, up to 3 cohorts of up to 10 participants (up to 30 participants) will receive doses within the range of doses that was well tolerated and had biological activity in the Phase 1b portion.

The expansion of up to 3 cohorts will be initiated after the review of the safety, and available PK and PD data from up to Day 28 of at least 3 evaluable participants of the last cohort of the Phase 1b part. These cohorts will be selected based on emerging data from the Phase 1b part.

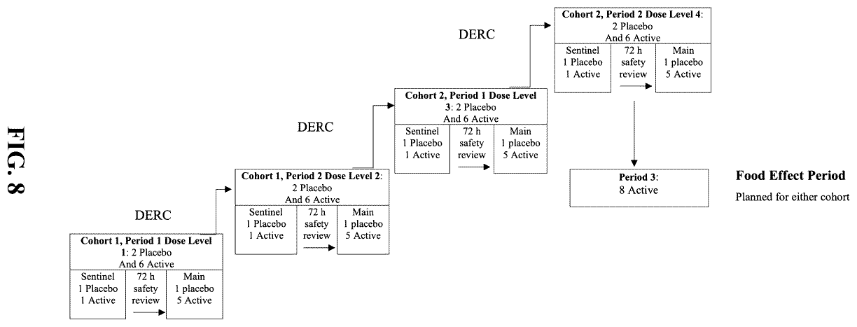

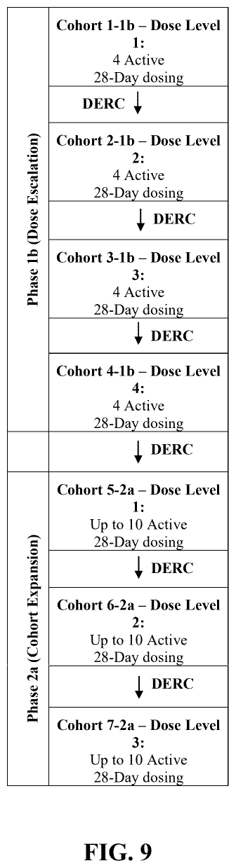

FIG. 9 is a flow chart of the study design for Dose Escalation (Phase 1b Part) and Cohort Expansion (Phase 2a Part), according to an embodiment of the present disclosure. DERC=Dose Escalation Review Committee. In the Phase 1b part, four (4) dosing cohorts and one optional 5th cohort with 4 participants will be evaluated (up to 20 participants). In the Phase 2a part, up to 3 cohorts of up to 10 participants (up to 30 participants) are planned. All participants will receive Compound A with or without food.

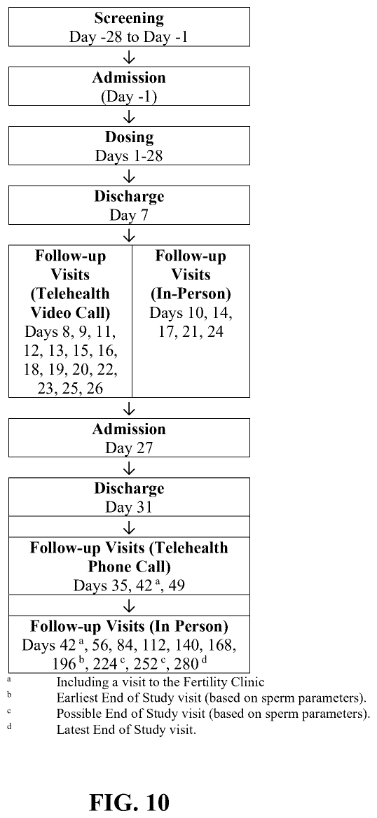

FIG. 10 is a flow chart of Study Sequence per Cohort in the Phase 1b Part, according to an embodiment of the present disclosure.

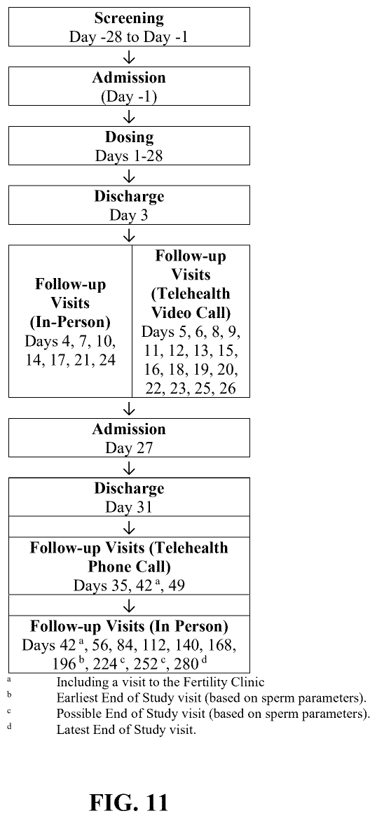

FIG. 11 is a flow chart of Study Sequence per Cohort in the Phase 2a Part, according to an embodiment of the present disclosure.

### 5.2: Rationale and Discussion of Study Design

This is the first human repeat dose study of Compound A, a small molecule being developed as a male non hormonal contraceptive. In preclinical studies, no impact on sperm parameters was observed with single doses of Compound A as high as 1,000 mg/kg in rats (HED 161.29 mg/kg). The pharmacological on target effect-reduction of sperm counts—of Compound A in nonclinical models was only shown after repeat dosing for 3-4 weeks.

Out of precaution, only men who have decided to have a vasectomy and are waiting for the procedure, or men who, in the opinion of the investigator, have made a firm decision not to father children in the future, will be selected as the study population. As an additional safety measure, a sperm sample from all participants will be collected and cryopreserved at no cost before administering the investigational product until their last study visit, and for 10 years thereafter.

Safety data, and PD and PK data (where available) will be evaluated before proceeding to the next dose level.

Vital signs, ECGs (12-lead and Holter monitoring), and clinical laboratory tests will be performed to monitor the well-being of participants. Participants will also be closely monitored for any signs of liver, endocrine, cardiac or kidney impairment as well as changes in blood parameters.

Pharmacodynamic endpoints have been selected to evaluate effects on spermatogenesis, spermiation, and sperm motility. Semen samples will be collected from all participants 3 times during the screening period, on Days 21 and 28 during the dosing period, and on Days 42, 56, 84, 112, 140, 168 and 196. If sperm parameters have not returned to at least the 5th percentile of WHO ranges of normality (WHO, 2010; ≥15 million sperm cells/ml, ≥39 million/sperm cells in total ejaculate, ≥32% progressive motility, ≥4% normal forms) by Day 196 (earliest End of Study visit; approximately 2.5 spermatogenic cycles), semen samples will also be collected until the resolution of any clinically relevant changes in their sperm parameters (as assessed at visits on Days 224, 252, and 280+/−2 days). The last possible End of Study visit will be Day 280+/−2 days. Concentrations of Compound A will also be assessed in semen samples. Based on previous preclinical NHP studies it is expected that Compound A will not be detectable in semen samples. Blood samples for PK assessments will be collected throughout the study.

The study design, protocol procedures, schedule of assessments and decision-making procedures are aimed to protect the participants' safety and generate quality data that will inform the potential future clinical development of Compound A.

### 5.3: Selection of Doses in the Study

The proposed starting dose for Compound A in the Phase 1b multiple ascending dose MAD part of this study is 15 mg. This dose has been selected based on safety and tolerability data from the first in human Phase 1a study where single doses of Compound A from 10 mg to 180 mg were found to be safe and well tolerated (summarized in Section 3.2) and on the observed NOAELs from the following 3 repeat-dose studies (see TABLE 8).

Rat 28-day repeat dose GLP study in which animals were actually dosed for 42 days in total (28-day dosing followed by 14-day mating period with continued dosing) with a NOAEL of 30 mg/kg/day.

Dog 28-day repeat dose GLP study with a NOAEL of 5 mg/kg/day.

NHP 14-day repeat dose non-GLP study with a NOAEL of 10 mg/kg/day.

TABLE 8
Summary of repeat-dose NOAELs and
respective HEDs of Compound A

| | Rat | | Dog | | NHP | |
|---|---|---|---|---|---|---|
| NOAEL | 30 mg/kg/day ^{b} | | 5 mg/kg/day | | 10 mg/kg/day | |
| | (42 days) | | (28 days) | | (14 days) | |
| HED^{a} (based | 4.8 | mg/kg | 2.8 | mg/kg | 3.2 | mg/kg |
| on BSA) | | | | | | |
| HED for 60 kg BW | 288 | mg | 168 | mg | 192 | mg |
| HED (60 kg BW) with | 29 | mg | 17 | mg | 19 | mg |
| 10X safety factor | | | | | | |
| HED for 80 kg BW | 384 | mg | 224 | mg | 256 | |
| HED (80 kg BW) with | 38 | mg | 224 | mg | 26 | mg |
| 10X safety factor | | | | | | |

^{a}HED was calculated by dividing the animal dose by the following FDA conversion factors: 6.2 (rat); 1.8 (dog); 3.1 (NHP)
^{b} Highest dose tested.
BSA = body surface area; BW = body weight; HED = human equivalent dose; NHP = nonhuman primate; NOAEL = no-observed-adverse-effect level.

The starting dose has also been assessed relative to the minimal anticipated biological effect level (MABEL) after repeat dosing in 3 biologically relevant species:

Dog 28-day repeat dose GLP study with a MABEL of 2.5 mg/kg/day.

Rat 35-day repeat dose non-GLP study with a MABEL of 0.75 mg/kg/day.

NHP 49-day repeat dose non-GLP study with a MABEL of 2.5 mg/kg/day.

MABELs and HEDs based on body surface area (BSA) on the basis of mg/kg and fixed dose on the basis of 60 or 80 kg body weight are listed in TABLE 9.

TABLE 9
Summary of repeat-dose MABELs and
respective HEDs of Compound A

| | Rat | | Dog | | NHP | |
|---|---|---|---|---|---|---|
| MABEL | 0.75 mg/kg/day | | 2.5 mg/kg/day | | 2.5 mg/kg/day | |
| (days of dosing) | (35 days) | | (28 days) | | (49 days) | |
| HED^{a} (based on BSA) | 0.1 | mg/kg | 1.4 | mg/kg | 0.8 | mg/kg |
| HED for 60 kg BW | 7.2 | mg | 84 | mg | 48 | mg |
| HED for 80 kg BW | 9.6 | mg | 112 | mg | 64 | mg |

^{a}HED was calculated by dividing the animal dose by the following FDA conversion factors: 6.2 (rat); 1.8 (dog); 3.1 (NHP)
BSA = body surface area; BW = body weight; HED = human equivalent dose; MABEL = minimal anticipated biological effect level; NHP = nonhuman primate.

Based on a body weight of 80 kg, the proposed starting dose of 15 mg is 0.6-fold above the HED of the MABEL in the rat, 7.4-fold below the HED of the MABEL in the dog and 4.3-fold below the HED of the MABEL in the NHP.

The proposed dose escalation for Compound A in the Phase 1b MAD part of the study is as follows:
Cohort 1:15 mg Compound A
Cohort 2:45 mg Compound A
Cohort 3:90 mg Compound A
Cohort 4:180 mg Compound A

Dose escalation between dose levels will be a maximum 3-fold. These doses may be amended based on emerging PK and safety data.

At this stage of development, it is still difficult to predict the range of doses and the dose regimens that could provide optimal efficacy, reversibility, and adequate tolerability. Considering the non-GLP NHP repeat dose study findings as the closest model to human biology, a potential efficacious dose could range from 2.5 to 7.5 mg/kg/day (equivalent to HEDs ranging from 0.81 to 2.42 mg/kg/day) on repeat dose administration. Considering that the average weight for male adults in Oceania is around 70-80 kg (Walpole et al., 2012), this could translate to doses be-tween 57-170 mg/day (70 kg BW) and 65-194 mg/day (80 kg BW), which covers the range of doses planned to be explored in the 28-day repeated dose escalation study (15-180 mg). As more information is gathered on the safety, PK and PD of Compound A it will be reasonable to consider and explore if higher doses given less frequently could also result in similar safety and efficacy with added convenience compared to lower daily doses.

### End of Study Definition

The clinical study will be considered completed when the final participant's last follow-up visit has occurred (between Days 196 and 280). If the study is terminated prematurely, the study ends when the sponsor notifies the investigator in writing that the trial has ended or when the last participant completes the final follow-up, whichever is later. Any changes to this definition will be notified as a substantial amendment.

Medsafe and Therapeutic Goods Administration (TGA) will be notified of the conclusion of the study within 90 days of the end of the study, or within 15 days if the study is terminated early, clearly explaining the reasons for the termination.

### Stopping Criteria

The study will be halted, and the risk to other participants evaluated if any of the following criteria are met:

A serious adverse reaction (i.e., a SAE considered at least possibly related to the IMP administration) in one participant.

Severe non-serious adverse reactions (i.e., severe non-serious AE considered as, at least possibly related to the IMP administration) in two participants in the same cohort, independent of within or not within the same system organ class.

Relatedness to IMP will be assessed by the investigator.

If the study is halted due to the stopping rules above, a temporary halt will be submitted to Medsafe and TGA in the form of a substantial amendment. The study may be resumed or terminated; however, it will not be resumed until a further substantial amendment to resume the study is submitted and approved by Medsafe or TGA.

### Dose Escalation Stopping Rules

Further enrollment and study drug administration will be investigated and will be stopped based on dose-limiting AEs, as described below:

One or more participants experience a possibly IMP-related serious adverse event (SAE).

Two or more participants experience a possibly IMP-related severe or clinically significant non-serious AE.

Any liver function abnormalities that meet the definition of Hy's Law

ALT or AST elevation >3 ULN

Total bilirubin >2×ULN without findings of cholestasis (elevated alkaline phospha-tase)

No other possible causes of aminotransferases elevation and hyperbilirubinemia including but not limited to viral hepatitis or administration of other drugs known to be hepatotoxic.

If all participants within a cohort experience a clinically relevant decrease from a normal base-line in sperm parameters on Day 28 that fall below the 2.5th percentile of WHO range of normality (WHO, 2010), a maximally 3-fold higher dose level may be explored to further evaluate safety and tolerability.
<9 million sperm cells/ml
<23 million/sperm cells in total ejaculate
<28% progressive motility
<3% normal forms.

Discontinuation of study drug does not mean discontinuation from the study, and remaining study procedures should be completed as indicated by the study protocol.

### 7: Treatment

Compound A will be administered orally in Swedish orange size 0 hard gelatin capsules.

TABLE 10
Investigational Medicinal Product

| | | Route of |
|---|---|---|
| IMP Name | Planned Dose | Administration |
| Compound A Capsule, | 15 to 180 mg | Oral, Fasted |
| 15, 45 and 90 mg | | |

The Compound A capsule is an investigational product for use only in the proposed clinical trial. Only participants enrolled in the study may receive study treatment and only authorized site staff may supply or administer study treatment. Quotient Sciences is manufacturing the IMP, and the suitability of the manufacturing process will be documented in a Pharmaceutical Development and Control Strategy Report. IMP will be reconciled and destroyed in accordance with the study-specific plan.

### 7.2: Selection of Timing of Dose for Each Participant

Dosing of participants will start on Day 1. Study drug (Compound A) will be administered after an overnight fast of at least 10 hours with 240 mL of water. Participants should also fast for at least 2 hours after dosing.

Water will be allowed ad libitum during the study, except for the time period between 1 hour before dosing and 1 hour after dosing, when the only water permitted will be the water administered with the study drug. Decaffeinated fluids will be allowed ad libitum from no earlier than 2 h post-dose.

A late breakfast or morning snack should be taken no earlier than 2 h post-dose. Lunch should be taken at approximately 4 h post-dose, an evening meal at approximately 10 h post-dose, and an evening snack should be finished no later than 12 h post-dose.

When study drug will be administered with food, participants will fast for at least 10 hours overnight, and will then be provided a high-fat, high-calorie breakfast, in accordance with FDA guidance. The breakfast should be consumed over a maximum period of 25 minutes, with dosing occurring 30±5 minutes after the start of breakfast. Participants should be encouraged to eat their meal evenly over the 25-minute period. It is acknowledged that some participants will take less time to eat, but dosing should still occur 30±5 minutes after the start of breakfast. The majority of the meal (>90%) should be consumed.

### 7.3: Dose Adjustment Criteria

Dose escalation (Phase 1b part) and dose selection for cohort expansion (Phase 2a part) is planned, as described in Section 5.1.1 and Section 5.1.2, respectively. Safety, PK, and PD data of up to Day 28, where applicable, will be evaluated for dosing decisions for:

Dose selection for the next dose level.

Changes to safety and/or PK time points, if there is reason to believe that the change might im-prove the quality of the data or the safety of the participants as a consequence of review of emerging data.

### 7.3.1: Criteria for Dose Escalation (Phase 1b Part)

Progression to the next dose level will be permitted if the review of safety, PK and sperm PD data (where available) of up to Day 28 in at least 3 evaluable participants suggests that it is safe to do so. Sperm PD data will be reviewed for efficacy (reduction of sperm count and/or sperm motility as the anticipated pharmacological effect).

Data provided by the clinical research units will be provided to the DERC in accordance with their respective standard operating procedure (SOP) on interim dose decision making and dose escalation. The decision will be documented and signed by the investigator as per clinical re-search unit's current SOP. Evidence of the decision will be retained in the Investigator Site File (ISF).

For dose escalation to proceed, data from up to Day 28 must be available from a minimum of 3 evaluable participants who have completed the planned safety assessments, and planned PK and PD assessments (where available). Dose increases will only be made after a complete review of all data collected from the previous dose group by the DERC. The following data are required:
AEs (mandatory)
Concomitant medications (if used)
Vital signs
Clinical laboratory results, including serum hormones
ECGs
Physical examinations
Plasma concentrations of Compound A
Interim PK parameter estimations (Cmax, Tmax, AUC)
Interim blood PD parameters estimations
Semen and sperm parameter data

Rules for dose decision are as follows:

Dose escalation between MAD periods will be a maximum 3-fold.

Sperm PD data will only be reviewed for efficacy (reduction of sperm count and/or sperm motility as the anticipated pharmacological effect).

If ≥75% of participants within a cohort- or the lowest numerical approximation of the calculated percentage-experience a clinically relevant decrease from a normal baseline in sperm parameters on Day 28 that fall below the 2.5th percentile of WHO range of normality (WHO, 2010), the dose will be escalated to the next planned dose level.
<9 million sperm cells/ml
<23 million/sperm cells in total ejaculate
<28% progressive motility
<3% normal forms.

If 100% of participants within a cohort experience a clinically relevant decrease from a normal baseline in sperm parameters on Day 28 that fall below the 2.5th percentile of WHO range of normality (WHO, 2010), a maximally 3-fold higher dose level may be explored to further evaluate safety and tolerability.

If, following review by the DERC it is deemed acceptable to continue dose escalation above the proposed maximum dose (180 mg), a substantial amendment with relevant data will be submit-ted for approval to Medsafe or TGA.

Dosing decisions should be made in consultation with the Sponsor. The decision and all available safety, PK, and PD information will be documented.

Doses will be flexible during the study to allow for increases or reductions in the planned MAD escalation based on the safety and available PK and PD data from the preceding dosing period. When it is not appropriate to escalate the dose, then the same dose, a previously administered dose, or an intermediate dose may be given following DERC recommendations and further discussion with the Principal Investigator and Sponsor.

The dosing decisions and all information available will be documented and included in Clinical Study Report (CSR) Appendices after completion of the study.

### 7.3.2: Criteria for Dose Selection for Cohort Expansion (Phase 2a Part)

Dose levels will be selected based on emerging data from the Phase 1b part.

### 7.4: Treatment Compliance

Participants will be counseled on the importance of adhering to study procedures. In the Phase 1b part, study drug will be administered at the designated clinical research unit on Days 1-7 under the supervision of trained site staff, who will ensure compliance with study drug administration procedures. On Days 8, 9, 11, 12, 13, 15, 16, 18, 19, 20, 22, 23, 25 and 26, participants will self-dose at home under supervision with a telehealth staff member via video call. On Days 10, 14, 17, 21, 24, 27 and 28, participants will self-dose at the designated clinical research unit under the supervision of trained site staff. In the Phase 2a part, study drug will be administered at the designated clinical research unit on Days 1-3 under the supervision of trained site staff, who will ensure compliance with study drug administration procedures. On Days 4, 7, 10, 14, 17, 21, 24, 27 and 28, participants will self-dose at the designated clinical research unit under the supervision of trained site staff. On Days 5, 6, 8, 9, 11, 12, 13, 15, 16, 18, 19, 20, 22, 23, 25 and 26, participants will self-dose at home under supervision with a telehealth staff member via video call.

Mouth and hand checks will be conducted after dosing to ensure the capsules have been swallowed.

### 7.5: Permitted and Prohibited Therapies

Concomitant medication use will be monitored by the Principal Investigator or designee during the sample collection period within the study and during any necessary washout periods. All concomitant medications used (including over-the-counter medications and herbal supplements) will be recorded in the source document and on the appropriate eCRF.

### Permitted Therapies

Male participants with female partner(s) of childbearing potential will be advised to use contraception including condom contraception from first administration of IMP until Day 56 (see Section 6.2.4). Permitted therapies include up to 4 g of paracetamol or up to 3.2 g of ibuprofen per day and medications deemed necessary by the investigator to treat AEs. Other accepted concomitant medications may be accepted at the discretion of both the PI and the Sponsor. COVID-19 vaccines are accepted concomitant medications. It is preferable for participants not to receive the vaccine within 72 h prior to administration of IP, where possible, so that by the time of dosing any effects of the vaccine (e.g., pyrexia, fatigue, pain/stiffness at site of injection) are likely to have abated; however, participants would not be excluded on this basis.

### Prohibited Therapies

No prescribed, over-the-counter medication or vitamins/herbal remedies supplements will be permitted from 14 days before IP administration until 28 Days after the last administration of study drug on Day 28. Participants may be excluded from the study if they are taking a medication that may, in the opinion of the investigator, affect the participant's well-being, the study conduct, or the interpretability of the results.

### 8: Study Procedures

### Primary Endpoints

The primary endpoint is the safety profile including the incidence of AEs, safety laboratory tests (clinical chemistry and liver function tests), CBC with differential, and lipid panel, ECGs (12-lead and Holter monitoring), vital signs, and other safety assessments following repeated daily oral dosing of Compound A for 28 days.

### Secondary and Exploratory Endpoints

The secondary endpoints are as follows:

Primary PD parameters of Compound A: semen parameters including ejaculate volume, ejaculate pH, total sperm count per ejaculate, sperm concentration, total motility [progressive, non-progressive, immotile], and sperm morphology (except vasectomized participants).

Plasma PK parameters (area under the curve from time 0 extrapolated to infinity [AUCinf], area under the curve from time 0 to the last measured concentration

[AUC0-t], area under the curve from time 0 to 24 hours [AUC0-24], time to Cₘₐₓ [Tₘₐₓ], T1/2, lag time [T_{lag}], apparent volume of distribution [Vz/F], oral clearance [CL/F], and Cₘₐₓ) of Compound A, including the effect of food on the PK.

### Compound a Concentrations in Semen

Secondary PD parameters of Compound A, including serum hormones (FSH, LH, T and calculated free T), and SHBG.

The exploratory endpoints are as follows:

Formal statistical analysis of relationship between the change from baseline in QTc interval using Bazett's and Fridericia's formulation (AQTcF) and plasma Compound A concentration.

Formal statistical analysis of AQTcF at each post-dose timepoint for the comparison between each active dose level and placebo.

Exploratory PD parameters (inflammatory biomarkers) of Compound A including tumor necro-sis factor (TNF)-α, interleukin (IL)-6, high-sensitivity C-reactive protein (hsCR), E-selectin, and von Willebrand factor (vWF).

### 10.3.2.1 Pharmacokinetic Assessments

Venous blood samples will be withdrawn via an indwelling cannula or by venipuncture at the time points detailed in the schedule of assessments.

The acceptable deviations from the nominal blood sampling times are as follows:

The pre-dose samples will be taken at −0.5 h before dosing.

0 to 1 h post-dose samples will be taken within +2 min of the nominal post-dose sampling time.

2 to 12 h post-dose samples will be taken within +10 min of the nominal post-dose sampling time.

18 h to 48 h post-dose samples will be taken within +30 min of the nominal post-dose sampling time.

96 to 336 h post-dose samples will be taken within +2 h of the nominal post-dose sampling time.

Semen PK samples will be collected once during the screening period (at least 5 days prior to dosing), on Days 21, 28, 56, and—if Compound A is present in semen on Day 56—also on Day 84. Sample collection should occur between 2 days and 5 days after the last ejaculation.

Samples will be collected as described in the clinical sample processing manual.

### 10.3.2.2 Pharmacodynamic Assessments

Semen PD samples will be collected 3 times during the screening period, on Days 21 and 28 during the dosing period, and on Days 42, 56, 84, 112, 140, 168 and 196. If sperm parameters have not returned to at least the 5th percentile of the WHO range of normality (WHO, 2010; ≥15 million sperm cells/ml, ≥39 million/sperm cells in total ejaculate, ≥32% progressive motility, >4% normal forms) by Day 196 (earliest End of Study visit), semen samples will also be collected until the resolution of any clinically relevant changes in their sperm parameters (as assessed at visits on Days 224, 252, and 280+/−2 days). The last possible End of Study visit will be Day 280+/−2 days.

Participants will have serum hormones (T, FSH, LH, E2 and calculated free T), and SHBG samples drawn once during the screening period, at −0.5 hours pre-dose (Day 1), and on Days 3, 7, 14, 21, 28, 56 and 196 (earliest End of Study Visit); if necessary also on Days 224 or 252 (possible End of Study visits) or on Day 280 (latest End of Study visit).

Blood samples for evaluating general inflammatory biomarkers and biomarkers of vascular endothelial injury (TNF-α, IL-6, hsCR, E-selectin, and vWF) will be collected once during the screening period, at −0.5 hours pre-dose (Day 1), and on Days 3, 7, 14, 21, 28, 56 and 196 (earliest End of Study Visit); if necessary, also on Days 224 or 252 (possible End of Study visits) or on Day 280 (latest End of Study visit).

The acceptable deviations from the nominal blood sampling times are as follows:

The pre-dose samples will be taken ≤1 h before dosing.

24 h post-dose sample will be taken within +30 min of the nominal post-dose sampling time.

For return visits on Days 56, 196, 224, 252 or 280, the sample will be taken ±2 hours from the nominal return visit time point.

### Adverse Events of Special Interest

Adverse events of special interest (AESIs) were selected based on available nonclinical safety information. The following events will be considered AESIs:

Prolongation of QTc (>500 msec or >70 msec above baseline)

Protracted (>1 hour) nausea and vomiting without an alternative explanatory cause.

Any related AE deemed by the investigator to be dose-limiting (see Section 6.5) or Common Terminology Criteria for Adverse Events (CTCAE version 5.0) Grade 3 and above.

Clinical investigations including signs of DILI based on following criteria from FDA guidance:
ALT or AST >8× upper limit of normal (ULN)
ALT or AST >5×ULN for more than 2 weeks
ALT or AST >3×ULN and (total bilirubin >2×ULN or international normalized ratio >1.5)
ALT or AST >3×ULN with the appearance of fatigue, nausea, vomiting, right upper quadrant pain or tenderness, fever, rash, and/or eosinophilia (>5%)

Clinically significant changes in complete blood count, coagulation, and blood chemistry

Clinically significant changes from baseline in renal function

### Example 3: Phase 1B/2A Study

Open label dose escalation study to evaluate safety, tolerability, pharmacokinetics, and pharmacodynamics of repeated daily oral dosing of Compound A for 28 days or 90 days in healthy men

### Updates From Example 2 Clinical Protocol:

Extending the dosing phase from 28 days to 90 days and splitting this study into 3 parts

Dosing men for 4 weeks with Compound A at doses of up to 90 mg was not sufficient to decrease sperm counts. The main underlying reason provided by experts in the male contraceptive field is the dosing duration. Dosing for 28 days in men only covers approximately one third of the entire cycle from sperm production to sperm release, which takes 85 days in men (71 days for one spermatogenic cycle and 14 days for epididymal transit). Clinical trials with hormonal methods demonstrate that it takes a median time of 2.73 months to suppress sperm concentrations below fertile limits. Therefore, the dosing period will be extended from 28 days to 90 days with the anticipation that this will show the intended effect (reduction in sperm concentration).

Various sections have been updated throughout to incorporate changes related to extending the dosing phase to 90 days and splitting this study into 3 parts: a 28-day Phase 1b part (Part 1), a 90-day Phase 1b part (Part 2) and a 90-day Phase 2a part (Part 3).

### Rat Recovery

Sections 5.1.1 (Efficacy), 5.1.4 (Repeat Dose Toxicity) and 5.3 (Summary of Potential Risks and Benefits):

These sections were updated to clarify that rats recover from disruption of spermatogenesis after 28-day dosing with Compound A at 0.75 mg/kg/day and 1 mg/kg/day indicating that all 4 animal species-mouse, rat, dog and non-human primate-recover from the intended pharmacological effect.

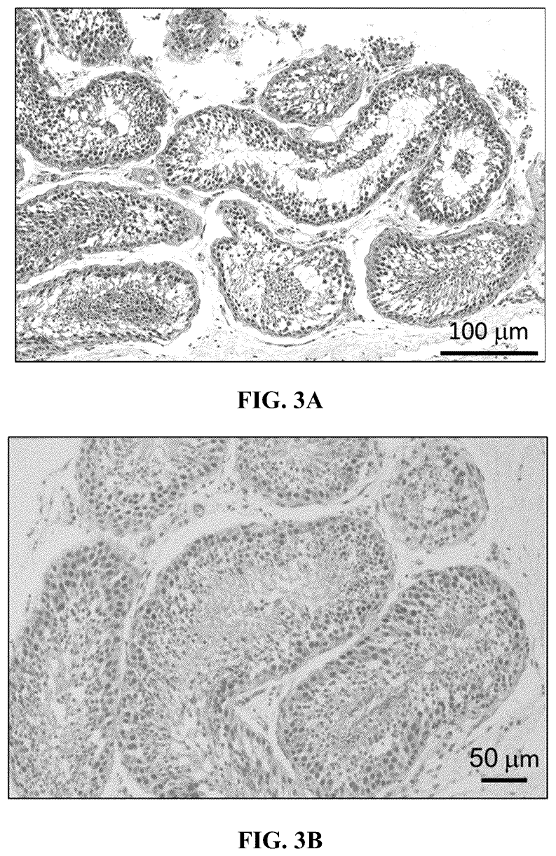

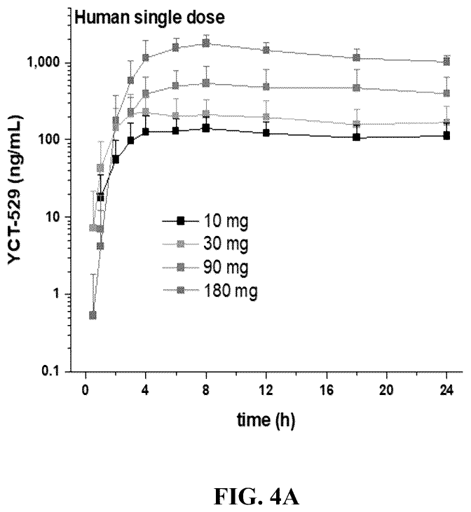

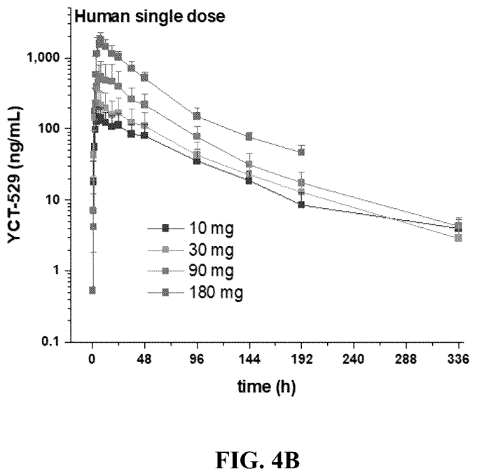

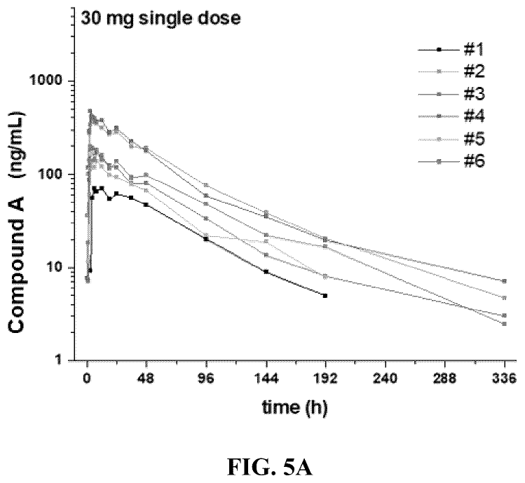

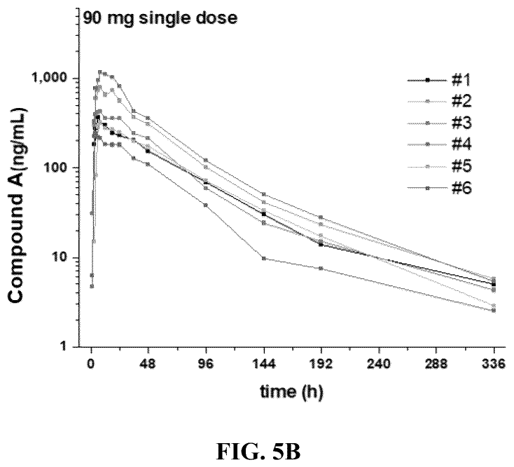

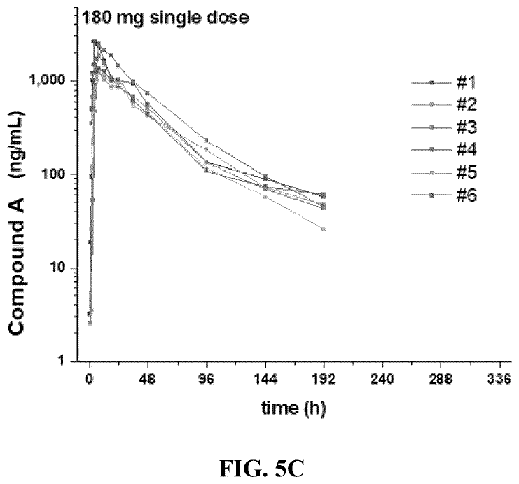

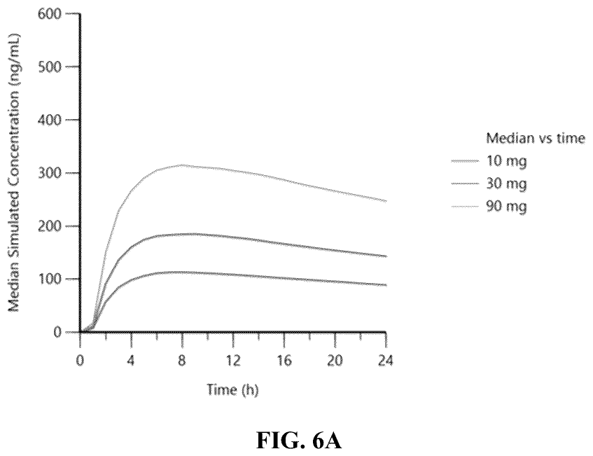

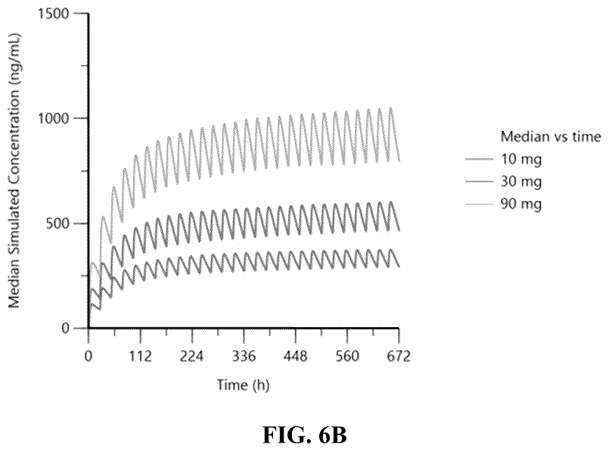

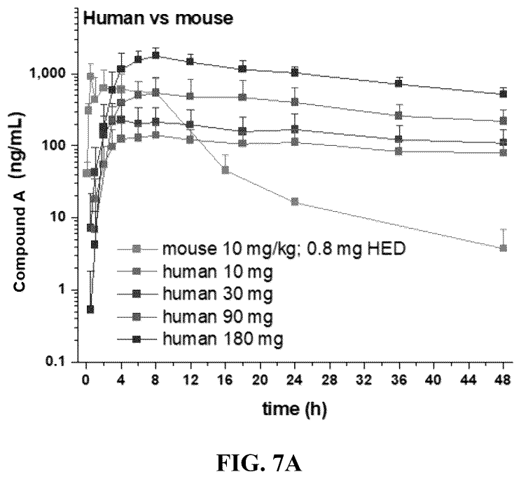

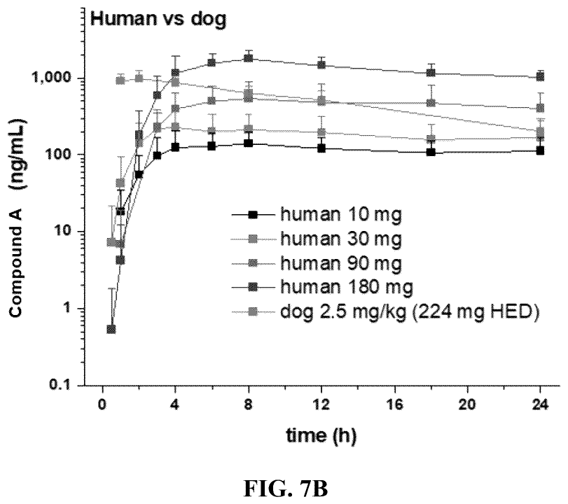

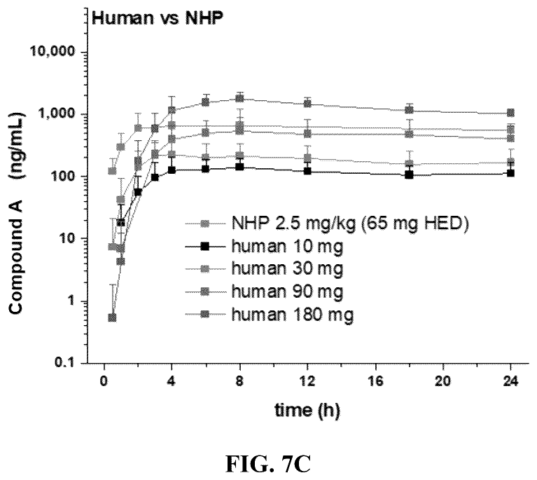

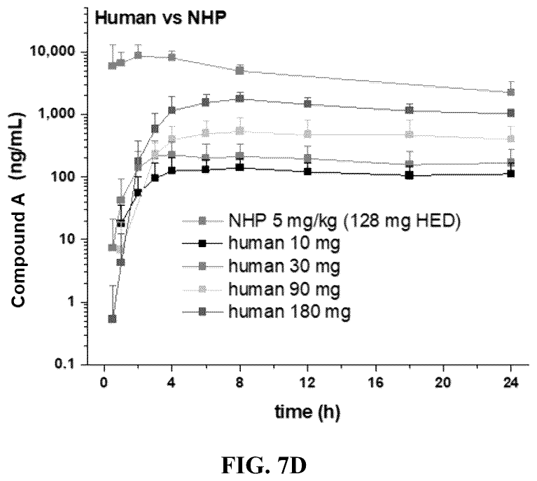

Protocol Summary (Study Design, Criteria for Evaluation), Footnotes c, d, f in Tables 2-4 and 2-5), Table 2-5, Figure 7-3, and Section 7.5 (End of Study Definition):

These sections were updated to define Day 180 (90 days post last dose) as the earliest possible End of Study visit in Parts 2 and 3. This time point was chosen based on timelines to suppress sperm counts to levels associated with male infertility in hormonal male contraceptive clinical trials and in men post vasectomy. Based on data from hormonal male contraceptive trials, it takes 6 months to reach azoospermia in 90% of study participants (Liu et al., 2008; J Clin Endocrinol Metab; DOI: 10.1210/jc.2007-2768). Vasectomy studies show that it takes 22 weeks on average to reach azoospermia in 82% of men after the procedure (Barone, et al., 2003; Journal of Urology; DOI: 10.1097/01.ju.0000075505.08215.28).

Protocol Summary (Study Design), Sections 7.1 (Overall Study Design and Plan), 7.1.2 (Multiple Ascending Dose), 7.1.3 (Cohort Expansion), 8.9 (Participants Replacement Criteria), 9.3.1 (Criteria for Dose Escalation) and 13.2 (Sample Size Determination), Figure 7-1, and Table 9-2:

Since dosing for 90 days is traditionally not considered a dosing period for a Phase 1 study, the definition of Part 2 was changed from Phase 1b to Phase 2a and the respective sections were updated accordingly.

### 2: Protocol Summary

Indication: Reduction of sperm count and sperm motility as a means for male contraception

### Objectives/Endpoints:

### Primary:

The primary objective is to assess the safety and tolerability of repeated daily oral dosing of Compound A for 28 or 90 days.

The primary endpoint is the incidence of Adverse Events (AEs), abnormal laboratory test results (clinical chemistry and liver function tests, CBC with differential [absolute and percentage], and lipid panel), ECGs (12-lead and Holter monitoring [Holter only in Part 1]), vital signs, and other safety assessments.

### Secondary:

The secondary objectives are to evaluate the pharmacokinetics (PK) and pharmacodynamics (PD) of Compound A.

The secondary endpoints are:

Pharmacodynamic semen parameters, including ejaculate volume, ejaculate pH, total sperm count per ejaculate, sperm concentration, and motility (total, progressive, non-progressive, immotile).

Plasma PK parameters, including area under the curve from time 0 extrapolated to infinity [AUCinf], area under the curve from time 0 to the last measured concentration [AUC0-t], area under the curve from time 0 to 24 hours [AUC0-24], time to maximum concentration [Tₘₐₓ], terminal elimination half-life, [T1/2], lag time [T_{lag}], apparent volume of distribution [Vz/F], oral clearance [CL/F], and maximum concentration [Cₘₐₓ] of Compound A.

Compound A concentrations in semen.

Pharmacodynamic parameters, including serum hormones (follicle-stimulating hormone [FSH], luteinizing hormone [LH], testosterone [T], estradiol [E2]), and sex hormone binding globulin (SHBG).

### Exploratory:

The exploratory objectives are:

To evaluate the effect of Compound A on change from baseline in corrected QT interval in ECG (QTc) following 28 or 90 oral doses in healthy male participants.

To evaluate the effect of 28-day or 90-day dosing with Compound A on inflammatory biomarkers.

The exploratory endpoints are:

Formal statistical analysis of relationship between the change from baseline in QT interval corrected for heart rate using Bazett's and Fridericia's formulation (AQTcF) and plasma Compound A con-centration.

Formal statistical analysis of AQTcF at each post-dose timepoint for the comparison between each active dose level.

Levels of inflammatory biomarkers, including TNF-alpha, IL-6, E-selectin, vWF, troponin and hsCRP.

Rationale: Gene targeting research has demonstrated that loss of function of retinoic acid receptors (RARs) inhibits spermatogenesis. Compound A is an orally administered RAR-alpha antagonist that reversibly inhibits spermatogenesis, spermiation, and sperm motility in animal models. In a single dose Phase 1a trial (NCT06094283), Compound A was safe and well tolerated at doses up to 180 mg. In the 28-day part of the present study (NCT06542237), Compound A has been safe and well tolerated at doses up to 90 mg. A dose of 180 mg for 28 days is currently being evaluated.

Although the safety data have shown that Compound A was well tolerated for 28 days, firm evidence of sperm reduction has not been observed, possibly because the duration of one spermatogenic cycle and epididymal transit in men is 85 days. Therefore, additional cohorts comprising 90-day administration have been added to the protocol as an amendment, in the same dose range as is being studied in the 28-day cohorts. These data will allow evaluation of the safety, tolerability, PK, and PD of Compound A in healthy human male adults. Data from this study will inform future program development.

Study Design: This is a Phase 1b/2a, open label, 28-day or 90-day repeat-dose escalation study of Compound A in healthy men to evaluate the safety, tolerability, PK and PD, and to assess sexual function and mood.

The study consists of 3 parts: a 28-day Phase 1b part (Part 1), a 90-day Phase 2a part (Part 2 [dose escalation]) and a 90-day Phase 2a part (Part 3 [cohort extension]). All participants will receive Compound A in the fasted state.

In Part 1, 4 dosing cohorts and one optional 5th cohort with 4 participants each will be evaluated. In Part 2, up to 4 dosing cohorts with 4 participants each will be evaluated. All dose levels will be at or below a dose that was deemed safe and well tolerated upon 28-day administration in Part 1. Participants in Parts 1 and 2 may be replaced automatically if prior to any dosing. If a participant is required to be replaced post-dose, the replacement may be approved at the discretion of Sponsor and PI with the objective of available data in at least 3 evaluable participants per cohort. In Part 3, up to 3 cohorts will receive doses within the range of doses that were well tolerated and had biological activity in Part 2. Each Part 3 cohort will have up to 10 participants. Participants who discontinue early may be replaced.

The study population in all parts of the study will include healthy men who have decided to have a vasectomy and are waiting for the procedure and men who, in the opinion of the investigator, have made a firm decision not to father children in the future. As an additional safety measure, a sperm sample from all participants will be collected before administering the investigational product. The sample will be cryopreserved at no cost before administering the investigational product until the last study visit (for participants who provide two successive sperm samples that fall into at least the 5th percentile of the WHO range of normality [WHO, 2010 and WHO, 2021] by the end of study visit), or for 10 years thereafter (for participants who do not provide two successive sperm samples that fall into at least the 5th percentile of WHO range of normality [WHO, 2010 and WHO, 2021] by the end of study visit). If a participant withdraws, his sperm sample will be destroyed if sperm parameters fall into at least the 5th percentile of the WHO range of normality (WHO, 2010 and WHO, 2021). If sperm parameters do not fall into at least the 5th percentile of the WHO range of normality (WHO, 2010 and WHO, 2021), the storage of the sperm sample will continue for the 10-year period, unless the participant requests the sample to be destroyed.

In Part 1, all participants will undergo preliminary screening procedures for the study (Day −28 to Day −2). Following successful enrollment, participants will be admitted in the morning of Day −1 (day before dosing), will receive study drug (Compound A) on Days 1-28, and will remain onsite until Day 7 (day of discharge). Participants will return to the clinic on Days 10, 14, 17, 21 and 24 for self-dosing under the supervision of clinic staff and have safety, PK and PD assessments performed. Participants will be readmitted to the clinic prior to dosing in the morning on Day 27 (the day before the last dosing) and remain onsite until 72 h post last dose (Day 31). On Days 1-7, participants will receive study drug (Compound A) under the supervision of clinic staff. On Days 7, 14 and 21, participants will receive a 7-Day supply of study drug (9 doses in total [7 regular, 2 extra]) for self-dosing. On Days 8, 9, 11, 12, 13, 15, 16, 18, 19, 20, 22, 23, 25 and 26, participants will self-dose at home under the supervision of a telehealth staff member via video call. On Days 10, 14, 17, 21 and 24, participants will return to the clinic for self-dosing under the supervision of clinic staff and have safety, PK and PD assessments performed.

In Parts 2 and 3, all participants will undergo preliminary screening procedures (up to Day −2). Following successful enrollment, participants will be admitted in the morning of Day −1 (day before dosing) and will remain onsite until Day 2 (day of discharge). Participants will be readmitted to the clinic in the evening of Day 89 and remain onsite until Day 91 (24 hours post last dose). Participants will return for PK assessments on Day 92 (48 hours post last dose) and Day 93 (72 hours post last dose). Participants will take the study drug (Compound A) on Days 1-90. On Day 2 (day of discharge), participants will receive a 12-day supply of study drug (14 doses in total [12 regular, 2 extra]) for self-dosing. On Days 14, 28, 42, 56, 70 and 84, they will return to the clinic to pick up a 14-day supply of study drug (16 doses in total [14 regular, 2 extra]) for self-dosing and have safety, PK and PD assessments performed. On Days 1, 2, 14, 28, 42, 56, 70, 84 and 90, participants will self-dose with study drug under the supervision of clinic staff. On Days 3-13, 15-27, 29-41, 43-55, 57-69, 71-83 and 85-89, participants will self-dose with no supervision. Participants are required to complete an electronic dosing diary daily on Days 1-90 to record when they self-dose.

In Part 1, follow-up telehealth visits (video call) will be performed on Days 35, 42 and 49 (+1 day) to ensure the ongoing wellbeing of all participants. In-person follow-up visits are planned on Day 42 (+1 day), and on Days 56, 84, 112, 140, 168, and 196 (+2 days) to assess PK and/or sperm parameters. Day 42 is the only day when participants will have a telehealth visit and an in-person visit to the fertility clinic for semen/sperm parameter assessments. In-person follow-up visits will be performed on Day 42 (+1 day), and on Days 56, 84 and 112 (+2 days) with participants whose sperm parameters on Days 28, 42 and 56 were at or above the 5th per-centile of the WHO range of normality (WHO, 2010 and WHO, 2021). For those participants, Day 112 will be the End of Study visit (approximately 1 spermatogenic cycle). In-person follow-up visits will be performed on Day 42 (+1 day), and on Days 56, 84, 112, 140, 168, and 196 (+2 days) with participants whose sperm parameters on Days 28, 42 and 56 were below the 5th percentile. For those participants, Day 196 will be the earliest End of Study visit. If sperm parameters have not returned to at least the 5th percentile of the WHO range of normality (WHO, 2010 and WHO, 2021) by Day 196 (approximately 2.5 spermatogenic cycles), sperm parameters will also be assessed until the resolution of any clinically relevant changes in their sperm parameters (as assessed at visits on Days 224, 252, and 280±2 days). The last possible End of Study visit will be Day 280±2 days.

After at least 3 evaluable participants in a cohort of Part 1 complete the 28-day dosing, safety data and available PK and PD data from up to Day 28 will be re-viewed by the Dose Escalation review Committee (DERC) to determine the dose level for the subsequent cohort.

In Parts 2 and 3, participants will be followed up for either 270 days after administration of the last dose (~4 spermatogenic cycles) or until two consecutive semen samples have sperm concentrations returned to baseline levels, whichever is earlier. In-person follow-up visits are planned on Day 98, 120, 150, 180, 210, 240, 270, 300, 330 and/or 360 (±2 days) to assess PK and/or sperm parameters. Day 180 will be the earliest possible End of Study visit.

All dose levels in Part 2 will be at or below a strength that was deemed safe and well tolerated upon 28-day administration in Part 1. After at least 3 evaluable participants of a Part 2 cohort complete the Day 42 dosing, safety data and available PK and PD data from up to Day 42 will be reviewed by the DERC.

After the review of the safety, and available PK and PD data from up to Day 90 of at least 3 evaluable participants from a Part 2 cohort with established safety and intended pharmacology (reduction of sperm concentration), Part 3 of this study will be initiated. All dose levels will be at a strength that was deemed safe and efficacious in Part 2.

Planned number of participants: In Part 1, 4 dosing cohorts and one optional 5th cohort with 4 participants will be evaluated (up to 20 participants). In Part 2, up to 4 dosing cohorts with 4 participants will be evaluated (up to 16 participants). Participants in Parts 1 and 2 may be replaced automatically if prior to any dosing. If a participant is required to be replaced post-dose, the replacement may be approved at discretion of Sponsor and PI to ensure available data in at least 3 evaluable participants per cohort. In Part 3, up to 3 cohorts of up to 10 participants (up to 30 participants) are planned. This number may increase as participants may be replaced. All participants will receive Compound A. Any replacement participants will receive the same dose as the participant they are replacing.

In Part 1, an evaluable participant is a study participant who has completed the planned safety assessments up to Day 28, and planned PK assessments up to 24 h after dosing on Day 1 and Day 28.

In Parts 2 and 3, an evaluable participant is a study participant who has completed the planned safety assessments up to Day 90, has at least 7 PK samples collected on Days 1 and 90 and has at least 4 evaluable semen samples collected starting on Day 28 during the dosing phase, and at least 4 evaluable semen samples collected starting on Day 120 during the recovery phase.

However, participants withdrawn due to an investigational medicinal product (IMP)-related AE will be considered as an evaluable participants and not be replaced.

### Test Product: Compound A Capsule

Dose, route, frequency: Once daily single oral dose for 28 days (Part 1) or 90 days (Parts 2 and 3). For Part 1, planned doses are 15 to 180 mg. For Part 2, planned doses are 30 to 180 mg. For Part 3, doses will be determined based on Part 2 results, but the highest Part 3 dose will not exceed the highest well tolerated Part 2 dose.

Treatment Regimes: All participants will fast (except water) for at least 10 hours overnight prior to administration of study drug (Compound A), and for at least 2 hours after dosing. Study drug (Compound A) will be administered with 240 mL of water. Water will be allowed ad libitum during the study, except for the time period between 1 hour before dosing and 1 hour after dosing, when the only water permitted will be the water administered with the study drug. Decaffeinated fluids will be allowed ad libitum from no earlier than 2 h post-dose. A late breakfast or morning snack should be taken no earlier than 2 h post-dose. Lunch should be taken at approximately 4 h post-dose, an evening meal at approximately 10 h post-dose, and an evening snack should be finished no later than 12 h post-dose.

In Part 1, study drug (Compound A) will be administered in the clinic by clinic staff and at home by self-dosing under supervision of telehealth staff via video call.

In Parts 2 and 3, study drug (Compound A) will be administered in the clinic by clinic staff and at home by self-dosing with no supervision. Participants will complete a dosing diary from Day 1-90.

In Part 1, the starting dose is 15 mg, with planned maximally 3-fold escalation (up to 180 mg). The DERC will utilize up to Day 28 safety, tolerability and available PK and PD data to determine the dose level for the subsequent cohort. In Part 2, the starting dose is 30 mg, with planned maximally 3-fold escalation (up to 180 mg). After at least 3 evaluable participants of a Part 2 cohort complete the Day 42 dosing, safety data and available PK and PD data from up to Day 42 will be reviewed by the DERC. To initiate Part 3, the DERC will utilize up to Day 90 safety, tolerability and available PK and PD data from a Part 2 cohort with established safety and intended pharmacology (reduction of sperm concentration). Subsequent dose levels will be selected based on safety, tolerability and available PK and PD data from Part 2 and any previous Part 3 cohort(s). In order to maximize the dose response information gained from the study, in either part, the next cohort may be given a dose that is lower than planned or that is between two prior doses or a different regimen.

### Criteria for Evaluation:

### Safety Parameters:

Each participant's health status will be monitored carefully throughout the study.

Baseline data collected at the screening visit will consist of the medical and andrological history, detailed physical examination (including height, weight, and BMI), ECG (12-lead and Holter monitoring [Holter only in Part 1]), vital signs, laboratory tests (including clinical chemistry, liver function, CBC with differential [absolute and percentage], hormones [serum T, FSH, LH, E2, and calculated free T], SHBG, HIV/hepatitis screening), urinalysis including toxicology, and semen analysis including ejaculate volume, ejaculate pH, total sperm count per ejaculate, sperm concentration, and motility (total, progressive, non-progressive, immotile).

Specific assessments to evaluate treatment safety include the following: the frequency and type of adverse events; safety laboratory tests (clinical chemistry and liver function tests), CBC with differential [absolute and percentage], and lipid panel; ECGs (12-lead and Holter monitoring [Holter only in Part 1]); and vital signs.

Changes in blood counts and clinical chemistry measures from baseline to recovery will be calculated for all participants in the treated population with available data. All of these data will be compared to mean changes from baseline and used to identify the incidence of clinically significant results indicative of safety concerns.

### Pharmacokinetics:

Part 1: Participants will have overnight stays in the clinical research unit in order to monitor their wellbeing and obtain 24-hour PK profiles on Day 1 and Day 28. For PK profiles, blood samples will be collected at −0.5 hours before (Day 1 only), and 0.5, 1, 2, 4, 6, 8, 12, 18 and 24 hours after oral dose administration.

Single time point blood samples will be collected prior to dosing on Days 3, 4, 7, 10, 14, 17, 21 and 24. Elimination will be assessed by measuring serum concentrations of Compound A at 48 and 72 hours (Days 30 and 31) and 28 days (Day 56) after the last dose.

Compound A concentrations in semen will be assessed once during screening, and on Days 21, 28 and 56. If Compound A is detectable in semen on Day 56, semen PK will also be analyzed on Day 84.

Parts 2 and 3: Participants will have overnight stays in the clinical research unit in order to monitor their well-being and obtain 24-hour PK profiles on Day 1 and Day 90. For PK profiles, blood samples will be collected at −0.5 hours before (Day 1 only), and 0.5, 1, 2, 4, 6, 8, 12, 18 and 24 hours after oral dose administration.

Single time point blood samples will be collected prior to dosing on Days 28, 56 and 84. Elimination will be assessed by measuring serum concentrations of Compound A at 48 and 72 hours (Days 92 and 93) and 30 days (Day 120) after the last dose.

Compound A concentrations in semen will be assessed once during Screening, and on Days 84, 120 and 150.

### Pharmacodynamics:

### Part 1:

Hormones (serum T, LH, FSH, E2, and calculated free T), SHBG and inflammatory biomarkers (TNF-alpha, IL-6, E-selectin, vWF and hsCRP) will be measured once during screening, at −0.5 hours pre-dose (Day 1), on Days 3, 7, 14, 21, 28, 56 and 112 (earliest End of Study Visit), Days 196, 224 or 252 (possible End of Study visits) or on Day 280 (latest End of Study visit).

### Parts 2 and 3:

Semen samples will be collected three times during Screening, and once on Days 28, 42, 56, 70, 84, 98, 120, 150, 180 (earliest possible End of Study visit), 210, 240, 270, 300, 330 and/or 360 (latest End of Study visit).

Hormones (serum T, LH, FSH, E2, and calculated free T), SHBG and inflammatory biomarkers (TNF-alpha, IL-6, E-selectin, vWF, troponin, hsCRP) will be measured once during Screening, at −0.5 hours pre-dose (Day 1), on Days 14, 28, 42, 56, 70, 84, 90, 120, 150, 180 (earliest possible End of Study visit), 210, 240, 270, 300, 330 and/or 360 (latest End of Study visit).

Statistical method: The following analysis populations are planned for this study:

Screening Population: all participants who provide informed consent and demo-graphic and/or baseline Screening assessments.

Safety Population: all participants who receive any amount of study drug.

Per Protocol Population: all participants who receive at least 80% of the planned doses of study drug.

Pharmacokinetic Population in Part 1: all participants who receive study drug and have at least 7 PK samples collected on Days 1 and 28.

Pharmacokinetic Population in Parts 2 and 3: all participants who receive study drug and have at least 7 PK samples collected on Days 1 and 90.

Pharmacodynamic Population in Part 1: all participants who receive study drug and have at least 2 evaluable semen samples collected starting on Day 21 or later during the study.

Pharmacodynamic Population in Part 2 and 3: all participants who receive study drug and have at least 4 evaluable semen samples collected starting on Day 28 during the dosing phase, and at least 4 evaluable semen samples collected starting on Day 120 during the recovery phase.

### Participant Characteristics and Disposition:

The number of participants completing the study, and withdrawing from the study, along with reasons for withdrawal, will be tabulated overall and by treatment group. The number of participants in each analysis population will be reported. Demographic data will be summarized and include age, height, weight, race, ethnicity, prior and concomitant medications, and medical and andrological history.

### Safety Analyses:

Safety data, including AEs, safety laboratory tests (clinical chemistry and liver function tests, CBC with differential [absolute and percentage], and lipid panel), vital signs, ECGs, and physical examinations, will be summarized. Changes in blood counts, coagulation, and clinical chemistry measures from baseline to recovery will be calculated for all participants.

### Clinical Pharmacology Analyses:

Non-compartmental methods will be used to calculate PK parameters for Com-pound A where appropriate. PK profiles and parameters collected over 24 hours on Days 1 and 28 (Part 1), and on Days 1 and 90 (Parts 2 and 3) will be evaluated and summarized.

Dose proportionality will be evaluated using a power model for log transformed PK parameters Cₘₐₓ, AUC0-t, and AUCinf including criteria defined by Hummel et al. (Hummel et al., 2009).

Pharmacodynamic variables will be summarized using descriptive statistics including changes from baseline.

Sample size determination: No formal sample size calculations were performed. The cohort size was estimated based on Shen et al. (Shen et al., 2019). For Part 1, it is planned to enroll 16 healthy male participants in total (4 cohorts of 4 participants) or 20 participants if the optional 5th cohort is recruited. For Part 2, it is planned to enroll up to 16 healthy male participants in total (up to 4 cohorts of 4 participants). For Part 3, it is planned to enroll up to 30 healthy male participants in total (3 cohorts of up to 10 participants). As many participants per Part 1 and Part 2 cohort may be replaced as needed to ensure a minimum of 3 evaluable participants per cohort. Participants who discontinue from Part 3 may or may not be replaced.

Study and treatment duration: The participants for Part 1 of the study will be evaluated for approximately 24 to 44 weeks (screening up to 4 weeks, dosing 4 weeks, and post-dosing between 16 and 36 weeks). The participants for Parts 2 and 3 of the study will be evaluated for approximately up to 56 weeks (screening at least 4 weeks, dosing 13 weeks, and recovery up to 39 weeks).

### Phase 1b MAD Study (Part 1)

The safety, tolerability, PK, and PD of multiple ascending oral doses of Compound A are currently under study in this open label dose escalation study in healthy men (ID NCT06542237). The following section summarizes the results that have been collected so far from Part 1 (28-day dosing): A total of 16 participants in 4 cohorts of 4 participants were enrolled to receive 15, 30 or 90 of study drug (Compound A) under fasting conditions once daily for 28 days. Cohort 4 (receiving 180 mg) is currently being studied.

To date, no participant withdrawal has occurred, no study stopping criteria have been reached, and no serious adverse events (SAEs) have been reported. Five AEs were classified as possibly related to study treatment: moderate ventricular triplet, mild skin rash, mild sunburn, mild increased urinary frequency and mild photosensitivity. All AEs (except for photosensitivity) were temporary, self-limiting, and resolved without sequelae.

Based on the review of the safety data (clinical chemistry, hematology, urinalysis, vital signs, ECG [12-lead and Holter monitoring], physical examination and AEs), Compound A at a strength of up to 90 mg in the fasted state was deemed safe and well tolerated upon 28-day oral administration under the conditions of this trial. Available key PK parameters are summarized in TABLE 11. TABLE 12 compares the available exposure data (Cₘₐₓ and AUC) to the doses tested so far.

TABLE 11
Key PK parameters of Compound A in healthy volunteers following
28 days of oral dose administration of Compound A

| | | Tₘₐₓ | Cₘₐₓ | AUC₀₋₂₄ | AUC₀₋ₗₐₛₜ | AUC_{0-inf} | T_{1/2} |
|---|---|---|---|---|---|---|---|
| | | (h) | (ng/mL) | (h*ng/mL) | (h*ng/mL) | (h*ng/mL) | (h) |
| | Day | Mean ± SD | | | | | |
| 15 mg | Day 1 | 5.00* | 274.8 | 4,851 | 4,851 | N/A | N/A |
| | | (4.00-6.00) | (99.5) | (1,639) | (1,639) | | |
| | Day | 5.00* | 348.3 | 6,416 | 24,690 | 27,510 | 35.8 |
| | 28 | (1.00-6.00) | (35.7) | (851.2) | (11,080) | (11,860) | (8.9) |
| 30 mg | Day 1 | 4.00* | 544.5 | 10,060 | 10,060 | N/A | N/A |
| | | (4.00-12.0) | (105.2) | (1,432) | (1,432) | | |
| | Day | 6.00* | 551.5 | 9,585 | 36,120 | 36,400 | 28.9 |
| | 28 | (2.00-24.0) | (240.6) | (4,264) | (8,437) | (10,320) | (1.2) |
| 90 mg | Day 1 | 6.00* | 794.3 | 14,090 | 14,090 | N/A | N/A |
| | | (4.00-12.00) | (395.7) | (7,026) | (7,026) | | |
| | Day | 5.00* | 1,165 | 22,710 | N/A | N/A | N/A |
| | 28 | (4.00-12.00) | (211.4) | (4,730) | | | |

*Shown is median (range)
N/A—not available yet

TABLE 12
Dose proportionality of Compound A in healthy volunteers
following 28 days of oral dose administration

| Compared | | | Cₘₐₓ | | AUC₀₋₂₄ | |
|---|---|---|---|---|---|---|
| doses | Dose | | values | Cₘₐₓ | values | AUC₀₋₂₄ |
| (mg/kg/day) | Ratio | Day | (ng/mL) | Ratio | (h*ng/mL) | Ratio |
| 30 over 15 | 2.0 | Day 1 | 544.5/ | 2.0 | 10,060/ | 2.1 |
| | | | 274.8 | | 4,851 | |
| | | Day 28 | 551.5/ | 1.6 | 9,585/ | 1.5 |
| | | | 348.3 | | 6,416 | |
| 90 over 30 | 3.0 | Day 1 | 794.3/ | 1.5 | 14,091/ | 1.4 |
| | | | 544.5 | | 10,060 | |
| | | Day 28 | 1,165/ | 2.1 | 22,710/ | 2.4 |
| | | | 551.5 | | 9,585 | |
| 90 over 15 | 6.0 | Day 1 | 794.3/ | 3.2 | 14,091/ | 2.9 |
| | | | 247.8 | | 4,851 | |
| | | Day 28 | 1,165/ | 3.3 | 22,710/ | 3.5 |
| | | | 348.3 | | 6,414 | |

### Summary of Potential Risks and Benefits

This amended three-part Phase 1b/2a study represents the first time Compound A will be administered repeatedly for up to 90 days to healthy male volunteers. Research in mice, rats, dogs and NHPs suggests that Compound A does not alter testosterone levels, and that it does not affect virility. This has also been confirmed in the 28 enrolled male participants who received Compound A. The impairment of fertility in nonclinical models was shown only after repeat dosing for 3-4 weeks and was reversible after treatment discontinuation in all four species: mouse, rat, dog and NHP. Dosing men for 4 weeks with Compound A at doses of up to 90 mg was not sufficient to decrease sperm counts. The main underlying reason provided by experts in the male contraceptive field is the dosing duration. Dosing for 28 days in men only covers approximately one third of the entire cycle from sperm production to sperm release, which takes 85 days in men (71 days for one spermatogenic cycle and 14 days for epididymal transit). Clinical trials with hormonal methods demonstrate that it takes a median time of 2.73 months to suppress sperm concentrations below fertile limits (Liu et al., 2008). Therefore, to study the effect of longer dosing on the intended effect (reduction in sperm concentration), the dosing period will be extended from 28 days to 90 days.

Out of precaution, only men who have decided to have a vasectomy and are waiting for the procedure, or men who, in the opinion of the investigator, have made a firm decision not to father children in the future will be enrolled in this Phase 1b/2a clinical trial. As an additional safety measure, a sperm sample from all participants will be collected and cryopreserved at no cost before administering the investigational product until the last study visit (for participants who provide two successive sperm samples that fall into at least the 5th percentile of the WHO range of normality [WHO, 2010 and WHO, 2021] by the end of study visit), or for 10 years thereafter (for participants who do not provide two successive sperm samples that fall into at least the 5th percentile of WHO range of normality [WHO, 2010 and WHO, 2021] by the end of study visit), unless the participant requests the sample to be destroyed. If a participant withdraws, his sperm sample will be destroyed if sperm parameters fall into at least the 5th percentile of the WHO range of normality (WHO, 2010 and WHO, 2021). If sperm parameters do not fall into at least the 5th percentile of the WHO range of normality (WHO, 2010 and WHO, 2021), the storage of the sperm sample will continue for the 10-year period, unless the participant requests the sample to be destroyed.

Repeat daily dosing of ≥125 mg/kg/day in the rat and ≥10 mg/kg/day in the dog were not tolerated resulting in death and morbidity. These doses convert to respective HEDs of ≥1,612.8 mg and ≥444.8 mg fixed dose based on 80 kg body weight. In the rat at non-tolerated doses, the hematopoietic, hepatic, adrenal and lymphoid systems were affected. In the dog, the cause of death and moribund condition was attributed to renal toxicity with additional pathological changes in multiple organ systems considered potentially secondary to the renal changes. While such changes are not expected with repeated dosing at the proposed dose ranges (15-180 mg), standard safety monitoring measures are part of the study and include monitoring liver toxicity (through regular laboratory analyses), renal toxicity (through regular laboratory analyses, physical exams and urinalyses), cardiac toxicity (through ECGs, laboratory analyses and physical exams) and endocrine toxicity (through laboratory analyses and physical exams). Safety laboratory analyses include serum chemistry, hematology, coagulation, and lipid panel.

Compound A has no genotoxic potential based on findings from an in vitro bacterial reverse mutation (Ames) test, a chromosomal aberration assay, and an in vivo rat micronucleus study. Direct in vitro inhibition of CYP1A2, CYP2B6, CYP2C8, CYP2C9, CYP2C19, CYP2D6 and CYP3A4/5 (midazolam and testosterone as substrates) was observed for Compound A with respective IC50 values of 30.1, 6.10, 8.37, 14.6, 85.1, >100, 58.6, and 53.6 μM. Compound A had NADPH-independent time-dependent inhibition against CYP2D6 and CYP3A4/5, and no TDI potential against CYP1A2, CYP2B6, CYP2C8, CYP2C9, and CYP2C19 over a concentration range of 0.3 to 100 μM. However, the likelihood of drug interactions is low since healthy participants who meet eligibility criteria will be enrolled in this Phase 1b/2a clinical trial. An allowance for select concomitant medications may be considered for select participants at the discretion of the investigator and/or sponsor if drug profiles are not anticipated to interact with Compound A. Such medications may include but will not be limited to paracetamol or ibuprofen as assessed by both the PI and the Sponsor. Exceptions may apply, as determined by the investigator, if each of the following criteria are met: medication with a short half-life if the washout is such that no PD activity is expected by the time of dosing with IMP; and if the use of medication does not jeopardize the safety of the trial participant; and if the use of medication is not considered to interfere with the objectives of the study. Additional precautions to avoid grapefruit, pomegranates, star fruit, or Seville oranges for 7 days prior to admission, and juices and products containing these fruits for the duration of the study will be taken.

Based on results from an in vitro molar extinction coefficient assay, Compound A has the potential to cause phototoxicity from UV light and further testing for potential phototoxicity is needed. As a result, participants will be advised to minimize exposure to sunlight, wear sunglasses and clothing that minimizes exposure to sunlight, and use sun cream on exposed areas when going outdoors during the 28-Day dosing period and until 10 days post last dose.

Collecting a blood sample from a vein may cause pain, swelling, bruising, light-headedness, fainting, and very rarely, clot formation, nerve damage and/or infection at the site of the needle stick.

During cannulation, more than one attempt may be needed to insert the cannula in a vein of a participant, and it is possible that bruising and/or inflammation may be experienced at the site of cannulation.

Electrocardiogram (ECG) stickers on the participants' chests and limbs may cause some local irritation and may be uncomfortable to remove but participants will be closely monitored to ensure any local irritation does not persist.

### IMP Related Risk

Against the background of the Coronavirus Disease 2019 (COVID-19) pandemic, the potential risk of a participant developing COVID-19 has been considered in terms of the risk-benefit evaluation. The mode of action of the IMP has been considered alongside available pre-clinical and clinical data (including class effects) and it is considered that a participant would not be at increased risk of either becoming infected with severe acute respiratory syndrome coronavirus 2 (SARS-COV-2; the virus that causes COVID-19) or experiencing a more severe illness. That is, the IMP has no known immunomodulatory effect that would confer an increased risk to healthy participants enrolled in the study.

### Objectives

### Primary Objective

The primary objective is to assess the safety and tolerability of repeated daily oral dosing of Compound A for 28 or 90 days.

### Secondary Objectives

The secondary objectives are:

To evaluate the pharmacodynamics (PD) of Compound A.

To assess the pharmacokinetics (PK) of Compound A.

### Exploratory Objectives

The exploratory objectives are:

To evaluate the effect of Compound A on change from baseline corrected QT interval in ECG (QTc) interval.

To evaluate the effect of Compound A on inflammatory biomarkers.

### Study Design

### Overall Study Design and Plan

Study Compound A-02 is a Phase 1b/2a, open label, dose escalation study to evaluate the safety, tolerability, PK, and PD of repeated daily dosing of Compound A for 28 or 90 days.

The study consists of 3 parts: a 28-day Phase 1b part (Part 1), a 90-day Phase 2a part (Part 2 [dose escalation]) and a 90-day Phase 2a part (Part 3 [cohort extension]).

In Part 1, 4 dosing cohorts and one optional 5th cohort with 4 participants each will be evaluated. All participants will receive Compound A in the fasted state. In Part 2, up to 4 dosing cohorts with 4 participants each will be evaluated. All dose levels will be at or below a strength that was deemed safe and well tolerated upon 28-day administration in Part 1. Participants may be replaced automatically if prior to any dosing. If a participant is required to be replaced post-dose, the replacement may be approved at discretion of Sponsor and PI to ensure available data in at least 3 evaluable participants per cohort. In Part 3, up to 3 cohorts with up to 10 participants will receive doses within the range of doses that were well tolerated and had biological activity in Part 2. Participants who discontinue early may be replaced. All participants in Parts 2 and 3 will receive Compound A in the fasted state.

### Multiple Ascending Dose (Phase 1b [Part 1])

In Part 1 of the study, 4 dosing cohorts and one optional 5th cohort with 4 participants (up to 20 participants) will be evaluated (TABLE 13). The starting dose for this study is 15 mg, with planned maximally 3-fold escalation (up to 180 mg), which will be based on emerging safety, PK and PD data. All participants will receive study drug under fasting conditions.

TABLE 13

| | | | Route of |
|---|---|---|---|
| Cohort | IMP | Planned Dose | Administration |
| 1 | Compound A | 15 mg | Oral, Fasted |
| 2 | Compound A | 30 mg | Oral, Fasted |
| 3 | Compound A | 90 mg | Oral, Fasted |
| 4 | Compound A | 180 mg | Oral, Fasted |
| 5 (optional) | Compound A | TBD^{a} | Oral, Fasted |

^{a}The selected dose will depend on emerging safety, PK and PD data from the previous cohort.
Abbreviations: TBD = to be determined.

The starting dose for Compound A in the multiple ascending dose (MAD) Part 1 is 15 mg. Participants will fast (except water) for at least 10 hours overnight prior to administration of study drug (Compound A), and for at least 2 hours after dosing.

Laboratory data will be examined on an ongoing basis by the Principal Investigator and/or Medical Monitor for any signs of drug induced liver injury (DILI).

The DERC will evaluate safety, tolerability and available PK and PD data from at least 3 evaluable participants from up to Day 28 to determine the dose level for the subsequent cohort.

The specific scope, roles, membership, decision making time points, and decision-making processes of the DERC will be outlined in the DERC Charter. All decisions to repeat a dose level, proceed to the next dose level or de-escalate the dose will be properly recorded in a source document. These decisions will be made in agreement with the Sponsor.

### Multiple Ascending Dose (Phase 2a [Part 2])

In Part 2 of the study, up to 4 dosing cohorts with 4 participants (up to 16 participants) will be evaluated (TABLE 14). The starting dose for this study is 30 mg, with planned maximally 3-fold escalation (up to 180 mg), which will be based on emerging safety, PK and PD data. All participants will receive study drug under fasting conditions.

TABLE 14

| | | | Route of |
|---|---|---|---|
| Cohort | IMP | Planned Dose | Administration |
| 1 | Compound A | 30 mg | Oral, Fasted |
| 2 | Compound A | TBD^{a} | Oral, Fasted |
| 3 | Compound A | TBD^{a} | Oral, Fasted |
| 4 | Compound A | TBD^{a} | Oral, Fasted |

^{a}The selected dose will depend on emerging safety, PK and PD data from the previous cohort.
Abbreviations: TBD = to be determined.

The starting dose for Compound A in the MAD Part 2 is 30 mg. Laboratory data will be examined on an ongoing basis by the Principal Investigator and/or Medical Monitor for any signs of DILI.

The DERC will evaluate safety, tolerability and available PK and PD data from at least 3 evaluable participants from up to Day 42 to determine the dose level for the subsequent cohort.

The specific scope, roles, membership, decision making time points, and decision-making processes of the DERC will be outlined in the DERC Charter. All decisions to repeat a dose level, proceed to the next dose level or de-escalate the dose will be properly recorded in a source document. These decisions will be made in agreement with the Sponsor.

### Cohort Expansion (Phase 2a [Part 3])

In Part 3, up to 3 cohorts of up to 10 participants (up to 30 participants) will receive doses within the range of doses that were well tolerated and had biological activity in Part 2.

Cohort expansion will be initiated after the review of the safety, and available PK and PD data from up to Day 90 of at least 3 evaluable participants of a Part 2 cohort with established safety and biological activity. Dose levels of subsequent Part 3 cohorts will be selected based on emerging data from Part 2 and previous Part 3 cohorts. All participants will receive study drug under fasting conditions.

An overview of the study design is provided in TABLE 15. TABLE 16 summarizes the study sequence for Part 1. TABLE 17 summarizes the study sequence for Parts 2 and 3.

TABLE 15
Study Design for Parts 1 and 2 (Dose Escalation)
and Part 3 (Cohort Expansion)

| | |
|---|---|
| Part | Cohort 1-1b - Dose Level 1: |
| 1 | 4 Active |
| (Dose | 28-Day dosing |
| Escalation) | DERC ↓ |
| | Cohort 2-1b - Dose Level 2: |
| | 4 Active |
| | 28-Day dosing |
| | ↓ DERC |
| | Cohort 3-1b - Dose Level 3: |
| | 4 Active |
| | 28-Day dosing |
| | ↓ DERC |
| | Cohort 4-1b - Dose Level 4: |
| | 4 Active |
| | 28-Day dosing |
| | ↓ DERC |
| Part | Cohort 5-2a - Dose Level 1: |
| 2 | 4 Active |
| (Dose | 90-day dosing |
| Escalation) | DERC ↓ |
| | Cohort 6-2a - Dose Level 2: |
| | 4 Active |
| | 90-day dosing |
| | ↓ DERC |
| | Cohort 7-2a - Dose Level 3: |
| | 4 Active |
| | 90-day dosing |
| | ↓ DERC |
| | Cohort 8-2a - Dose Level 4: |
| | 4 Active |
| | 90-day dosing |
| | ↓ DERC |
| Part | Cohort 9-2a - Dose Level 1: |
| 3 | Up to 10 Active |
| (Cohort | 90-day dosing |
| Expansion) | ↓ DERC |
| | Cohort 10-2a - Dose Level 2: |
| | Up to 10 Active |
| | 90-day dosing |
| | ↓ DERC |
| | Cohort 11-2a - Dose Level 3: |
| | Up to 10 Active |
| | 90-day dosing |

TABLE 16
Screening
Day −28 to Day −2
↓
Admission
(Day −1)
↓
Dosing
Days 1-28
↓
Discharge
Day 7
↓

| Follow-up | Follow-up |
|---|---|
| Visits | Visits |
| (Telehealth | (In-Person) |
| Video Call) | Days 10, 14, |
| Days 8, 9, 11, | 17, 21, 24 |
| 12, 13, 15, 16, | |
| 18, 19, 20, 22, | |
| 23, 25, 26 | |

↓
Admission
Day 27
↓
Discharge
Day 31
Follow-up Visits (Telehealth
Video Call)
Days 35, 42 ^{a}, 49
↓
Follow-up Visits (In Person)
Days 42 ^{a}, 56, 84, 112 ^{b}, 140, 168,
196 ^{c}, 224 ^{c}, 252 ^{c}, 280 ^{d}
^{a} Including a visit to the Fertility Clinic
^{b} Earliest End of Study visit (based on sperm parameters).
^{c} Possible End of Study visit (based on sperm parameters).
^{d} Latest End of Study visit.

TABLE 17

Screening
up to Day −2
↓
Admission
Day −1 (morning)
↓
Dosing
Days 1-90
↓
Discharge
Day 2
↓
Follow-up Visits
(In-Person)
Days 14, 28, 42, 56, 70, 84
↓
Admission
Day 89 (evening)
↓
Discharge
Day 91
↓
Follow-up Visits (In Person)
Days 92, 93, 98 ^{a}, 110, 120, 150,
180 ^{b}, 210, 240, 270, 300, 330,
360 ^{c}

^{a} Visit to the Fertility Clinic only
^{b} Earliest possible End of Study visit (based on sperm parameters)
^{c} Latest End of Study visit.

### Rationale and Discussion of Study Design

This is the first human repeat dose study of Compound A, a small molecule being developed as a potential male non hormonal contraceptive. In preclinical studies, no impact on sperm parameters was observed with single doses of Compound A as high as 1,000 mg/kg in rats (HED 161.29 mg/kg). The pharmacological on target effect-reduction of sperm count—of Compound A in nonclinical models was only shown after repeat dosing for 3-4 weeks. So far, these results have not been replicated in Part 1 of this ongoing clinical study upon administration of 28 oral doses up to 90 mg of Compound A to healthy male participants. The main underlying reason provided by experts in the male contraceptive field is the dosing duration. Dosing for 28 days in men only covers approximately one third of the entire cycle from sperm production to sperm release, which takes 85 days in men (71 days for one spermatogenic cycle and 14 days for epididymal transit). Clinical trials with hormonal methods demonstrate that it takes a median time of 2.73 months to suppress sperm concentrations below fertile limits (Liu et al., 2008). Therefore, the dosing period will be extended from 28 days to 90 days with the anticipation that this will show the intended effect (reduction in sperm concentration).

Out of precaution, only men who have decided to have a vasectomy and are waiting for the procedure, or men who, in the opinion of the investigator, have made a firm decision not to father children in the future, will be selected as the study population. As an additional safety measure, a sperm sample from all participants will be collected and cryopreserved at no cost before administering the investigational product until the last study visit (for participants who provide two successive sperm samples that fall into at least the 5th percentile of the WHO range of normality [WHO, 2010 and WHO, 2021] by the end of study visit), or for 10 years thereafter (for participants who do not provide two successive sperm samples that fall into at least the 5th percentile of WHO range of normality [WHO, 2010 and WHO, 2021] by the end of study visit). If a participant withdraws, his sperm sample will be destroyed if sperm parameters fall into at least the 5th percentile of the WHO range of normality (WHO, 2010 and WHO, 2021). If sperm parameters do not fall into at least the 5th percentile of the WHO range of normality (WHO, 2010 and WHO, 2021), the storage of the sperm sample will continue for the 10-year period, unless the participant requests the sample to be destroyed.

Safety data, and PD and PK data (where available) will be evaluated before proceeding to the next dose level.

Vital signs, ECGs (12-lead and Holter monitoring), and clinical laboratory tests will be performed to monitor the well-being of participants. Participants will also be closely monitored for any signs of liver, endocrine, cardiac or kidney impairment as well as changes in blood parameters.

Pharmacodynamic endpoints have been selected to evaluate effects on spermatogenesis, spermiation, and sperm motility. In Part 1, semen samples will be collected from all participants 3 times during the screening period, on Days 21 and 28 during the dosing period, and on Days 42, 56, 84, 112, 140, 168 and 196. If sperm parameters have not returned to at least the 5th percentile of WHO ranges of normality (WHO, 2010 and WHO, 2021; ≥15 million sperm cells/mL, ≥39 million/sperm cells in total ejaculate, ≥40% total motility, ≥30% progressive motility) by Day 196 (possible End of Study visit; approximately 2.5 spermatogenic cycles), semen samples will also be collected until the resolution of any clinically relevant changes in their sperm parameters (as assessed at visits on Days 224, 252, and 280±2 days). The last possible End of Study visit will be Day 280±2 days. In Parts 2 and 3, semen samples will be collected from all participants 3 times during the screening period and on Days 28, 42, 56, 70 and 84 during the dosing period. Participants will be followed up for either 270 days after administration of the last dose (~4 spermatogenic cycles) or until two consecutive semen samples have sperm concentrations at baseline levels, whichever is earlier. In-person follow-up visits are planned on Days 98, 120, 150, 180, 210, 240, 270, 300, 330 and/or 360 (+2 days). Concentrations of Compound A will also be assessed in semen samples. Blood samples for PK assessments will be collected throughout the study.

The study design, protocol procedures, schedule of assessments and decision-making procedures are aimed to protect the participants' safety and generate quality data that will inform the potential future clinical development of Compound A.

### Selection of Doses in the Study

The proposed starting dose for Compound A in the multiple ascending dose Part 1 of this study is 15 mg. This dose has been selected based on safety and tolerability data from the first in human Phase 1a study where single doses of Compound A from 10 mg to 180 mg were found to be safe and well tolerated.

Based on a body weight of 80 kg, the proposed starting dose of 15 mg is 0.6-fold above the HED of the MABEL in the rat, 7.4-fold below the HED of the MABEL in the dog and 4.3-fold below the HED of the MABEL in the NHP.

The proposed dose escalation for Compound A in Part 1 part of the study is as follows:
Cohort 1:15 mg Compound A
Cohort 2:30 mg Compound A
Cohort 3: The selected dose will depend on emerging safety, PK and PD data from Cohort 2.
Cohort 4: The selected dose will depend on emerging safety, PK and PD data from Cohort 3.

Dose escalation between dose levels will be maximally 3-fold.

The proposed starting dose for Compound A in Part 2 of this study is 30 mg. This dose has been selected based on safety and tolerability data from Part 1 of this study where 28-day oral administration was found to be safe and well tolerated.

At this stage of development, it is still difficult to predict the range of doses and the dose regimens that could provide optimal efficacy, reversibility, and adequate tolerability. Considering the non-GLP NHP repeat dose study findings as the closest model to human biology, a potential efficacious dose could range from 2.5 to 7.5 mg/kg/day (equivalent to HEDs ranging from 0.81 to 2.42 mg/kg/day) on repeat dose administration. Considering that the average weight for male adults in Oceania is around 70-80 kg (Walpole et al., 2012), this could translate to doses between 57-170 mg/day (70 kg BW) and 65-194 mg/day (80 kg BW), which covers the range of doses planned to be explored in Part 1 (28-day repeated dose escalation study with 15-180 mg) and in Part 2 and 3 (90-day repeated dose escalation study with proposed doses of 30-180 mg). As more information is gathered on the safety, PK and PD of Compound A it will be reasonable to consider and explore if higher doses given less frequently could also result in similar safety and efficacy with added convenience compared to lower daily doses.

### End of Study Definition

The clinical study will be considered completed when the final participant's last follow-up visit has occurred. In Part 1, this is either Day 112 [for participants whose sperm parameters on Days 28, 42 and 56 were at or above the 5th percentile of the WHO range of normality (WHO, 2010 and WHO, 2021)] or between Days 196 and 280 [for participants whose sperm parameters on Days 28, 42 and 56 were below the 5th percentile of the WHO range of normality (WHO, 2010 and WHO, 2021)]). In Parts 2 and 3, participants will be followed up for either 270 days after administration of the last dose (~4 spermatogenic cycles) or until two consecutive semen samples have sperm concentrations at baseline levels, whichever is earlier. The earliest possible End of Study visit is Day 180 (+2 days). The latest End of Study visit is Day 360 (+2 days). If the study is terminated prematurely, the study ends when the sponsor notifies the investigator in writing that the trial has ended or when the last participant completes the final follow-up, whichever is later. Any changes to this definition will be notified as a substantial amendment.

Medsafe and Therapeutic Goods Administration (TGA) will be notified of the conclusion of the study within 90 days of the end of the study, or within 15 days if the study is terminated early, clearly explaining the reasons for the termination.

### Treatments

Compound A will be administered orally in Swedish orange size 0 hard gelatin capsules.

TABLE 18

| | | Route of |
|---|---|---|
| IMP Name | Planned Dose | Administration |
| Compound A Capsule, | 15 to 180 mg | Oral, Fasted |
| 15-100 mg | | |

The Compound A capsule is an investigational product for use only in the proposed clinical trial. Only participants enrolled in the study may receive study treatment and only authorized site staff may supply or administer study treatment. Quotient Sciences is manufacturing the IMP, and the suitability of the manufacturing process will be documented in a Pharmaceutical Development and Control Strategy Report. IMP will be reconciled and destroyed in accordance with the study-specific plan.

### Selection of Timing of Dose for Each Participant

Dosing of participants will start on Day 1. Study drug (Compound A) will be administered after an overnight fast of at least 10 hours with 240 mL of water. Participants should also fast for at least 2 hours after dosing.

Water will be allowed ad libitum during the study, except for the time period between 1 hour before dosing and 1 hour after dosing, when the only water permitted will be the water administered with the study drug. Decaffeinated fluids will be allowed ad libitum from no earlier than 2 h post-dose.

A late breakfast or morning snack should be taken no earlier than 2 h post-dose. Lunch should be taken at approximately 4 h post-dose, an evening meal at approximately 10 h post-dose, and an evening snack should be finished no later than 12 h post-dose.

### Dose Adjustment Criteria

Safety, PK, and PD data of up to Day 28 (Part 1) or Day 42 (Parts 2 and 3), where applicable, will be evaluated for dosing decisions for:

Dose selection for the next dose level.

Changes to safety and/or PK time points, if there is reason to believe that the change might im-prove the quality of the data or the safety of the participants as a consequence of review of emerging data.

### Criteria for Dose Escalation (Phase 1b [Part 1] and Phase 2a [Part 2])

Progression to the next dose level will be permitted if the review of safety, PK and sperm PD data (where available) of up to Day 28 (Part 1) or Day 42 (Part 2) in at least 3 evaluable participants suggests that it is safe to do so. Sperm PD data will be reviewed for efficacy (reduction of sperm count and/or sperm motility as the anticipated pharmacological effect).

Data provided by the clinical research units will be provided to the DERC in accordance with their respective standard operating procedure (SOP) on interim dose decision making and dose escalation. The decision will be documented and signed by the investigator as per clinical re-search unit's current SOP. Evidence of the decision will be retained in the Investigator Site File (ISF).

For dose escalation to proceed, data from up to Day 28 (Part 1) or Day 42 (Part 2) must be available from a minimum of 3 evaluable participants who have completed the planned safety assessments, and planned PK and PD assessments (where available). Dose increases will only be made after a complete review of all data collected from the previous dose group by the DERC. The following data are required:
AEs (mandatory)
Concomitant medications (if used)
Vital signs
Clinical laboratory results, including serum hormones
ECGs
Physical examinations
Plasma concentrations of Compound A
Interim PK parameter estimations (Cmax, Tmax, AUC)
Interim blood PD parameters estimations
Semen and sperm parameter data
Rules for dose decision are as follows:
Dose escalation between MAD periods will be a maximum 3-fold.

Sperm PD data will only be reviewed for efficacy (reduction of sperm count and/or sperm motility as the anticipated pharmacological effect).

Part 1: If ≥75% of participants within a cohort- or the lowest numerical approximation of the calculated percentage-experience a clinically relevant decrease from a normal baseline in sperm parameters on Day 28 that fall below the 2.5th percentile of WHO range of normality (WHO, 2010 and WHO, 2021), the dose will be escalated to the next planned dose level.
<9 million sperm cells/mL
<23 million/sperm cells in total ejaculate
<34% total motility
<24% progressive motility

Parts 2 and 3: If >75% of participants within a cohort- or the lowest numerical approximation of the calculated percentage-provide 2 subsequent semen samples with a clinically relevant decrease from a normal baseline in sperm concentration to <11 million sperm cells/mL (below the 2.5th percentile of WHO range of normality [WHO, 2021]), the dose will be escalated to the next planned dose level.

Part 1: If 100% of participants within a cohort experience a clinically relevant decrease from a normal baseline in sperm parameters on Day 28 that fall below the 2.5th percentile of WHO range of normality (WHO, 2010 and WHO, 2021), a maximally 3-fold higher dose level may be explored to further evaluate safety and tolerability.

Parts 2 and 3: If 100% of participants within a cohort experience a clinically relevant decrease from a normal baseline in sperm concentration by providing 2 subsequent semen samples with sperm concentrations <11 million sperm cells/mL (below the 2.5th percentile of WHO range of normality [WHO, 2021]), a maximally 3-fold higher dose level may be explored to further evaluate safety and tolerability.

See Section 8.5 for details on dose escalation stopping rules.

If, following review by the DERC it is deemed necessary and acceptable to continue dose escalation above the proposed maximum dose (180 mg), a substantial amendment with relevant supporting safety, PK and PD data will be submitted for approval to Medsafe or TGA.

Dosing decisions should be made in consultation with the Sponsor. The decision and all available safety, PK, and PD information will be documented.

Doses will be flexible during the study to allow for increases or reductions in the planned MAD escalation based on the safety and available PK and PD data from the preceding dosing period. When it is not appropriate to escalate the dose, then the same dose, a previously administered dose, or an intermediate dose may be given following DERC recommendations and further discussion with the Principal Investigator and Sponsor.

The dosing decisions and all information available will be documented and included in Clinical Study Report (CSR) Appendices after completion of the study.

### Citeria for Dose Selection for Cohort Expansion (Phase 2a [Part 3])

Dose levels will be selected based on emerging data from Part 2.

### Treatment Compliance

Participants will be counseled on the importance of adhering to study procedures. In Part 1, study drug will be administered at the designated clinical research unit on Days 1-7 under the supervision of trained site staff, who will ensure compliance with study drug administration procedures. On Days 8, 9, 11, 12, 13, 15, 16, 18, 19, 20, 22, 23, 25 and 26, participants will self-dose at home under supervision with a telehealth staff member via video call. On Days 10, 14, 17, 21, 24, 27 and 28, participants will self-dose at the designated clinical research unit under the supervision of trained site staff. Mouth and hand checks will be conducted after dosing to ensure the capsules have been swallowed. In Parts 2 and 3, participants will self-dose with study drug under the supervision of clinic staff on Days 1, 2, 14, 28, 42, 56, 70, 84 and 90. On those days, mouth and hand checks will be conducted after dosing to ensure the capsules have been swallowed. Further, participants will bring back the bottles with study drug from the previous dosing period for capsule accountability assessment. On Days 3-13, 15-27, 29-41, 43-55, 57-69, 71-83 and 85-89, participants will self-dose with no supervision. Participants are required to complete an electronic dosing diary daily on Days 1-90 after self-dosing.

### Method of Assigning Participants to Treatment Groups

In Part 1, 4 dosing cohorts and one optional 5th cohort with 4 participants will be evaluated (up to 20 participants). In Part 2, up to 4 dosing cohorts with 4 participants will be evaluated (up to 16 participants). All participants will receive Compound A. Participants may be replaced automatically prior to any dosing. If a participant is required to be replaced post-dose, the replacement may be approved at discretion of Sponsor and PI to ensure available data in at least 3 evaluable participants per cohort. In Part 3, up to 3 cohorts with up to 10 participants (up to 30 participants) will receive doses within the range of doses that was well tolerated and had biological activity in Part 2. All participants will receive Compound A. Participants who discontinue early may be replaced.

### Permitted and Prohibited Therapies

Concomitant medication use will be monitored by the Principal Investigator or designee during the sample collection period within the study and during any necessary washout periods.

All concomitant medications used (including over-the-counter medications and herbal supplements) will be recorded in the source document and on the appropriate eCRF.

### Permitted Therapies

All participants (except those who practice total abstinence) will be advised to use a condom during intercourse from the first administration of IMP until Day 56 (Part 1) or Day 118 (Parts 2 and 3).

Permitted therapies include up to 4 g of paracetamol or up to 3.2 g of ibuprofen per day and medications deemed necessary by the investigator to treat AEs. Other accepted concomitant medications may be accepted at the discretion of both the PI and the Sponsor.

COVID-19 and flu vaccines are accepted concomitant medications. It is preferable for participants not to receive the vaccine within 72 h prior to administration of IP, where possible, so that by the time of dosing any effects of the vaccine (e.g., pyrexia, fatigue, pain/stiffness at site of injection) are likely to have abated; however, participants would not be excluded on this ba-sis.

### Prohibited Therapies

No prescribed, over-the-counter medication or vitamins/herbal remedies supplements will be permitted from 14 days before IP administration until 28 Days after the last administration of study drug on Day 28 (Part 1) or Day 90 (Parts 2 and 3).

Participants may be excluded from the study if they are taking a medication that may, in the opinion of the investigator, affect the participant's well-being, the study conduct, or the interpretability of the results.

### Dispensing and Storage

The test product supplied by Quotient Sciences, the Sponsor's designated manufacturer, is to be used exclusively in the clinical study according to the instructions of this protocol. The pharmacy personnel are responsible for dispensing the study drug according to the dosage scheme and for ensuring that the study drug will be stored properly in accordance with the investigator's SOPs. Pharmacy personnel are required to wear personal protective equipment (PPE) when handling open bottles or capsules.

The pharmacy personnel must confirm the receipt of the study drug with his or her signature. A copy of this receipt must be kept by the pharmacy personnel and another copy will be stored by the Sponsor or an appropriate designee. Until the study drug is dispensed to the participants, it must be refrigerated (2 to 8° C.; 36 to 46° F.) and protected from light exposure in a securely locked area that is not generally accessible. Once dispensed to the participants, study drug can be stored at controlled room temperature (15 to 25° C.; 59 to 77° F.) protected from light exposure.

All study treatments will be stored in a secure, environmentally-controlled, and monitored (manual or automated) area in accordance with the labeled storage conditions with access limited to the investigator and authorized site staff.

### Drug Accountability

The pharmacy personnel must maintain adequate records showing the receipt, dispensing, return, or other disposition of the study drug, including the date, quantity, batch number, and identification of participants (participant number) who received the study drug. The pharmacy personnel will not supply the study drug to any person except those named as sub-investigators, designated study personnel, and participants in this study. The pharmacy personnel will not dispense the study drug from any study sites other than those listed and study drug may not be relabeled or reassigned for use by other participants. If any of the study drug is not dispensed, or is lost, stolen, spilled, unusable, or received in a damaged container, it will be removed from dosing and isolated away from to be dosed products in line with investigator's SOPs. This information must be documented and reported to the Sponsor and appropriate regulatory agencies, as required.

### Labeling and Packaging

### Labeling

Each container will have a label affixed that meets the applicable regulatory requirements and may include the following: participant identifier, study drug name, dosage strength, lot number, re-test date, protocol number, specified number of capsules, caution statement, storage, and Sponsor identification.

Final labeling will comply with the regulatory requirements of the country in which the study is conducted.

### Packaging

The investigational product will be packaged in plastic bottles; a more detailed description of the packaging will be provided in the pharmacy manual.

### Study Procedures

Participants must provide written informed consent before any study-related procedures are initiated, including the cessation of prohibited concomitant therapy. Throughout the study, every reasonable effort should be made by study personnel to follow the timing of assessments and procedures in the Schedules of Assessments for each participant. If a participant misses a study visit for any reason, the visit should be rescheduled as soon as possible.

### Study Duration

The total study duration in Part 1 from screening until the last follow-up visit (between Days 196 and 280) is up to 24-44 weeks. The total study duration in Parts 2 and 3 from screening until the last follow-up visit (up to Day 360) is up to 56 weeks.

### Primary Endpoints

The primary endpoint is the safety profile including the incidence of AEs, safety laboratory tests (clinical chemistry and liver function tests), CBC with differential (absolute and percentage), and lipid panel, ECGs (12-lead and Holter monitoring [Holter only in Part 1]), vital signs, and other safety assessments following repeated daily oral dosing of Compound A for 28 days (Part 1) or 90 days (Parts 2 and 3).

### Clinical Laboratory Safety Assessments

Blood samples will be collected and processed as detailed in the CSPM. Scheduled blood samples will be taken following a 10 h fast.

The acceptable deviations from the nominal blood sampling time points for laboratory assessments in Part 1 are:

Post-dose blood samples will be taken ±1 h from the nominal blood sampling time.

For return visits on Days 10, 14, 17, 21, 24, 56, 84, 112, 140, 168 196, and—if necessary—224, 252 and 280, blood samples will be taken ±4 hours from the nominal return visit time point.

The acceptable deviations from the nominal blood sampling time points for laboratory assessments in Parts 2 and 3 are:

On Days 1, 2, 90 and 91 (when participants reside in the clinic), post-dose blood samples will be taken ±1 h from the nominal blood sampling time.

For return visits on Days 14, 28, 42, 56, 70, 84, 92 and 93, post-dose blood samples will be taken ±2 hours from the nominal return visit time point. For return visits on Days 120, 150, 180, 210, 240, 270, 300, 330 and/or 360, blood samples will be taken ±4 hours from the nominal return visit time point.

Urinalysis will be performed on-site using a dipstick at the time points detailed in the schedule of assessments. Urine samples will be collected and processed as detailed in the CSPM. If microscopy is required, a urine sample will be sent to the responsible laboratory.

The acceptable deviations from the nominal urine sampling time points for urinalysis in Part 1 are:

Post-dose urine samples will be taken ±2 h from the nominal urine sampling time.

For return visits on Days 10, 14, 17, 21, 24, 56, 84, 112, 140, 168 196, and—if necessary—224, 252 or 280, urine samples will be taken ±4 hours from the nominal return visit time point.

The acceptable deviations from the nominal urine sampling time points for urinalysis in Parts 2 and 3 are:

Post-dose urine samples will be taken ±2 h from the nominal urine sampling time.

For return visits on Days 14, 28, 42, 56, 70, 84, post-dose urine samples will be taken ±4 hours from the nominal return visit time point. For return visits on Days 120, 150, 180, 210, 240, 270, 300, 330 and/or 360, urine samples will be taken ±4 hours from the nominal return visit time point.

### Vital Signs

Vital signs, including heart rate, and BP will be assessed. Heart rate and BP will be measured after the participant has been in a supine position for at least 5 minutes. Body temperature will also be measured as per SoA. The normal ranges for vital signs and body temperature are:
Systolic BP: 90-140 mmHg (<45 years old) and 90-160 mmHg (≥45 years old)
Diastolic BP: 40-90 mmHg
Heart rate: 45-100 beats/min
Oral temperature: 35.5-37.5° C.

The acceptable deviations from the nominal measurement time points in Part 1 are:

The pre-dose vital signs measurements will be taken ≤2 h before dosing.

Post-dose vital signs measurements on Days 1-6, 27 and 28 will be taken ±30 min from the nominal post-dose time points.

Discharge vital signs measurements on Days 7 and 31 will be taken ±1 h from the nominal time point.

For the return visits on Days 10, 14, 17, 21 and 24, vital signs measurements will be taken ±4 hours from the nominal return visit time point.

For the return visits on Days 56, 84, 112, 140, 168, 196, and—if necessary—Days 224, 252 or 280, vital signs measurements will be taken ±2 days from the nominal follow-up visit time point.

The acceptable deviations from the nominal measurement time points in Parts 2 and 3 are:

The pre-dose vital signs measurements will be taken ≤2 h before dosing.

Post-dose vital signs measurements on Days 1 and 90 will be taken ±30 min from the nominal post-dose time points.

Discharge vital signs measurements on Days 2 and 91 will be taken ±1 h from the nominal time point.

For the return visits on Days 14, 28, 42, 56, 70, 84, 92 and 93, vital signs measurements will be taken ±4 hours from the nominal return visit time point.

For the return visits on Days 120, 150, 180, 210, 240, 270, 300, 330 and/or 360, vital signs measurements will be taken ±2 days from the nominal follow-up visit time point.

On Day 89 (day of readmission), vital signs measurements will be taken at admission.

### Twelve-Lead Electrocardiogram

A standard 12 lead ECG will then be performed after the participant has been in a supine position for at least 5 minutes. The normal ECG ranges are:
Sinus bradycardia: ≤40 bpm
Sinus tachycardia: ≥100 bpm
PR interval: ≥120 to ≤220 msec
QRSD: <120 msec
QT: <500 msec
QTc: ≤450 msec

The acceptable deviations from the nominal 12-lead ECG measurement time points in Part 1 are:

Post-dose ECG measurements on Days 3-6 will be taken ±30 min from the nominal post-dose time point.

Discharge ECG measurements on Days 7 and 31 will be taken ±1 h from the nominal time point.

For the return visits on Days 10, 14, 17, 21 and 24, ECG measurements will be taken ±4 hours from the nominal return visit time point.

For the return visits on Days 56, 84, 112, 140, 168, 196, and—if necessary—Days 224, 252 or 280, ECG measurements will be taken ±2 days from the nominal follow-up visit time point.

The acceptable deviations from the nominal 12-lead ECG measurement time points in Parts 2 and 3 are:

Post-dose ECG measurements on Day 14 will be taken 6 hours ±30 min from the nominal post-dose time point.

Post-dose ECG measurements for the return visits on Days 28, 42, 56, 70 and 84, ECG measurements will be taken ±30 min from the nominal post-dose time point

For the return visits on Days 120, 150, 180, 210, 240, 270, 300, 330 and/or 360, ECG measurements will be taken ±2 days from the nominal follow-up visit time point.

### Holter (Continuous ECG) Monitoring

Holter monitoring will only be done in Part 1. Seventy-two (72)-hour continuous Holter monitoring is planned from Day −1 to Day 3. Forty-eight (48)-hour continuous Holter monitoring is planned from Day 28 to Day 30 to evaluate ECG readings for up to 72 hours after dosing. All ECGs will be collected electronically, and specific procedures for ECG Holter recording and extractions will be provided to the Investigator by the responsible laboratory.

Participants will be required to rest in the supine position with no external stimuli (e.g., music or television) for at least 10 min prior to the nominal time point where an ECG extraction is scheduled, and no other procedures should be performed within the resting period.

ECGs from the 72-hour Holter monitoring starting on Day −1 will be extracted at −24, −18, −12, −8, −6, −4, −2, −1, −0.5 h and −15 min prior to dosing on Day 1, and will commence to 0.5, 1, 2, 3, 4, 6, 8, 12, 18, 24, 24.5, 25, 26, 28, 30, 32, 36, 42 and 48 h post-Day 1 dosing.

ECGs from the 48-hour Holter monitoring starting on Day 28 will be extracted at 0.5, 1, 2, 4, 6, 8, 12, 18, 24 (Day 29), 25, 26, 28, 30, 32, 36, 42 and 48 h post-Day 28 dosing.

Where one period of ECG rest runs into the rest period for the next ECG extraction, the minimum resting period before ECG extraction does not need to be started again except in cases where the rest is disturbed (e.g., participant has to get up for a comfort break after the preceding rest period).

Participants may move more freely outside of the primary ECG rest periods. Comfort breaks for hygienic purposes will be allowed each morning well in advance of the required supine/ECG extraction points.

Where any scheduled rest times are missed, where there is less than a 10 min rest period or where the final 5 min of a rest period is interrupted (for example, by participant movement or loss of leads) a protocol deviation will be recorded and the Holter provider informed.

Loss of leads or interruption of the recording outside of the scheduled rest times will not be considered a protocol deviation but should be recorded in the source.

Post-dose ECG resting periods will be ended within +15 min of the nominal time point.

The ECGs will be collected electronically and over-read by cardiologists at the responsible Central ECG Laboratory. The responsible Central ECG Laboratory over-read will be used for data analysis and report writing purposes.

### Physical Examination

Complete physical examinations including neurological examination will be performed. In the targeted (symptom driven) physical examination, a physician will assess the participant within a reasonable time frame after a participant is reporting symptom(s); if the participant reports feeling unwell or has ongoing AEs, then the physician will examine the appropriate body system(s) if required. In addition, at all scheduled visits, symptoms and signs of depression, suicidal thoughts, and ideation as well as any self-harm behaviors will be evaluated as per the Investigator's judgement.

### Height and Weight

The participant's body weight and height will be measured, and their BMI will be calculated at the time points detailed in the schedule of assessments.

### Drug Screen

A urine drug screen will be performed on-site using a point of care testing method (e.g., Alere Drug Screen Test Cup) at the time points detailed in the schedule of assessments. The sample will be collected and processed as detailed in the CSPM.

### Alcohol Breath Test

An alcohol breath test will be performed on Day −1 as per Schedules of Assessments. A confirmed positive result will exclude the participant from dosing during that admission.

### Secondary and Exploratory Endpoints

The secondary endpoints are as follows:

Primary PD parameters: semen parameters including ejaculate volume, ejaculate pH, total sperm count per ejaculate, sperm concentration, and motility (total, progressive, non-progressive, immotile).

Plasma PK parameters: area under the curve from time 0 extrapolated to infinity (AUCinf), area under the curve from time 0 to the last measured concentration (AUC0-t), area under the curve from time 0 to 24 hours (AUC0-24), time to Cmax (Tmax), T1/2, lag time (Tlag), apparent volume of distribution (Vz/F), oral clearance [CL/F], and Cmax.

### Compound a Concentrations in Semen

Secondary PD parameters of Compound A, including serum hormones (FSH, LH, E2, T and calculated free T), and SHBG.

The exploratory endpoints are as follows:

Formal statistical analysis of relationship between the change from baseline in QTc interval using Bazett's and Fridericia's formulation (AQTcF) and plasma Compound A concentra-tion.

Formal statistical analysis of AQTcF at each post-dose timepoint for the comparison between each active dose level.

Exploratory PD parameters: levels of inflammatory biomarkers, including tumor necrosis fac-tor (TNF)-α, interleukin (IL)-6, high-sensitivity C-reactive protein (hsCR), troponin, von Willebrand factor (vWF) and E-selectin.

### Pharmacokinetic Assessments

Venous blood samples will be withdrawn via an indwelling cannula or by venipuncture at the time points detailed in the schedule of assessments.

The acceptable deviations from the nominal blood sampling times are as follows:

The pre-dose samples will be taken at −0.5 h before dosing.

0 to 1 h post-dose samples will be taken within +2 min of the nominal post-dose sampling time.

2 to 12 h post-dose samples will be taken within +10 min of the nominal post-dose sampling time.

18 h to 48 h post-dose samples will be taken within +30 min of the nominal post-dose sam-pling time.

>48 h post-dose samples will be taken within +4 h of the nominal post-dose sampling time.

In Part 1, semen PK samples will be collected once during the screening period (at least 5 days prior to dosing), on Days 21, 28, 56, and—if Compound A is present in semen on Day 56—also on Day 84. Sample collection should occur between 2 days and 5 days after the last ejaculation. In Parts 2 and 3, semen PK samples will be collected once during the screening period and on Days 84, 120 and 150.

Samples will be collected as described in the clinical sample processing manual.

### Pharmacodynamic Assessments

In Part 1, semen PD samples will be collected 3 times during the screening period, on Day 21 and Day 28 (±1 Day) during the dosing period, and on Days 42, 56, 84, 112, 140, 168 and 196. If sperm parameters have not returned to at least the 5th percentile of the WHO range of normality (WHO, 2010 and WHO, 2021; ≥15 million sperm cells/mL, ≥39 million/sperm cells in total ejaculate, ≥40% total motility, >30% progressive motility) by Day 196 (possible End of Study visit), semen samples will also be collected until the resolution of any clinically relevant changes in their sperm parameters (as assessed at visits on Days 224, 252, and 280 [all±2 days]). The last possible End of Study visit will be Day 280±2 days. In Parts 2 and 3, semen PD samples will be collected 3 times during the screening period and on Days 28, 42, 56, 70 and 84 (±2 Days) during the dosing period. Participants will be followed up for either 270 days after administration of the last dose (~4 spermatogenic cycles) or until two consecutive semen samples have sperm concentrations at baseline levels, whichever is earlier. In-person follow-up visits are planned on Day 98, 120, 150, 180, 210, 240, 270, 300, 330 and/or 360 (±2 days).

In Part 1, participants will have serum hormones (T, FSH, LH, E2 and calculated free T), and SHBG samples drawn once during the screening period, at −0.5 hours pre-dose (Day 1), and on Days 3, 7, 14, 21, 28, 56 (±2 Days), 112 (±2 Days; earliest End of Study Visit) and 196 (±2 Days; possible End of Study Visit); if necessary also on Days 224 or 252 (±2 Days; possible End of Study visits) or on Day 280 (±2 Days latest End of Study visit). In Parts 2 and 3, participants will have serum hormones (T, FSH, LH, E2 and calculated free T) and SHBG samples drawn once during the screening period, at −0.5 hours pre-dose (Day 1), and on Days 14, 28, 42, 56, 70, 84, 90, 120, 150, 180, 210, 240, 270, 300, 330 and/or 360 (±2 Days).

In Part 1, blood samples for evaluating general inflammatory biomarkers (TNF-α and IL-6) and biomarkers of early vascular endothelial injury (E-selectin, and vWF) will be collected once during the screening period, at −0.5 hours pre-dose (Day 1), and on Days 3, 7, 14, 21, 28, 56 (±2 Days), 112 (±2 Days; earliest End of Study Visit) and 196 (±2 Days; possible End of Study Visit); if necessary, also on Days 224 or 252 (±2 Days; possible End of Study visits) or on Day 280 (±2 Days; latest End of Study visit). In Parts 2 and 3, blood samples for evaluating hsCRP, troponin, TNF-α, IL-6, E-selectin and vWF will be collected once during the screening period, at −0.5 hours pre-dose (Day 1), and on Days 14, 28, 56, 42, 70, 84, 90, 120, 150, 180, 210, 240, 270, 300, 330 and/or 360 (+2 Days).

The acceptable deviations from the nominal blood sampling times are as follows:

The pre-dose samples will be taken ≤1 h before dosing.

24 h post-dose sample will be taken within +30 min of the nominal post-dose sampling time.

For all return visits, the sample will be taken ±4 hours from the nominal return visit time point.

### Other Study Assessments

### Self-Reported Psychosexual Diary

A self-reported diary (Appendix C) has been developed and used in other clinical studies to evaluate psychosexual function in men (Lee et al., 2003). In this study, it will only be used in Part 1. The diary scores parameters such as sexual desire, sexual enjoyment, sexual performance, sexual activity, and positive and negative moods. Participants are required to complete the diary at the first Screening visit, daily from Day −1 until Day 31 and at each follow-up visit post Day 31. The primary mode of completion of the diary will be electronically; however, completing the paper version is also acceptable. The completion of diary will be reviewed at the Screening visit, daily from Days-1 to 31 and at each follow-up visit post Day 31. The PI or designee will review all answers to Point 3 questions (mood changes) once per week and follow up with participant(s) if required.

### Dosing Diary

An electronic dosing diary will only be used in Parts 2 and 3. It needs to be completed daily by all Part 2 and Part 3 participants from Day 1-90 after each dosing. The completion of the diary will be reviewed daily. The primary mode of completion of the diary will be electronically; however, completing the paper version is also acceptable.

### Adverse Events

Adverse events will be monitored to assess the safety and tolerability of Compound A. The incidence and nature of any AEs, dose-limiting AEs (see Section 8.5), SAEs, vital sign changes, and laboratory abnormalities will be assessed on an ongoing basis.

### Definition and Classification of Adverse Events

### Adverse Events

An AE is any untoward medical occurrence in a participant that occurs either before dosing (referred to as a pre-dose AE) or once an investigational product, including placebo, has been ad-ministered, including occurrences which are not necessarily caused by or related to that product.

### Treatment-Emergent Adverse Events

An AE is defined as treatment-emergent if the first onset or worsening of the event occurs after the first administration of study drug and before the final Follow-up Visit.

### Adverse Drug Reaction

An adverse drug reaction (ADR) is any AE where a causal relationship with the IMP is at least a reasonable possibility (possibly related or related).

### Unexpected Adverse Event/Adverse Drug Reaction

An unexpected AE or unexpected ADR is one for which the nature or severity of the event is not consistent with the applicable product information (e.g., an IB for an unapproved investigational product or a package insert/summary of product characteristics for an approved product). As this is a first repeat dose human study, all AEs and ADRs are considered unexpected.

### Serious Adverse Events

A SAE is any untoward medical occurrence that at any dose:

Results in death.

Is life-threatening.

NOTE: The term “life-threatening” in the definition of “serious” refers to an event in which the participant was at risk of death at the time of the event; it does not refer to an event that hypothetically might have caused death if it were more severe.

Requires in-patient hospitalization or prolongation of existing hospitalization.

NOTE: In-patient hospitalization is defined as 24 hours in a hospital or an overnight stay. An elective hospital admission to treat a condition present before exposure to the investigational product or a hospital admission for a diagnostic evaluation of an AE does not qualify the condition or event as an SAE. Further, an overnight stay in the hospital that is only due to transportation, organization, or accommodation problems and without medical background does not need to be considered an SAE.

Results in persistent or significant disability/incapacity.

Is a congenital anomaly.

NOTE: A congenital anomaly in an infant born to a mother who was exposed to the investigational product during pregnancy is an SAE. However, a newly diagnosed pregnancy in a participant who has received an investigational product is not considered an SAE unless it is suspected that the investigational product(s) interacted with a contraceptive method and led to the pregnancy.

Is an important medical event.

NOTE: Medical and scientific judgment should be exercised in deciding whether it is ap-propriate to consider other situations serious, such as important medical events that may not be immediately life-threatening or result in death or hospitalization but may jeopardize the participant or require intervention to prevent one of the other outcomes listed in the definition above. Examples of such events are intensive treatment in an emergency room or at home for allergic bronchospasm, blood dyscrasias, or convulsions that do not result in hospitalization, development of drug dependency, or drug abuse.

### Suspected Unexpected Serious Adverse Reactions

Suspected unexpected serious adverse reactions (SUSARs) are AEs that are believed to be related to an IMP and are both unexpected (i.e., the nature or severity is not expected from the information provided in the IB) and serious. SUSARs are participant to expedited reporting to Medsafe or TGA.

### Recording of Adverse Events

AEs (including SAEs) will be recorded from the time of providing written informed consent until discharge from the study at the follow-up phone call. During each study visit the participant will be questioned directly regarding the occurrence of any adverse medical event according to the schedule in the source. All AEs, whether ascribed to study procedures or not, will be documented immediately in the participant's source. This will include the date and time of on-set, a description of the AE, severity, seriousness, duration, actions taken, outcome and an investigator's current opinion on the relationship between the study drug and the event. A diagnosis and final opinion on the relationship between the study drug and the event will be provided at the end of the study by the investigator.

Any participant who withdraws from the study due to an AE will be followed up until the outcome is determined and written reports are provided by the investigator.

### Assessment of Severity

The clinical severity of an AE will be classified as described below.

Mild: An AE that is easily tolerated by the participant, causes minimal discomfort and does not interfere with everyday activities.

Moderate: An AE that is sufficiently discomforting to interfere with normal everyday activities; intervention may be needed.

Severe: An AE that prevents normal everyday activities; treatment or other intervention usually needed.

It is important to distinguish between severe AEs and SAEs. Severity is a classification of in-tensity whereas an SAE is an AE that meets serious criteria.

### Adverse Event Relationship to Investigational Product

The investigator must make an assessment of each AE's relationship to the study drug. The categories for classifying the investigator's opinion of the relationship are as follows:

Related: Clinical event with plausible time relationship to IMP administration and that cannot be explained by concurrent disease or other drugs or chemicals.

Possibly related: Clinical event with a reasonable time relationship to IP administration, and that is unlikely to be attributed to concurrent disease or other drugs or chemicals.

Not related: Clinical event with an incompatible time relationship to IP administration, and that could be explained by underlying disease or other drugs or chemicals or is incontrovertibly not related to the IP.

The relationship between study drug and the AE may be assessed separately by the investigator and the Sponsor.

The degree of certainty with which an AE is attributed to IP administration (or alternative causes, e.g., natural history of the underlying disease, concomitant therapy) will be determined by how well the experience can be understood in terms of one or more of the following:

Known pharmacology of the IP

Reactions of a similar nature have been previously observed with the IP or this class of drug.

The experience is related by time to IP administration.

Alternative cause

### Adverse Events of Special Interest

Adverse events of special interest (AESIs) were selected based on available nonclinical safety information. The following events will be considered AESIs:

Prolongation of QTc (>500 msec or >70 msec above baseline)

Protracted (>1 hour) nausea and vomiting without an alternative explanatory cause.

Any related AE deemed by the investigator to be dose-limiting (see Section 8.5) or Common Terminology Criteria for Adverse Events (CTCAE version 5.0) Grade 3 and above.

Clinical investigations including signs of DILI based on following criteria from FDA guidance:
ALT or AST >8× upper limit of normal (ULN)
ALT or AST >5×ULN for more than 2 weeks
ALT or AST >3×ULN and (total bilirubin >2×ULN or international normalized ratio >1.5)
ALT or AST >3×ULN with the appearance of fatigue, nausea, vomiting, right upper quadrant pain or tenderness, fever, rash, and/or eosinophilia (>5%)

Clinically significant changes in complete blood count, coagulation, and blood chemistry

Clinically significant changes from baseline in renal function

### Potential Clinical Significance

Selected clinical laboratory results that may be of potential clinical significance were selected based on available safety data from animal studies. Results of interest include values outside normal range that persist for the duration of the study and have matching increases in related parameters and clinical presentations. Laboratory results of potential clinical significance may be identified at the discretion of the Principal Investigator and DERC and may include the parameters listed below. If a cutoff for potential clinical significance is not explicitly listed for a given parameter in the list below, the cutoff value will be based on the threshold for Grade 2 events as defined by the CTCAE version 5.0.

### Serum Chemistry:

Increased: ALT, AST, alkaline phosphatase, gamma glutamyl transferase, lactate dehydrogenase, total bilirubin (>1.5 to 3.0×ULN if baseline was normal; >1.5 to 3.0× baseline if baseline was abnormal), creatinine (>1.5 to 3.0× baseline; >1.5 to 3.0×ULN)

Events of potential clinical significance will include ECG abnormalities (criteria listed above), liver injury (criteria listed above), and significant changes in blood values.

### Pregnancy

All participants who enroll in the study will be male.

Male participants with partner(s) of childbearing potential will be advised to use effective contraception including condom contraception from first administration of IMP until Day 56 (Part 1) or Day 118 (Parts 2 and 3). Total abstinence from intercourse during the course of the study until Day 56 is considered an acceptable form of contraception if this is in line with participant's preferred and/or usual lifestyle. Condom use is not required while practicing total abstinence.

Male participants with partner(s) of non-childbearing potential will also be required to use a condom from first administration of IMP until Day 56 (Part 1) or Day 118 (Parts 2 and 3) to avoid the potential risk of drug exposure through the ejaculate.

### Overdose

Overdose that occurs during the study will be treated and documented as an AE/SAE if it fulfills the appropriate criteria. If the overdose does not result in an AE, it should be reported in written form to the designated individual(s) who receive SAE notification. The information contained therein should include study site identification, reporter identification, participant identification, study drug, dose, action taken (e.g., supportive measures or therapy), and any comments.

### Statistics

### Statistical Analysis

This section presents a summary of the planned statistical analyses. A reporting and analysis plan (RAP) that describes the details of the analyses to be conducted will be written prior to database lock.

Summary statistics will generally be provided for study variables. For continuous variables, these statistics will typically include the number of participants, mean, standard deviation (SD), median, minimum, and maximum including changes from baseline as appropriate. For categorical variables, these statistics will typically include the number and percentage of participants in each category.

### Analysis Populations

The following analysis populations are planned for this study:

Screening Population: all participants who provide informed consent and demographic and/or baseline Screening assessments, regardless of the participant's randomization and treatment status in the study.

Safety Population: all participants who receive study drug.

Per Protocol Population: all participants who receive at least 80% of the planned doses of study drug.

Pharmacokinetic Population in Part 1: all participants who receive study drug and have at least 7 PK samples collected on Days 1 and 28.

Pharmacokinetic Population in Parts 2 and 3: all participants who receive study drug and have at least 7 PK samples collected on Days 1 and 90.

Pharmacodynamic Population in Part 1: all participants who receive study drug and have at least 2 evaluable semen samples collected starting on Day 21 or later during the study.

Pharmacodynamic Population in Parts 2 and 3: all participants who receive study drug and have at least 4 evaluable semen samples collected starting on Day 28 during the dosing phase, and at least 4 evaluable semen samples collected starting on Day 120 during the recovery phase.

The Sponsor will identify major protocol deviations prior to database lock.

Inclusion in the analysis populations will be determined prior to database lock.

If a participant is randomized incorrectly or is administered the incorrect study drug, analyses of the Per Protocol Population will be based on the assigned treatment whereas all other analyses will be based on the actual treatment.

This disclosure is not limited to the particular systems, devices and methods described, as these may vary. The terminology used in the description is for the purpose of describing the particular versions or embodiments only and is not intended to limit the scope.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present.

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 compounds refers to groups having 1, 2, or 3 compounds. Similarly, a group having 1-5 compounds refers to groups having 1, 2, 3, 4, or 5 compounds, and so forth.

Various of the above-disclosed and other features and functions, or alternatives thereof, may be combined into many other different systems or applications. Various presently unforeseen or unanticipated alternatives, modifications, variations, or improvements therein may be subsequently made by those skilled in the art, each of which is also intended to be encompassed by the disclosed embodiments.

Embodiments

1. A method of reducing the likelihood of conception following intercourse between a male subject and a female subject, comprising administering to the male subject prior to the intercourse a therapeutically effective amount of a compound of formula (I): 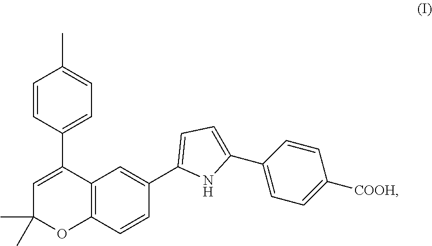 [SMILES: Cc1ccc(C2=CC(C)(C)Oc3ccc(-c4ccc(-c5ccc(C(=O)O)cc5)[nH]4)cc32)cc1]
or a pharmaceutically acceptable salt thereof;
wherein the therapeutically effective amount is about 15 mg to about 450 mg.

2. The method of claim 1, wherein the therapeutically effective amount is about 15 mg to about 180 mg.

3. The method of claim 1, wherein the therapeutically effective amount is about 15 mg to about 80 mg.

4-8. (canceled)

9. The method of claim 1, wherein the therapeutically effective amount is administered once per day for about 28 days to about 24 weeks.

10. The method of claim 1, wherein the therapeutically effective amount is administered once per day for about 13 weeks to about 24 weeks.

11-13. (canceled)

14. A method of producing reversible infertility in a male subject, comprising administering to the male subject comprising administering to the male subject a therapeutically effective amount of a compound of formula (I): 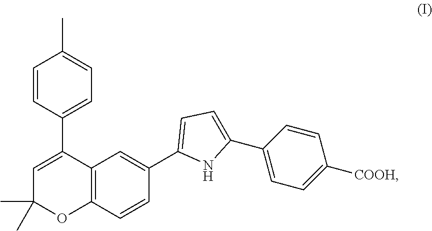 [SMILES: Cc1ccc(C2=CC(C)(C)Oc3ccc(-c4ccc(-c5ccc(C(=O)O)cc5)[nH]4)cc32)cc1]
or a pharmaceutically acceptable salt thereof;
wherein the therapeutically effective amount is about 15 mg to about 450 mg.

15. The method of claim 14, wherein the therapeutically effective amount is about 15 mg to about 180 mg.

16. The method of claim 14, wherein the therapeutically effective amount is about 15 mg to about 80 mg.

17-21. (canceled)

22. The method of claim 14, wherein the therapeutically effective amount is administered once per day for about 28 days to about 24 weeks.

23. The method of claim 14, wherein the therapeutically effective amount is administered once per day for about 13 weeks to about 24 weeks.

24-26. (canceled)

27. A method of reducing sperm count in a male subject, comprising administering to the male subject a therapeutically effective amount of a compound of formula (I): 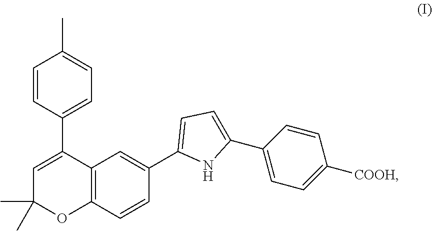 [SMILES: Cc1ccc(C2=CC(C)(C)Oc3ccc(-c4ccc(-c5ccc(C(=O)O)cc5)[nH]4)cc32)cc1]
or a pharmaceutically acceptable salt thereof;
wherein the therapeutically effective amount is about 15 mg to about 450 mg.

28. The method of claim 27, wherein the therapeutically effective amount is about 15 mg to about 180 mg.

29. The method of claim 27, wherein the therapeutically effective amount is about 15 mg to about 80 mg.

30-34. (canceled)

35. The method of claim 27, wherein the therapeutically effective amount is administered once per day for about 28 days to about 24 weeks.

36. The method of claim 27, wherein the therapeutically effective amount is administered once per day for about 13 weeks to about 24 weeks.

37-39. (canceled)

40. A method of selectively antagonizing RAR alpha over RAR beta and RAR gamma in a subject, comprising administering to the subject a therapeutically effective amount of a compound of formula (I): 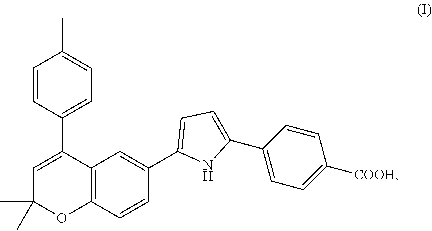 [SMILES: Cc1ccc(C2=CC(C)(C)Oc3ccc(-c4ccc(-c5ccc(C(=O)O)cc5)[nH]4)cc32)cc1]
or a pharmaceutically acceptable salt thereof;
wherein the therapeutically effective amount is about 15 mg to about 450 mg.

41. The method of claim 40, wherein the therapeutically effective amount is about 15 mg to about 180 mg.

42. The method of claim 40, wherein the therapeutically effective amount is about 15 mg to about 80 mg.

43-47. (canceled)

48. The method of claim 40, wherein the therapeutically effective amount is administered once per day for about 28 days to about 24 weeks.

49. The method of claim 40, wherein the therapeutically effective amount is administered once per day for about 13 weeks to about 24 weeks.

50-52. (canceled)
